(19) United States
(12) Patent Application Publication
(10) Pub. No.: US 2023/0128917 A1
(43) Pub. Date: Apr. 27, 2023

(54) GENETICALLY ENGINEERED IMMUNE CELLS HAVING A DISRUPTED CD83 GENE

(71) Applicant: CRISPR Therapeutics AG, Zug (CH)

(72) Inventors: Daniel Robert Hostetter, Cambridge, MA (US); Ismael Oumzil, Cambridge, MA (US); Brian Christopher Fochtman, Cambridge, MA (US); John Michael Louis Toomey, Cambridge, MA (US)

(21) Appl. No.: 17/931,701

(22) Filed: Sep. 13, 2022

Related U.S. Application Data
(60) Provisional application No. 63/244,050, filed on Sep. 14, 2021.
(60) Provisional application No. 63/254,332, filed on Oct. 11, 2021.
(60) Provisional application No. 63/303,666, filed on Jan. 27, 2022.

(51) Int. Cl. A61K 35/17; C07K 14/705; C07K 14/725

(52) U.S. Cl. CPC A61K 35/17; C07K 14/70521; C07K 14/7051; A61K 2039/5156

(57) ABSTRACT

Genetically engineered immune cells such as T cells having a disrupted CD83 gene and optionally expressing a chimeric antigen receptor (CAR) and therapeutic applications thereof. Such genetically engineered immune cells may further comprise a disrupted TRAC gene, a disrupted β2M gene, or a combination thereof.

### CROSS REFERENCE TO RELATED APPLICATIONS

This application claims the benefit of U.S. Provisional Application No. 63/244,050, filed Sep. 14, 2021, U.S. Provisional Application No. 63/254,332, filed Oct. 11, 2021, and U.S. Provisional Application No. 63/303,666, filed Jan. 27, 2022. The entire contents of each of the prior applications are incorporated by reference herein.

### SEQUENCE LISTING

The instant application contains a Sequence Listing which has been filed electronically in XML format and is herein incorporated by reference in its entirety. Said XML copy, created on Sep. 13, 2022, is named 095136-0713-054US1_SEQ.xml and is 264,232 bytes in size.

### BACKGROUND OF THE INVENTION

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

T cells having improved expansion capacity, in vitro and/or in vivo, are desired in CAR T cell manufacturing and clinical applications.

### SUMMARY OF THE INVENTION

The present disclosure is based, at least in part, on the development of genetically engineered immune cells such as T cells (e.g., anti-CD83 CAR-T cells) having a disrupted CD83 gene. Relative to T cell counterparts having a wild-type CD83 gene, such genetically engineered immune cells showed enhanced overall T cell expansion and/or reduced levels of pro-inflammatory cytokine production with no negative impact on CAR-T cell function. The genetically engineered immune cells are also expected to have reduced graft versus host (GvHD) responses when administered to a subject.

Accordingly, some aspects of the present disclosure feature a population of genetically engineered immune cells, comprising a disrupted CD83 gene. In some instances, the genetically engineered immune cells comprise T cells. In some instances, the immune cells may be further engineered to express a chimeric antigen receptor (CAR). The population of genetically engineered immune cells as disclosed herein may be derived from one or more human donors.

In some embodiments, the disrupted CD83 gene may be genetically edited in exon 1, exon 2, exon 3, or exon 4. In some examples, the disrupted CD83 gene may be genetically edited in exon 2. In some examples, the disrupted CD83 gene may be genetically edited by a CRISPR/Cas-mediated gene editing system. For example, the CRISPR/Cas-mediated gene editing system may comprise a guide RNA (gRNA) targeting a site in the CD83 gene. The site in the CD83 gene may comprises a nucleotide sequence of SEQ ID NO: 22, 28, 72, 74, 76, 78, 80, 82, 84, 86, 88, 90, 92, 94, 96, 98, 161, 167, 169, 171, 173, 187, 189, 191, 193, 195, or 197. In some instances, the site in the CD83 gene may comprise the nucleotide sequence of SEQ ID NO: 28. In some instances, the site in the CD83 gene may comprise the nucleotide sequence of SEQ ID NO: 74. In some instances, the site in the CD83 gene may comprise the nucleotide sequence of SEQ ID NO: 88. In some instances, the site in the CD83 gene may comprise the nucleotide sequence of SEQ ID NO: 169.

In some examples, the gRNA targeting the CD83 gene may comprise a spacer having a nucleotide sequence of SEQ ID NOs: 19, 25, 101, 105, 109, 113, 117, 121, 125, 129, 133, 137, 141, 145, 149, 153, 158, 164, 176, 180, 184, 200, 204, 208, 212, 216, or 220. In some instances, the gRNA targeting the CD83 gene may comprise a spacer having the nucleotide sequence of SEQ ID NO: 25. In some instances, the gRNA targeting the CD83 gene may comprise a spacer having the nucleotide sequence of SEQ ID NO: 105. In some instances, the gRNA targeting the CD83 gene may comprise a spacer having the nucleotide sequence of SEQ ID NO: 133. In some instances, the gRNA targeting the CD83 gene may comprise a spacer having the nucleotide sequence of SEQ ID NO: 176.

Any of the gRNAs disclosed herein may further comprise a scaffold sequence (e.g., SEQ ID NO: 155 or 234). In specific examples, the gRNA targeting the CD83 gene may comprise a nucleotide sequence of SEQ ID NOs: 17, 18, 23, 24, 99, 100, 103, 104, 107, 108, 111, 112, 115, 116, 119, 120, 123, 124, 127, 128, 131, 132, 135, 136, 139, 140, 143, 144, 147, 148, 151, 152, 156, 157, 162, 163, 174, 175, 178, 179, 182, 183, 198, 199, 202, 203, 206, 207, 210, 211, 214, 215, 218, or 219. See Table 2 below, all of the gRNAs targeting the CD83 gene listed in which are within the scope of the present disclosure.

In some instances, the gRNA targeting the CD83 gene may comprise the nucleotide sequence of SEQ ID NO: 23 or SEQ ID NO: 24. In some instances, the gRNA targeting the CD83 gene may comprise the nucleotide sequence of SEQ ID NO: 103 or SEQ ID NO: 104. In some instances, the gRNA targeting the CD83 gene may comprise the nucleotide sequence of SEQ ID NO: 131 or SEQ ID NO: 132. In some instances, the gRNA targeting the CD83 gene may comprise the nucleotide sequence of SEQ ID NO: 174 or SEQ ID NO: 175.

In some embodiments, any of the genetically engineered immune cells disclosed herein, which may comprise T cells, may further comprise a disrupted T cell receptor alpha chain constant region (TRAC) gene, a disrupted beta-2-microglobulin (32M) gene, or a combination thereof. In some examples, the disrupted TRAC gene is genetically edited by a CRISPR/Cas-mediated gene editing system, which comprises a gRNA comprising the nucleotide sequence of SEQ ID NO: 4. See Table 1 below. In some instances, the disrupted TRAC gene may comprise a nucleotide sequence of any one of SEQ ID NOs: 29-36. See Table 3 below.

In some examples, the T cells further comprise the disrupted β2M gene. For example, the disrupted β2M gene is genetically edited by a CRISPR/Cas-mediated gene editing system, which comprises a gRNA comprising the nucleotide sequence of SEQ ID NO: 8. Alternatively, the disrupted β2M gene is genetically edited by a CRISPR/Cas-mediated gene editing system, which comprises a gRNA comprising the nucleotide sequence of SEQ ID NO: 224. See Table 1 below. In some instances, the disrupted β2M gene may comprise a nucleotide sequence of any one of SEQ ID NOs: 37 to 42. See Table 4 below.

In some examples, the population of genetically engineered immune cells disclosed herein may comprise T cells, which comprise a nucleic acid encoding the CAR. The nucleic acid encoding the CAR may be inserted in a genomic site of interest in the T cells. In some instances, the genomic site of interest is the TRAC gene. In one example, the disrupted TRAC gene comprises the nucleic acid encoding the CAR.

Any of the CAR disclosed herein may comprise an extracellular antigen binding domain specific to an antigen, a co-stimulatory signaling domain of 4-1BB or CD28, and a cytoplasmic signaling domain of CD3ζ. In some embodiments, the CAR disclosed herein comprise an extracellular antigen binding domain capable of binding to CD83.

In some examples, the extracellular antigen binding domain in a CAR disclosed herein is a single chain variable fragment (scFv) that binds CD83 (anti-CD83 scFv). The anti-CD83 scFv may comprise a heavy chain variable region (V_{H}) and a light chain variable region (V_{L}). In some instances, the V_{H} and V_{L} can be connected via a peptide linker. In some examples, the V_{H} may comprise heavy chain complementary determining region (CDR) 1, CDR2, and CDR3 set forth as SEQ ID NOs: 43, 44, and 45, respectively. Alternatively or in addition, the V_{L} may comprise light chain complementary determining region (CDR) 1, CDR2, and CDR3 set forth as SEQ ID NOs: 46, 47, and 48, respectively. In specific examples, the V_{H} may comprise the amino acid sequence of SEQ ID NO: 49, and/or the V_{L} comprises the amino acid sequence of SEQ ID NO: 50. In one example, the anti-CD83 scFv comprises the amino acid sequence of SEQ ID NO: 51. In another example, the CAR that binds CD83 comprises the amino acid sequence of SEQ ID NO: 52, or the mature form thereof without the N-terminus signal peptide (italicized) (SEQ ID NO: 235). See Table 5 below.

In other aspects, the present disclosure provides a method for preparing the population of genetically engineered immune cells of claim 1, the method comprising:
(a) providing a plurality of immune cells, which optionally comprise T cells or precursor cells thereof;
(b) genetically editing a CD83 gene of the immune cells; and
(c) producing the population of genetically engineered immune cells having a disrupted CD83 gene.

In some embodiments, step (b) can be performed by delivering to the plurality of immune cells an RNA-guided nuclease and a gRNA targeting the CD83 gene. The gRNA may be specific to any one of exon 1 to exon 4 of the CD83 gene. Exemplary gRNAs targeting CD83 are provided in Table 2 below. In some examples, the gRNA may target exon 2 of a CD83 gene. In some instances, the gRNA targeting the CD83 gene may comprise a spacer having a nucleotide sequence of any one of SEQ ID NOs: 25, 105, 133, and 176. The gRNA further comprises a scaffold sequence. In some examples, the gRNA may comprise a nucleotide sequence of any one of SEQ ID NOs: 23, 24, 103, 104, 131, 132, 174 and 175.

In some embodiments, the plurality of immune cells in step (a) comprises one or more of the following genetic modifications: (i) engineered to express a chimeric antigen receptor (CAR); (ii) has a disrupted T cell receptor alpha chain constant region (TRAC) gene; and (iii) has a disrupted β2M gene. In some embodiments, the immune cells of step (a) are derived from one or more human donors.

In other embodiments, the preparation method disclosed herein may comprise:
(i) delivering to the T cells a nucleic acid encoding a chimeric antigen receptor (CAR);
(ii) genetically editing a TRAC gene to disrupt its expression;
(iii) genetically editing a β2M gene to disrupt its expression; or
(iv) a combination thereof.

In some examples, (ii) and/or (iii) may be performed by one or more CRISPR/Cas-mediated gene editing systems comprising one or more RNA-guided nucleases and one or more gRNAs targeting the TRAC gene, the β2M gene, or a combination thereof. In some instances, the gRNA targeting the TRAC gene may comprise the nucleotide sequence of SEQ ID NO: 4.

Alternatively or in addition, the gRNA targeting the β2M gene may comprise the nucleotide sequence of SEQ ID NO: 8 or SEQ ID NO: 224.

In some embodiments, the method disclosed herein may comprise delivering to the immune cells one or more ribonucleoprotein particles (RNP), which comprises the RNA-guided nuclease, and one or more of the gRNAs. In some examples, the RNA-guided nuclease may be a Cas9 nuclease, for example, a S. pyogenes Cas9 nuclease.

In some embodiments, the nucleic acid encoding the CAR is in an AAV vector. In some examples, the nucleic acid encoding the CAR may comprise a left homology arm and a right homology arm flanking the nucleotide sequence encoding the CAR. The left homology arm and the right homology arm are homologous to a genomic locus in the T cells, allowing for insertion of the nucleic acid into the genomic locus. In some examples, the genomic locus is in the TRAC gene. In some examples, the method may comprise disrupting the TRAC gene by a CRISPR/Cas-mediated gene editing system comprising the gRNA that comprises the nucleotide sequence of SEQ ID NO: 4 and the nucleic acid encoding the CAR is inserted at the site targeted by the gRNA.

Also within the scope of the present disclosure is a population of genetically engineered T cells, which is prepared by any of the preparation methods disclosed herein. Further, provided herein are any of the genetically engineered immune cells such as T cells as disclosed herein for use in eliminating undesired disease cells or treating a target disorder as disclosed herein (e.g., cancer or an immune disorder such as an autoimmune disease). Also provided herein are use of any of the genetically engineered immune cells such as T cells for manufacturing a medicament for use in treating any of the target diseases.

Further, the present disclosure provides a method for eliminating undesired cells in a subject, the method comprising administering to a subject in need thereof a first population of genetically engineered immune cells comprising a disrupted CD83 gene and expressing a first chimeric antigen receptor (CAR) targeting the undesired cells. The first population of genetically engineered immune cells may be any of such disclosed in the present disclosure. In some examples, the undesired cells are CD83+ cells and the first CAR binds CD83.

Any of the methods may further comprise administering to the subject a second population of genetically engineered immune cells expressing a second chimeric antigen receptor (CAR) specific to a tumor antigen, for example, CD19, BCMA, or CD70. In some instances, the first and second populations of genetically engineered immune cells overlap, which comprise genetically engineered immune cells expressing both the first CAR and the second CAR. In some instances, the subject may be a human patient suffering from a cancer.

Moreover, the present disclosure provides a guide RNA (gRNA) targeting a CD83 gene, comprising a nucleotide sequence specific to a fragment in exon 2 or exon 3 of the CD83 gene. In some embodiments, the gRNA targeting CD83 may comprise a spacer having the nucleotide sequence of SEQ ID NO: 19, 25, 101, 105, 109, 113, 117, 121, 125, 129, 133, 137, 141, 145, 149, 153, 158, 164, 176, 180, 184, 200, 204, 208, 212, 216, or 220. See Table 2. Any of the guide RNAs disclosed herein may further comprise a scaffold sequence (e.g., SEQ ID NO: 155 or 234). In some instances, the gRNAs disclosed herein may comprise one or more modified nucleotides. For example, the gRNA may comprise one or more 2′-O-methyl phosphorothioate residues at the 5′ and/or 3′ terminus of the gRNA. In specific examples, the gRNA targeting CD83 may comprise the nucleotide sequence of any one of SEQ ID NO: SEQ ID NOs: 17, 18, 23, 24, 99, 100, 103, 104, 107, 108, 111, 112, 115, 116, 119, 120, 123, 124, 127, 128, 131, 132, 135, 136, 139, 140, 143, 144, 147, 148, 151, 152, 156, 157, 162, 163, 174, 175, 178, 179, 182, 183, 198, 199, 202, 203, 206, 207, 210, 211, 214, 215, 218, or 219. See Table 2 below, all of the gRNAs targeting the CD83 gene listed in which are within the scope of the present disclosure.

In some instances, the gRNA targeting the CD83 gene may comprise the nucleotide sequence of SEQ ID NO: 23 or SEQ ID NO: 24. In some instances, the gRNA targeting the CD83 gene may comprise the nucleotide sequence of SEQ ID NO: 103 or SEQ ID NO: 104. In some instances, the gRNA targeting the CD83 gene may comprise the nucleotide sequence of SEQ ID NO: 131 or SEQ ID NO: 132. In some instances, the gRNA targeting the CD83 gene may comprise the nucleotide sequence of SEQ ID NO: 174 or SEQ ID NO: 175.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic illustration showing location of exemplary anti-CD83 guide RNAs relative to exons of the CD83 gene and functional domains within CD83.

FIGS. 2A-2C include diagrams showing impact of CD83 disruption on production of pro-inflammatory cytokines in anti-CD83 CAR-T cells. 2A: IL-2. 2B: TNFα. 2C: IFNγ.

FIG. 3 is a diagram showing cytotoxicity activity of CAR-T cells with and without CD83 disruption.

FIGS. 4A-4C include diagrams showing impact of CD83 disruption on cell growth and frequency of CD8/CD4 subtypes. FIGS. 4A-4B: growth curves across different cultures. FIG. 4C: viability across different cultures.

FIGS. 5A and 5B include diagrams showing cytotoxicity activity of anti-CD83 CAR T cells with CD83 disruption by different guides. FIG. 5A: cytotoxicity activity at E:T ratio ranging from 8:1 to 0.0625:1. FIG. 5B: cytotoxicity activity at E:T ratio ranging from 0.5:1 to 0.0625:1.

FIG. 6 is a diagram shown that anti-CD83 CAR T cells with disruption of the CD83 gene improved animal survival rates in a mouse graft-versus-host disease (GvHD) model, as compared with the counterpart anti-CD83 CAR-T cells with no CD83 gene disruption.

FIG. 7 is a diagram shown that anti-CD83 CAR T cells with CD83 gene disruption enhanced survival rates in a mouse THP1 xenograft tumor model, as compared with the counterpart anti-CD83 CAR-T cells with no CD83 gene disruption.

### DETAILED DESCRIPTION OF THE INVENTION

The present disclosure aims at establishing genetically engineered T cells having improved growth activity, reduced production of pro-inflammatory cytokines, increased CD8⁺ cell frequency, and/or enhanced potency of CAR-T cells such as anti-CD83 CAR-T cells. Such genetically engineered T cells may use bonafide T cells as the starting material, for example, non-transformed T cells, terminally differentiated T cells, T cells having stable genome, and/or T cells that depend on cytokines and growth factors for proliferation and expansion. Alternatively, such a T cell may use T cells generated from precursor cells such as hematopoietic stem cells (e.g., iPSCs), e.g., in vitro culture. The T cells disclosed herein may confer one or more benefits in both CAR-T cell manufacturing and clinical applications.

Conventional allogenic CAR T cells are produced using genetically edited T cells from a single donor leukopak so that the cells can avoid components of the patient immune system and thus do not cause GvHD. The process of expanding these CAR T cells can yield 10s to 100s of vialed drug product. Patients may receive a single dose or multiple doses. During the manufacturing process, these CAR T cells lose potential due to various mechanisms, for example, apoptosis, exhaustion, replicative senescence, and other processes where the cells become less fit.

The genetically engineered T cells having a disrupted CD83 gene and optionally one or more additional genetic edits, for example, a disrupted TRAC gene and/or a disrupted 82M gene. In addition, the genetically engineered T cells may be engineered to express a chimeric antigen receptor (CAR) (e.g., a CAR capable of binding to CD83 or an anti-CD83 CAR). Such genetically engineered T cells may comprise a nucleic acid encoding the CAR. In some instances, the nucleic acid encoding the CAR may be inserted at a genomic site of interest, for example, in the disrupted TRAC gene.

Unexpectedly, the present disclosure reports that disrupting the CD83 gene led to reduced production of pro-inflammatory cytokine production, enhanced T cell expansion, and increased CD8+ cell frequency with no negative impact on CAR-T cell function, as compared with CAR-T cell counterparts carrying a wild-type CD83. Further, CD83 disruption prevents cells from prematurely reaching T cell exhaustion and increases potency and persistence in vivo. CAR-T cell counterparts refer to genetically engineered T cells having the same genetic edits except for status of the CD83 gene.

Accordingly, provided herein are a population of genetically engineered immune cells (e.g., CAR-T cells) comprising a disrupted CD83 gene, methods for preparing such genetically engineered immune cells, and methods of using such genetically engineered immune cells for eliminating undesired cells (e.g., cancer cells) in a subject in need of the treatment. Also provided herein are components (e.g., guide RNAs) and systems (e.g., a CRISPR/Cas9 gene editing system) for disrupting the CD83 gene in T cells.

### I. Genetically Engineered Immune Cells Having Enhanced Features

The T cells disclosed herein comprises genetically engineered immune cells such as T cells or NK cells having genetic editing of the CD3 gene.

The genetically engineered immune cells (e.g., T cells) may be derived from parent immune cells (e.g., non-edited wild-type immune cells such as T cells) obtained from a suitable source, for example, one or more mammal donors. In some examples, the parent T cells are primary T cells (e.g., non-transformed and terminally differentiated T cells) obtained from one or more human donors. Alternatively, the parent T cells may be differentiated from precursor T cells obtained from one or more suitable donor or stem cells such as hematopoietic stem cells or inducible pluripotent stem cells (iPSC), which may be cultured in vitro.

In some embodiments, the genetically engineered T cells carry a disrupted CD83 gene. Such genetically engineered T cells may further comprise one or more disrupted genes, for example, TRAC and/or β2M. Such genetically engineered T cells may further express a chimeric antigen receptor (CAR), which may be capable of binding to an antigen of interest, for example, CD83.

Any of the genetically engineered T cells may be generated via gene editing (including genomic editing), a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

(a) Genetically Edited Genes

In some aspects, the present disclosure provides genetically engineered T cells that may comprise a disrupted CD83 gene. In some instances, the genetically engineered T cells disclosed herein may further a disrupted 2M gene, a disrupted TRAC gene, a disrupted CD70 gene, or a combination thereof. In specific examples, the genetically engineered T cells disclosed herein may further a disrupted β2M gene and a disrupted TRAC gene.

As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

CD83 Gene Editing

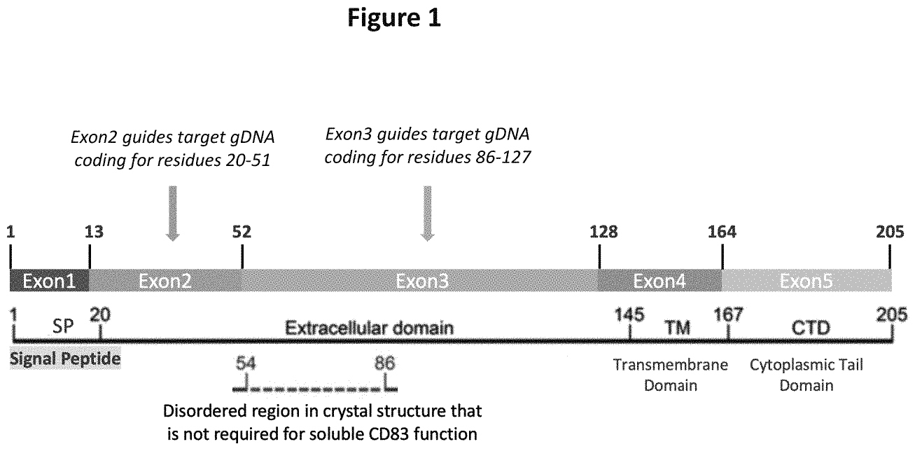

In some embodiments, the genetically engineered T cells may comprise a disrupted CD83 gene. CD83 is a member of the immunoglobulin (Ig) superfamily and is expressed in membrane bound or soluble forms. The membrane-bound CD83 contains an extracellular V-type immunoglobulin-like domain, a transmembrane domain and a cytoplasmic signaling domain. The soluble form contains only the −type immunoglobulin-like domain. The gene encoding CD83 is located on human chromosome 6p23. The structure of the human CD83 gene is known in the art, e.g., under Gene ID ENSG00000112149. See also FIG. 1.

CD83 is expressed in various types of immune cells, including regulatory T cells, dendritic cells, B cells, and T cells. It was reported that CD83 may involve in inflammation and serves as a binding site for the aryl hydrocarbon receptor.

In some examples, the genetically engineered T cells may comprise a disrupted CD83 gene such that the expression of CD83 in the T cells is substantially reduced or eliminated completely. The disrupted CD83 gene may comprise one or more genetic edits at one or more suitable target sites (e.g., in coding regions or in non-coding regulatory regions such as promoter regions) that disrupt expression of the CD83 gene. Such target sites may be identified based on the gene editing approach for use in making the genetically engineered T cells. Exemplary target sites for the genetic edits may include exon 2, exon 3, or a combination thereof. In some examples, one or more genetic editing may occur in exon 2. In other examples, one or more genetic editing may occur in exon 3. Such genetic editing may be induced by the CRISPR/Cas technology using a suitable guide RNA, for example, those listed in Table 2.

β2M Gene Edit

In some embodiments, the genetically engineered T cells disclosed herein may further comprise a disrupted β2M gene. (32M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

In some embodiments, the gRNA targeting β2M listed in Table 1 (B2M1 or B2M4) may be used for disrupting the β2M gene via CRISPR/Cas9 gene editing.

In some embodiments, an edited β2M gene may comprise a nucleotide sequence selected from the following sequences in Table 4. It is known to those skilled in the art that different nucleotide sequences in an edited gene such as an edited β2M gene (e.g., those in Table 4) may be generated by a single gRNA such as the ones listed in Table 1 (e.g., B2M1). See also WO2019097305, the relevant disclosures of which are incorporated by reference for the subject matter and purpose referenced herein.

The genetically engineered T cells disclosed herein may further comprise one or more additional gene edits (e.g., gene knock-in or knock-out) to improve T cell function. Examples include knock-in or knock-out genes to improve target cell lysis, knock-in or knock-out genes to enhance performance of therapeutic T cells such as CAR-T cells prepared from the genetically engineered T cells.

TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein.

In some embodiments, the gRNA targeting TRAC listed in Table 1 (TA-1) may be used for disrupting the TRAC gene via CRISPR/Cas9 gene editing.

In some embodiments, an edited TRAC gene may comprise a nucleotide sequence selected from the following sequences in Table 3. It is known to those skilled in the art that different nucleotide sequences in an edited gene such as an edited TRAC gene (e.g., those in Table 3) may be generated by a single gRNA such as the one listed in Table 1 (TA-1).

It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.

CD70 Gene Editing

T cell exhaustion is a process of stepwise and progressive loss of T cell functions, which may be induced by prolonged antigen stimulation or other factors. Genes involved in T cell exhaustion refer to those that either positively regulate or negatively regulate this biological process. The genetically engineered T cells disclosed herein may comprise genetic editing of a gene that positively regulates T cell exhaustion to disrupt its expression. Alternatively or in addition, the genetically engineered T cells may comprise genetic editing of a gene that negatively regulates T cell exhaustion to enhance its expression and/or biologic activity of the gene product.

In some embodiments, the genetically engineered T cells may comprise an edited gene involved in T cell exhaustion, e.g., disruption of a gene that positively regulates T cell exhaustion. Such a gene may be a Cluster of Differentiation 70 (CD70) gene. CD70 is a member of the tumor necrosis factor superfamily and its expression is restricted to activated T and B lymphocytes and mature dendritic cells. CD70 is implicated in tumor cell and regulatory T cell survival through interaction with its ligand, CD27. CD70 and its receptor CD27 have multiple roles in immune function in multiple cell types including T cells (activated and T_{reg} cells), and B cells.

It was also found that disrupting the CD70 gene in immune cells engineered to express an antigen targeting moiety enhanced anti-tumor efficacy against large tumors and induced a durable anti-cancer memory response. Specifically, the anti-cancer memory response prevented tumor growth upon re-challenge. Further, it has been demonstrated disrupting the CD70 gene results in enhanced cytotoxicity of immune cells engineered to express an antigen targeting moiety at lower ratios of engineered immune cells to target cells, indicating the potential efficacy of low doses of engineered immune cells. See, e.g., WO2019/215500, the relevant disclosures of which are incorporated by reference for the purpose and subject matter referenced herein.

Structures of CD70 genes are known in the art. For example, human CD70 gene is located on chromosome 19p13.3. The gene contains four protein encoding exons. Additional information can be found in GenBank under Gene ID: 970.

In some examples, the genetically engineered T cells may comprise a disrupted CD70 gene such that the expression of CD70 in the T cells is substantially reduced or eliminated completely. The disrupted CD70 gene may comprise one or more genetic edits at one or more suitable target sites (e.g., in coding regions or in non-coding regulatory regions such as promoter regions) that disrupt expression of the CD70 gene. Such target sites may be identified based on the gene editing approach for use in making the genetically engineered T cells. Exemplary target sites for the genetic edits may include exon 1, exon 2, exon 3, exon 4, or a combination thereof. See also WO2019/215500, the relevant disclosures of which are incorporated by reference for the purpose and subject matter referenced herein.

In some embodiments, the gRNA targeting CD70 listed in Table 1 (CD70-T7) may be used for disrupting the CD70 gene via CRISPR/Cas9 gene editing.

(b) Methods of Making Genetically Engineered T Cells

The genetically engineered T cells disclosed herein can be prepared by genetic editing of parent T cells or precursor cells thereof via a conventional gene editing method or those described herein.

(a) T Cells Sources

In some embodiments, T cells can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRαβ, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRαβ, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some instances, the T cell population comprises primary T cells isolated from one or more human donors. Such T cells are terminally differentiated, not transformed, depend on cytokines and/or growth factors for growth, and/or have stable genomes.

Alternatively, the T cells may be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

(b) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95:e52118).

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

Some exemplary approaches are disclosed in detail below.

(i) CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

Endonuclease for use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease).

Provided below is the amino acid sequence of an exemplary Cas9 enzyme of S. pyogenes:

(SEQ ID NO: 1)
MDKKYSIGLDIGTNSVGWAVITDEYKVPSKKFKVLGNTDRHSIKKNLIGALLFDSGETAEATRLK
RTARRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAE
DAKLQLSKDTYDDDLDNLLAQIGDQYADLFLAAKNLSDAILLSDILRVNTEITKAPLSASMIKRY
DEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQS
FLKDDSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRIDLSQL
GGD

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence ranges from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO: 11), then the gRNA spacer sequence is 5′-AGAGCAACAGUGCUGUGGCC**-3′ (SEQ ID NO: 4). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20-nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 1 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a CD83 gene, for example, target a site within exon 1, exon 2, exon 3, or exon 4 of the CD83 gene. In some examples, the gRNA may target a site within exon 2 of the CD83 gene. Such a gRNA may comprise a spacer sequence complementary (complete or partially) to the target sequences in the target exon (e.g., exon 1, exon 2, exon 3, or exon 4) of a CD83 gene, or a fragment thereof. Exemplary target sequences in a CD83 gene and exemplary gRNA sequences are provided in Table 2 below, all of which are within the scope of the present disclosure.

In some examples, the gRNA comprises a spacer targeting the CD83 site that comprises SEQ ID NO: 74 (e.g., guide CD83-G2, a.k.a., CD83-2). Such a spacer may comprise the nucleotide sequence of SEQ ID NO: 105. In one example, the spacer consists of SEQ ID NO: 105. Such gRNAs may further comprise a scaffold sequence such as SEQ ID NO: 155 or SEQ ID NO: 234.

In other examples, the gRNA comprises a spacer targeting the CD83 site that comprises SEQ ID NO: 88 (e.g., guide CD83-G9, a.k.a., CD83-9). Such a spacer may comprise the nucleotide sequence of SEQ ID NO: 133. In one example, the spacer consists of SEQ ID NO: 133. Such gRNAs may further comprise a scaffold sequence such as SEQ ID NO: 155 or SEQ ID NO: 234.

In yet other examples, the gRNA comprises a spacer targeting the CD83 site that comprises SEQ ID NO: 169 (e.g., guide CD83-17). Such a spacer may comprise the nucleotide sequence of SEQ ID NO: 176. In one example, the spacer consists of SEQ ID NO: 176. Such gRNAs may further comprise a scaffold sequence such as SEQ ID NO: 155 or SEQ ID NO: 234.

In some embodiments, the gRNAs disclosed herein target a β2M gene, for example, target a suitable site within a β2M gene. See also WO2019097305, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In some embodiments, the gRNAs disclosed herein target a TRAC gene. See also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein.

Exemplary spacer sequences and gRNAs targeting a β2M gene or TRAC gene are provided in Table 1 below. See also WO2019097305, the relevant disclosures of which are incorporated by reference herein for the purpose and subject matter referenced herein.

In some embodiments, the gRNAs disclosed herein target a CD70 gene, for example, target a site within exon 1 or exon 3 of a CD70 gene. Such a gRNA may comprise a spacer sequence complementary (complete or partially) to the target sequences in exon 1 or exon 3 of a CD70 gene, or a fragment thereof. Exemplary target sequences in a CD70 gene and exemplary gRNAs specific to the CD70 gene are provided in Table 1 below.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments of the present disclosure, a CRISPR/Cas nuclease system for use in genetically editing any of the target genes disclosed here may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and a RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively, or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used.

(ii) Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and WP/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

### II. Genetically Engineered T Cells Expression a Chimeric Antigen Receptor (CAR)

The genetically engineered T cells having a disrupted CD83 gene and optionally one or more of additional disrupted genes, e.g., β2M, TRAC, or a combination thereof as disclosed herein, may further express a chimeric antigen receptor (CAR) targeting an antigen of interest or cells expressing such an antigen.

(a) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include SEQ ID NO: 53 and SEQ ID NO: 54 as provided in Table 5 below. Other signal peptides may be used.

(i) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen that is expressed on undesired cells, for example, tumor cells or undesired immune cells (e.g., alloreactive cells or autoreactive cells). In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

In some embodiments, the antigen-binding extracellular domain can be a single-chain variable fragment (scFv) that binds a tumor antigen as disclosed herein, for example, CD19, BCMA, or CD70. The scFv may comprise an antibody heavy chain variable region (V_{H}) and an antibody light chain variable region (V_{L}), which optionally may be connected via a flexible peptide linker. In some instances, the scFv may have the V_{H} to V_{L} orientation (from N-terminus to C-terminus). Alternatively, the scFv may have the V_{L} to V_{H} orientation (from N-terminus to C-terminus).

In some examples, the pathologic antigen (e.g., an antigen of interest) may be a cell surface receptor expressed on immune cells, for example, autoreactive immune cells or alloreactive immune cells. In one specific example, the antigen of interest is CD83.

In some examples, the antigen-binding extracellular domain can be a single-chain variable fragment (scFv) that binds human CD83. In some instances, the anti-CD83 scFv may comprises (i) a heavy chain variable region (V_{H}) that comprises the same heavy chain complementary determining regions (CDRs) as those in SEQ ID NO: 49; and (ii) a light chain variable region (V_{L}) that comprises the same light chain CDRs as those in SEQ ID NO: 50. In some specific examples, the anti-CD83 antibody discloses herein may comprise the heavy chain CDR1, heavy chain CDR2, and heavy chain CDR3 set forth as SEQ ID NOs: 43-45, respectively as determined by the Kabat method. Alternatively, or in addition, the anti-CD83 antibody discloses herein may comprise the light chain CDR1, light chain CDR2, and light chain CDR3 set forth as SEQ ID NOs: 46-48 as determined by the Kabat method. In one specific example, the anti-CD83 scFv may comprise a V_{H} comprising the amino acid sequence of SEQ ID NO: 49 and a V_{L} comprises the amino acid sequence of SEQ ID NO: 50. See Sequence Table 5 below.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/ or abysis.org/abysis/sequence_input).

(ii) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of SEQ ID NO: 58 as provided below in Table 5. Other transmembrane domains may be used.

(iii) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used. See Table 5 below for examples.

(iv) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 5 provides examples of signaling domains derived from 4-1BB, CD28 and CD3-zeta that may be used herein.

In specific examples, the anti-CD83 CAR disclosed herein may comprise the amino acid sequence of SEQ ID NO: 52, which may be encoded by the nucleotide sequence of SEQ ID NO: 66. See sequence Tables 5 and 6 provided below.

(b) Delivery of CAR Construct to T Cells

In some embodiments, a nucleic acid encoding a CAR can be introduced into any of the genetically engineered T cells disclosed herein by methods known to those of skill in the art. For example, a coding sequence of the CAR may be cloned into a vector, which may be introduced into the genetically engineered T cells for expression of the CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In specific examples, a nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a gene of interest to disrupt expression of the gene of interest. In some instances, the viral vector such as an adeno-associated viral (AAV) vector) can be designed such that it can insert into a location within the TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. See Table 1 below. Any of the gRNAs specific to a TRAC gene and the target regions disclosed herein can be used for this purpose.

In some embodiments, a disrupted gene of interest may comprise a deletion of a fragment, which may be the target site of a guide RNA used for making the disrupted gene. In some instances, the deleted fragment may be replaced by a donor template comprising the nucleotide sequence coding for the CAR polypeptide.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting a CAR coding segment into the TRAC gene.

In some embodiments, a nucleic acid encoding a CAR (e.g., via a donor template, which can be carried by a viral vector such as an adeno-associated viral (AAV) vector) can be designed such that it can insert into a location within a β2M gene to disrupt the β2M gene in the genetically engineered T cells and express the CAR polypeptide. Disruption of 32M leads to loss of function of the endogenous MHC Class I complexes. For example, a disruption in the β2M gene can be created with an endonuclease such as those described herein and one or more gRNAs targeting one or more β2M genomic regions. Any of the gRNAs specific to a β2M gene and the target regions disclosed herein can be used for this purpose.

In some examples, a genomic deletion in the β2M gene and replacement by a CAR coding segment can be created by homology directed repair or HDR (e.g., using a donor template, which may be part of a viral vector such as an adeno-associated viral (AAV) vector). In some embodiments, a disruption in the β2M gene can be created with an endonuclease as those disclosed herein and one or more gRNAs targeting one or more β2M genomic regions and inserting a CAR coding segment into the β2M gene.

In some embodiments, a nucleic acid encoding a CAR (e.g., via a donor template, which can be carried by a viral vector such as an adeno-associated viral (AAV) vector) can be designed such that it can insert into a location within a CD83 gene to disrupt the CD83 gene in the genetically engineered T cells and express the CAR polypeptide. Disruption of CD83 leads to loss of function of the endogenous CD83 protein. For example, a disruption in the CD83 gene can be created with an endonuclease such as those described herein and one or more gRNAs targeting one or more CD83 genomic regions. Any of the gRNAs specific to a CD83 gene and the target regions disclosed herein can be used for this purpose.

In some examples, a genomic deletion in the CD83 gene and replacement by a CAR coding segment can be created by homology directed repair or HDR (e.g., using a donor template, which may be part of a viral vector such as an adeno-associated viral (AAV) vector). In some embodiments, a disruption in the CD83 gene can be created with an endonuclease as those disclosed herein and one or more gRNAs targeting one or more Reg1 genomic regions and inserting a CAR coding segment into the CD83 gene.

A donor template as disclosed herein can contain a coding sequence for a CAR. In some examples, the CAR-coding sequence may be flanked by two regions of homology to allow for efficient HDR at a genomic location of interest, for example, at a TRAC gene, at a β2M gene, or at a CD83 gene, using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene, the β2M gene, the CD70 gene, or the CD83 gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous prompter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO: 65 provided in Table 6 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

When needed, additional gene editing (e.g., gene knock-in or knock-out) can be introduced into therapeutic T cells as disclosed herein to improve T cell function and therapeutic efficacy. For example, β2M disruption can be performed to reduce the risk of or prevent a host-versus-graft response. Other examples include knock-in or knock-out genes to improve target cell lysis, knock-in or knock-out genes to enhance performance of therapeutic T cells such as CAR-T cells.

In some examples, a donor template for delivering an anti-CD83 CAR may be an AAV vector inserted with a nucleic acid fragment comprising the coding sequence of the anti-CD83 CAR, and optionally regulatory sequences for expression of the anti-CD83 CAR (e.g., a promoter such as the EF1α promoter provided in Table 6 below), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 10. In some specific examples, the donor template for delivering the anti-CD83 CAR may comprise a nucleotide sequence of SEQ ID NO: 66, which can be inserted into a disrupted TRAC gene, for example, replacing the fragment of SEQ ID NO: 10.

The genetically engineered T cells having a disrupted CD83 gene, additional disrupted genes, e.g., 82M, TRAC, and/or CD70 and further expressing a chimeric antigen receptor (CAR) can be produced by sequential targeting of the genes of interest. For example, in some embodiments, the CD83 gene may be disrupted first, followed by disruption of TRAC and/or β2M genes and CAR insertion. In other embodiments, TRAC and/or β2M genes may be disrupted first, followed by CAR insertion and disruption of the CD83 gene. Accordingly, in some embodiments, the genetically engineered T cells disclosed herein may be produced by multiple, sequential electroporation events with multiple RNPs targeting the genes of interest, e.g., CD83 and optionally, β2M, TRAC and/or CD70.

In other embodiments, the genetically engineered CAR T cells disclosed herein may be produced by a single electroporation event with an RNP complex comprising an RNA-guided nuclease and multiple gRNAs targeting the genes of interest, e.g., CD83 and optionally, β2M, and/or TRAC.

(c) Exemplary Genetically Engineered Immune Cells Expression a Chimeric Antigen Receptor

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a CD83 gene edit may be considered a CD83 knockout cell if the CD83 protein cannot be detected at the cell surface using an antibody that specifically binds the CD83 protein. Similarly, a cell having a β2M gene edit may be considered a β2M knockout cell if (32M protein cannot be detected at the cell surface using an antibody that specifically binds (32M protein.

In some embodiments, a population of genetically engineered immune cells such as T cells disclosed herein express a CAR (e.g., anti-CD83 CAR), a disrupted CD83 gene, and optionally a disrupted TRAC gene, a disrupted β2M gene, a disrupted CD70 gene, or a combination thereof (e.g., a disrupted TRAC gene and a disrupted β2M gene). The nucleotide sequence encoding the CAR may be inserted in a genomic site of interest, for example, in the disrupted TRAC gene (e.g., replacing the site targeted by a sgRNA such as TA-1 provided in Table 1 below), in the disrupted β2M gene (replacing the site targeted by a sgRNA such as B2M1 provided in Table 1 below), or in the disrupted CD83 gene (replacing the site targeted by a sgRNA such as those provided in Table 2 below). In some instances, the gRNA targeting the CD83 gene is CD83-2 (G2) or a gRNA targeting the same site in CD83 as CD83-2. In some instances, the gRNA targeting the CD83 gene is CD83-9 (G9) or a gRNA targeting the same site in CD83 as CD83-9. In some instances, the gRNA targeting the CD83 gene is CD83-17 or a gRNA targeting the same site in CD83 as CD83-17.

In some examples, such a population of genetically engineered T cells may comprise at least 50% CD83⁻ cells, for example, at least 60%, at least 70%, at least 80%, at least 90% or above, CD83⁻ cells. Alternatively or in addition, the population of genetically engineered T cells may comprise at least 50% TCR⁻ cells, for example, at least 60%, at least 70%, at least 80%, at least 90% or above, TCR⁻ cells. Alternatively or in addition, the population of genetically engineered T cells may comprise at least 50% (32M⁻ cells, for example, at least 60%, at least 70%, at least 80%, at least 90% or above, (32M⁻ cells. Alternatively or in addition, the population of genetically engineered T cells may comprise at least 50% CD70⁻ cells, for example, at least 60%, at least 70%, at least 80%, at least 90% or above, CD70⁻ cells. In some instances, the population of genetically engineered T cells may comprise at least 40% CAR+ cells (e.g., anti-CD83 CAR+ cells), for example, at least 50%, at least 60%, at least 70%, at least 80%, at least 90% or above, CAR+ cells.

In some examples, the population of genetically engineered T cells may comprise at least 50% of the engineered T cells expressing a detectable level of the CAR (e.g., an anti-CD83 CAR) and does not express a detectable level of CD83 on cell surface.

In some examples, the genetically engineered immune cells such as T cells disclosed herein do not proliferate or cause cell lysis in the absence of stimulation by cytokine, growth factor, and/or antigen. In other examples, the genetically engineered immune cells such as T cells, when co-cultured with CD83+ cells, would result in lysis of at least 50% (e.g., at least 55%, at least 60%, at least 65%, at least 70%, at least 75% or higher) of the CD83+ cells.

In some examples, the genetically engineered immune cells such as T cells disclosed herein, carrying a disrupted CD83 gene, have an expansion capacity at least 50% higher (e.g., at least 1-fold higher, at least 2-fold higher, at least 3-fold higher, at least 4-fold higher, or above) relative to counterpart immune cells having a wild-type CD83 gene. Alternatively or in addition, the genetically engineered immune cells such as T cells disclosed herein, carrying a disrupted CD83 gene, have reduced production of pro-inflammatory cytokines (e.g., IL-2, TNFα, and/or IFNγ), for example, at least 30% lower (e.g., at least 40% lower, at least 50% lower, at least 80% lower, at least 1-fold lower, or at least 2-fold lower) relative to the counterpart immune cells having a wild-type CD83 gene. Further, the genetically engineered immune cells such as T cells disclosed herein, carrying a disrupted CD83 gene, may have increased CD8+ cell frequency when cultured in vitro, as compared with the counterpart immune cells having a wild-type CD83 gene cultured under the same conditions.

In some examples, the population of genetically engineered immune cells such as T cells disclosed herein express an anti-CD83 CAR, a disrupted CD83 gene, a disrupted TRAC gene and a disrupted β2M gene. Such a population of genetically engineered T cells can be prepared using immune cells from one or more healthy human donors.

The anti-CD83 CAR comprises one or more of the components listed in Table 5 below. In one example, the anti-CD83 CAR comprises a VH set forth as SEQ ID NO: 49 and a VL set forth as SEQ ID NO:50. Such an anti-CD83 CAR may comprise a scFv set forth as SEQ ID NO: 51. In one specific example, the anti-CD83 CAR comprises the amino acid sequence of SEQ ID NO: 235 or SEQ ID NO: 52. The anti-CD83 CAR encoding sequence can be inserted in the disrupted TRAC locus. In some instances, the disrupted TRAC locus comprises the nucleotide sequence of SEQ ID NO: 66 (in which the CAR coding sequence replaces SEQ ID NO: 11 of the TRAC target site). The disrupted CD83 gene is generated using gRNA CD83-2 (G2) via the CRISPR/Cas-mediated gene editing system. The disrupted β2M gene can be generated using either the B2M1 guide or the B2M4 guide via the CRISPR/Cas-mediated gene editing system.

In one example, the population of genetically engineered immune cells (e.g., T cells) disclosed herein comprises (a) a disrupted TRAC gene, which is genetically edited at SEQ ID NO: 11 within the TRAC locus, (b) a disrupted β2M gene, which is genetically edited at SEQ ID NO: 13 within the β2M locus, (c) a disrupted CD83 gene, which is genetically edited at SEQ ID NO: 74 within the CD83 locus, and (d) a nucleic acid encoding an anti-CD83 CAR, which is inserted into the disrupted TRAC gene (e.g., at SEQ ID NO: 11). The anti-CD83 CAR comprise a V_{H} set forth as SEQ ID NO: 49 and a V_{L} set forth as SEQ ID NO: 50. Such an anti-CD83 CAR may comprise an anti-CD83 scFv set forth as SEQ ID NO: 51. In one specific example, the anti-CD83 CAR comprises the amino acid sequence of SEQ ID NO: 52 (with signal peptide) or SEQ ID NO: 235 (without signal peptide). The disrupted TRAC may comprise the nucleotide sequence of SEQ ID NO: 66 (in which SEQ ID NO: 11 is replaced with the nucleic acid encoding the anti-CD83 CAR). In some instances, the disrupted TRAC gene is produced with a gRNA targeting SEQ ID NO:11 (e.g., TA-1 listed in Table 1) via CRISPR/Cas9-mediated gene editing, the disrupted β2M gene is produced with a gRNA targeting SEQ ID NO: 13 (e.g., B2M1 listed in Table 1) via CRISPR/Cas9-mediated gene editing, the disrupted CD83 gene is produced with a gRNA targeting SEQ ID NO: 74 (e.g., CD83-2 listed in Table 2) via CRISPR/Cas9-mediated gene editing, or a combination thereof.

In another example, the population of genetically engineered immune cells (e.g., T cells) disclosed herein comprises (a) a disrupted TRAC gene, which is genetically edited at SEQ ID NO: 11 within the TRAC locus, (b) a disrupted β2M gene, which is genetically edited at SEQ ID NO: 231 within the β2M locus, (c) a disrupted CD83 gene, which is genetically edited at SEQ ID NO: 74 within the CD83 locus, and (d) a nucleic acid encoding an anti-CD83 CAR, which is inserted into the disrupted TRAC gene (e.g., at SEQ ID NO: 11). The anti-CD83 CAR comprise a V_{H} Set forth as SEQ ID NO: 49 and a V_{L} set forth as SEQ ID NO: 50. Such an anti-CD83 CAR may comprise an anti-CD83 scFv set forth as SEQ ID NO: 51. In one specific example, the anti-CD83 CAR comprises the amino acid sequence of SEQ ID NO: 52 (with signal peptide) or SEQ ID NO: 235 (without signal peptide). The disrupted TRAC may comprise the nucleotide sequence of SEQ ID NO: 66 (in which SEQ ID NO: 11 is replaced with the nucleic acid encoding the anti-CD83 CAR). In some instances, the disrupted TRAC gene is produced with a gRNA targeting SEQ ID NO:11 (e.g., TA-1 listed in Table 1) via CRISPR/Cas9-mediated gene editing, the disrupted β2M gene is produced with a gRNA targeting SEQ ID NO: 231 (e.g., B2M4 listed in Table 1) via CRISPR/Cas9-mediated gene editing, the disrupted CD83 gene is produced with a gRNA targeting SEQ ID NO: 74 (e.g., CD83-2 listed in Table 2) via CRISPR/Cas9-mediated gene editing, or a combination thereof.

### III. Therapeutic Applications

The genetically engineered immune cells such as T cells can be used to eliminate undesired cells (e.g., pathological cells such as cancer cells or undesired immune cells) and treat diseases associated with such undesired cells, e.g., cancer or autoimmune diseases. In some instances, the undesired disease cells are CD83+ cell. Given the superior features of the genetically engineered immune cells having a disrupted CD83 gene relative to counterpart cells having a wild-type CD83 gene, the genetically engineered immune cells as disclosed herein would be expected to be advantageous in both manufacturing and therapeutic applications.

Any of the genetically engineered immune cells (e.g., T cells) having a disrupted CD83 gene and optionally a disrupted TRAC, β2M, and/or CD70 genes and expressing a CAR (e.g., an anti-CD83 CAR) can be administered to a subject for therapeutic purposes, for example, treatment of disease associated with CD83+ disease cells (e.g., cancer or an immune disease such as autoimmune disease). Exemplary immune diseases include lupus and chronic and acute GvHD.

Exemplary cancer may be a hematopoietic cancer, for example, AML, CD19+ Leukemia, or CD19+ lymphomas. In some instances, a second population of CAR-T cells maybe co-used with the engineered immune cells having a disrupted CD83 gene as disclosed herein. For example, anti-CD19 CAR-T cells may be co-used with the CD83 disrupted immune cells for treating diseases involving CD19+ cells, such as AML, CD19+ Leukemia, or CD19+ lymphomas.

The step of administering may include the placement (e.g., transplantation) of the genetically engineered immune cells such as T cells into a subject by a method or route that results in at least partial localization of the engineered immune cells at a desired site such that a desired effect(s) can be produced. The genetically engineered immune cells such as T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects as described herein, an effective amount of the therapeutic T cells can be administered via a systemic route of administration, such as an intraperitoneal or intravenous route.

In some embodiments, the genetically engineered immune cells such as T cells can be administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

A subject may be any subject for whom diagnosis, treatment, or therapy is desired. In some embodiments, the subject is a mammal. In some embodiments, the subject is a human.

In some instances, the genetically engineered immune cells such as T cells may be autologous (“self”) to the subject, i.e., the cells are from the same subject. Alternatively, the therapeutic T cells can be non-autologous (“non-self,” e.g., allogeneic, syngeneic or xenogeneic) to the subject. “Allogeneic” means that the therapeutic T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used.

An effective amount refers to the amount of a population of engineered immune cells such as T cells needed to prevent or alleviate at least one or more signs or symptoms of a medical condition (e.g., cancer or immune disorder), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some examples, an effective amount of the genetically engineered immune cells such as T cells disclosed herein may be less than 10⁶ cells (e.g., CAR+ cells), e.g., 10⁵ cells, 5×10⁴ cells, 10⁴ cells, 5×10³ cells, or 10³ cells. In other examples, an effective amount of the genetically engineered immune cells such as T cells disclosed herein may be greater than 10⁶ cells (e.g., CAR+ cells), for example, 10⁷ cells, 10⁸ cells, or 10⁹ cells. In some examples described herein, the cells are expanded in culture prior to administration to a subject in need thereof.

The efficacy of a treatment using the therapeutic T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the genetically engineered T cells disclosed herein may express an anti-CD83 CAR (e.g., those disclosed herein; see Tables 5 and 6 below) and can be used for eliminating undesired cells that are CD83+. In some examples, the undesired cells are cancer cells (e.g., CD83+ cancer cells). The genetically engineered T cells expressing an anti-CD83 CAR can be used for treating a CD83+ cancer. In other examples, the undesired cells are immune cells (e.g., CD83+ B cells or CD83+ dendritic cells). In some instances, the genetically engineered T cells expressing an anti-CD83 CAR can be used for treating an immune disorder, e.g., those in which the CD83+ immune cells play a role. The immune disorder may be an autoimmune disease, sepsis, rheumatological disease, diabetes, or asthma.

In some examples, the target disease can be a B cell mediated autoimmune disease. Examples include Examples include, but are not limited to, Achalasia, Acute disseminated encephalomyelitis (ADEM), Addison's disease, Adiposis dolorosa, Adult Still's disease, Agammaglobulinemia, Alopecia areata, Amyloidosis, Ankylosing spondylitis, Anti-GBM/Anti-TBM nephritis, Anti-N-Methyl-D-Aspartate (Anti-NMDA) receptor encephalitis, Antiphospholipid syndrome, Antisynthetase syndrome, Aplastic Anemia, Autoimmune angioedema, Autoimmune dysautonomia, Autoimmune encephalomyelitis, Autoimmune enteropathy, Autoimmune hepatitis, Autoimmune inner ear disease (AIED), Autoimmune lymphoproliferative syndrome, Autoimmune myocarditis, Autoimmune oophoritis, Autoimmune orchitis, Autoimmune pancreatitis, Autoimmune polyendocrine syndrome (APS) type 1, Autoimmune polyendocrine syndrome (APS) type 2, Autoimmune polyendocrine syndrome (APS) type 3, Autoimmune retinopathy, Autoimmune urticaria, Axonal & neuronal neuropathy (AMAN), Baló disease, Behcet's disease, Benign mucosal pemphigoid, Bickerstaff's encephalitis, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Drug-induced lupus, Endometriosis, Enthesitis-related arthritis, Eosinophilic esophagitis (EoE), Eosinophilic fasciitis, Epidermolysis bullosa acquisita, Erythema nodosum, Essential mixed cryoglobulinemia, Evans syndrome, Felty Syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hasimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgA Vasculitis, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lupus Nephritis, Lupus Vasculitis, Lyme disease chronic, Meniere's disease, Microscopic polyangiitis (MPA), Mixed connective tissue disease (MCTD), Mooren's ulcer, Morphea, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neuromyotonia, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), Pityriasis lichenoides et varioliforis acuta, POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cholangitis, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham's chorea, Sympathetic ophthalmia (SO), Systemic lupus erythematosus (SLE), Takayasu's arteritis, Temporal arteritis/Giant cell arteritis, Thrombocytopenia, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Urticarial vasculitis, Uveitis, Vasculitis, Vitiligo, and Vogt-Koyanagi-Harada Disease.

Combination therapies are also encompassed by the present disclosure. For example, the genetically engineered immune cells such as T cells disclosed herein may be co-used with other therapeutic agents, for treating the same indication, or for enhancing efficacy of the therapeutic T cells and/or reducing side effects of the therapeutic T cells. In some instances, the other therapeutic agent may comprise cell-based therapeutics such as CAR-T cells, which may target an antigen of interest that is different from CD83 (e.g., a tumor antigen such as CD19, BCMA, or CD70). The genetically engineered immune cells disclosed herein, having a disrupted CD83 gene and expressing an anti-CD83 CAR, may eliminate immune cells reactive to the cell-based therapeutics (e.g., alloreactive immune cells), thereby enhancing therapeutic efficacy by the cell-based therapeutics.

In some embodiments, the genetically engineered immune cells (e.g., T cells), having a disrupted CD83 gene, may express both an anti-CD83 CAR and a CAR targeting a tumor antigen such as those disclosed herein. Such genetically engineered immune cells such as T cells may further comprise a disrupted TRAC gene, a β2M gene, or both.

### IV. Kits

The present disclosure also provides kits for use in producing the genetically engineered immune cells, such as T cells, and for therapeutic uses.

In some embodiments, a kit provided herein may comprise components for performing genetic edit of CD83 gene, and optionally TRAC and/or β2M, and for introducing a CAR construct. The kit may further comprise a population of immune cells to which the genetic editing is to be performed (e.g., a leukopak). A leukopak sample may be an enriched leukapheresis product collected from peripheral blood. It typically contains a variety of blood cells including monocytes, lymphocytes, platelets, plasma, and red cells. The components for genetically editing one or more of the target genes may comprise a suitable endonuclease such as an RNA-guided endonuclease and one or more nucleic acid guides, which direct cleavage of one or more suitable genomic sites by the endonuclease. For example, the kit may comprise a Cas enzyme such as Cas 9 and one or more gRNAs targeting a CD83 gene. Such a kit may further comprise components for further gene editing, for example, gRNAs and optionally additional endonucleases for editing other target genes such as β2M and/or TRAC. Such components may further comprise an endonuclease suitable for gene editing and a nucleic acid coding for a CAR construct of interest. The CAR-coding nucleic acid may be part of a donor template as disclosed herein, which may contain homologous arms flanking the CAR-coding sequence. In some instances, the donor template may be carried by a viral vector such as an AAV vector.

In some embodiments, the kit may comprise one or more gRNAs specific to a gene of interest for inserting the CAR-coding sequence into the gene of interest. For example, the kit may comprise gRNAs specific to a TRAC gene for inserting the CAR-coding sequence into the TRAC gene. In other examples, the kit may further comprise gRNAs specific to a 2M gene for inserting the CAR-coding sequence into the 2M gene. In other examples, the kit may further comprise gRNAs specific to a CD83 gene for inserting the CAR-coding sequence into the CD83 gene.

In yet other embodiments, the kit disclosed herein may comprise a population of any of the genetically engineered immune cells such as T cells as disclosed for the intended therapeutic purposes.

Any of the kit disclosed herein may further comprise instructions for making the genetically engineered immune cells and/or instructions for therapeutic applications of the engineered cells. In some examples, the included instructions may comprise a description of using the gene editing components to genetically engineer one or more of the target genes (e.g., CD83, and optionally TRAC and/or β2M). In other examples, the included instructions may comprise a description of how to introduce a nucleic acid encoding a CAR construction into the T cells for making therapeutic T cells.

Alternatively, the kit may further comprise instructions for administration of the genetically engineered immune cells such as T cells as disclosed herein to achieve the intended activity, e.g., eliminating disease cells targeted by the CAR expressed on the engineered T cells. The kit may further comprise a description of selecting a subject suitable for treatment based on identifying whether the subject is in need of the treatment. The instructions relating to the use of the therapeutic T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the therapeutic T cells are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the therapeutic T cells. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Sequence Tables

TABLE 1
gRNA Sequences

| Name | Unmodified Sequence | Modified Sequence |
|---|---|---|
| TRAC sgRNA | AGAGCAACAGUGCUGU | A*G*A*GCAACAGUGCU |
| (TA-1) | GGCCguuuuagagcuagaaauag | GUGGCCguuuuagagcuagaaa |
| | caaguuaaaauaaggcuaguccguu | uagcaaguuaaaauaaggcuagucc |
| | aucaacuugaaaaaguggcaccgag | guuaucaacuugaaaaaguggcacc |
| | ucggugcUUUU | gagucggugcU*U*U*U |
| | (SEQ ID NO: 2) | (SEQ ID NO: 3) |
| TRAC sgRNA spacer | AGAGCAACAGUGCUGU | A*G*A*GCAACAGUGCU |
| (TA-1) | GGCC (SEQ ID NO: 4) | GUGGCC (SEQ ID NO: 5) |
| β2M sgRNA | GCUACUCUCUCUUUCUG | G*c*u*acucucucuuuc |
| (B2M1) | GCCguuuuagagcuagaaauagca | UGGCCguuuuagagcuagaaaua |
| | aguuaaaauaaggcuaguccguuau | gcaaguuaaaauaaggcuaguccgu |
| | caacuugaaaaaguggcaccgaguc | uaucaacuugaaaaaguggcaccga |
| | ggugcUUUU (SEQ ID NO: | gucggugcU*U*U*U (SEQ ID |
| | 6) | NO: 7) |
| β2M sgRNA spacer | GCUACUCUCUCUUUCUG | G*C*U*ACUCUCUCUUUC |
| (B2M1 spacer) | GCC (SEQ ID NO: 8) | UGGCC (SEQ ID NO: 9) |
| β2M sgRNA | CAGUAAGUCAACUUCAA | C*A*G*UAAGUCAACUU |
| (B2M4) | UGUguuuuagagcuagaaauagc | CAAUGUguuuuagagcuagaaa |
| | aaguuaaaauaaggcuaguccguua | uagcaaguuaaaauaaggcuagucc |
| | ucaacuugaaaaaguggcaccgagu | guuaucaacuugaaaaaguggcacc |
| | cggugcUUUU (SEQ ID NO: | gagucggugcU*U*U*U (SEQ |
| | 222) | ID NO: 223) |
| β2M sgRNA spacer | CAGUAAGUCAACUUCAA | C*A*G*UAAGUCAACUU |
| (B2M4 spacer) | UGU (SEQ ID NO: 224) | CAAUGU (SEQ ID NO: |
| | | 225) |
| CD70-T7 (X35365) | GCUUUGGUCCCAUUGGU | G*C*mU*UUGGUCCCAU |
| | CGCguuuuagagcuagaaauagca | UGGUCGCGUUUUAGAG |
| | aguuaaaauaaggcuaguccguuau | CUAGAAAUAGCAAGUU |
| | caacuugaaaaaguggcaccgaguc | AAAAUAAGGCUAGUCC |
| | ggugcuuuu | GUUAUCAACUUGAAAA |
| | (SEQ ID NO: 226) | AGUGGCACCGAGUCGGU |
| | | GCU*U*U*U |
| | | (SEQ ID NO: 227) |
| CD70-T7 (X35365) | GCUUUGGUCCCAUUGGU | G*C*U*UUGGUCCCAUU |
| spacer | CGC (SEQ ID NO: 228) | GGUCGC (SEQ ID NO: 229) |

Target Sequences

| | | Target Sequence-Without |
|---|---|---|
| Guide Name | Target Sequence (PAM) | PAM |
| TRAC TA-1 | AGAGCAACAGTGCTGTG | AGAGCAACAGTGCTGTG |
| | GCC(TGG) (SEQ ID NO: | GCC (SEQ ID NO: 11) |
| | 10) | |
| β2M B2M1 | GCTACTCTCTCTTTCTGG | GCTACTCTCTCTTTCTGG |
| | CC(TGG) (SEQ ID NO: 12) | CC (SEQ ID NO: 13) |
| β2M B2M4 | CAGTAAGTCAACTTCAA | CAGTAAGTCAACTTCAA |
| | TGT(TGG) (SEQ ID NO: 230) | TGT (SEQ ID NO: 231) |
| CD70 T7 (X35365) | GCTTTGGTCCCATTGGTC | GCTTTGGTCCCATTGGTC |
| | GC(GGG) (SEQ ID NO: 232) | GC (SEQ ID NO: 233) |

Exemplary sgRNA Formula
nnnnnnnnnnnnnnnnnnnnguuuuagagcuagaaauagcaaguuaaaauaaggcuaguccguuaucaacuugaaaa
aguggcaccgagucggugcuuuu (SEQ ID NO: 14)
nnnnnnnnnnnnnnnnnnnnguuuuagagcuagaaauagcaaguuaaaauaaggcuaguccguuaucaacuugaaaa
aguggcaccgagucggugc (SEQ ID NO: 15)
n₍₁₇₋₃₀₎guuuuagagcuagaaauagcaaguuaaaauaaggcuaguccguuaucaacuug
aaaaaguggcaccgagucggugcu₍₁₋₈₎ (SEQ ID NO: 16)
*: 2′-O-methyl phosphorothioate residue

TABLE 2
CD83 gRNA Sequences/Target Sequences

| Name | | | |
|---|---|---|---|
| (target | | | Target Sequence |
| strand) | Unmodified Sequence | Modified Sequence | (PAM) |
| CD83_exon2_ | UCCCGCGACGCCGGAGG | U*C*C*CGCGACGCC | TCCCGCGACGCC |
| 1 sgRNA | UGAAGGGUUUUAGAGCU | GGAGGUGAAGGGU | GGAGGTGAAGG |
| (+) | AGAAAUAGCAAGUUAAA | UUUAGAGCUAGAAA | (TGG) |
| | AUAAGGCUAGUCCGUUA | UAGCAAGUUAAAAU | SEQ ID NO: 21) |
| | UCAACUUGAAAAAGUGG | AAGGCUAGUCCGUU | TCCCGCGACGCC |
| | CACCGAGUCGGUGCUUU | AUCAACUUGAAAAA | GGAGGTGAAGG |
| | U (SEQ ID NO: 17) | GUGGCACCGAGUCG | (SEQ ID NO: 22) |
| | | GUGCU*U*U*U (SEQ | |
| | | ID NO: 18) | |
| CD83_exon2_ | UCCCGCGACGCCGGAGG | U*C*C*CGCGACGCC | |
| 1 spacer | UGAAGG (SEQ ID NO: 19) | GGAGGUGAAGG | |
| | | (SEQ ID NO: 20) | |
| CD83_exon2_ | GUAGGGAACCUGCGGAU | G*U*A*GGGAACCUG | GTAGGGAACCTG |
| 3 | CCCAGGGUUUUAGAGCU | CGGAUCCCAGGGUU | CGGATCCCAGG |
| sgRNA (-) | AGAAAUAGCAAGUUAAA | UUAGAGCUAGAAAU | (GGG) (SEQ ID NO: |
| | AUAAGGCUAGUCCGUUA | AGCAAGUUAAAAUA | 27) |
| | UCAACUUGAAAAAGUGG | AGGCUAGUCCGUUA | GTAGGGAACCTG |
| | CACCGAGUCGGUGCUUU | UCAACUUGAAAAAG | CGGATCCCAGG |
| | U (SEQ ID NO: 23) | UGGCACCGAGUCGG | (SEQ ID NO: 28) |
| | | UGCU*U*U*U | |
| | | (SEQ ID NO: 24) | |
| CD83_exon | GUAGGGAACCUGCGGAU | G*U*A*GGGAACCUG | |
| 2_3 sgRNA | CCCAGG (SEQ ID NO: 25) | CGGAUCCCAGG | |
| spacer | | (SEQ ID NO: 26) | |
| CD83_exon | UUGACCCAGGAGACCGU | U*U*G*ACCCAGGAG | TTGACCCAGGAG |
| 2_G1 | GUAGUUUUAGAGCUAGA | ACCGUGUAGUUUUA | ACCGTGTA(GGG) |
| sgRNA (-) | AAUAGCAAGUUAAAAUA | GAGCUAGAAAUAGC | (SEQ ID NO: 71) |
| | AGGCUAGUCCGUUAUCA | AAGUUAAAAUAAG | TTGACCCAGGAG |
| | ACUUGAAAAAGUGGCAC | GCUAGUCCGUUAUC | ACCGTGTA |
| | CGAGUCGGUGCU (SEQ ID | AACUUGAAAAAGUG | (SEQ ID NO: 72) |
| | NO: 99) | GCACCGAGUCGGU* | |
| | | G*C*U (SEQ ID NO: | |
| | | 100) | |
| CD83_exon | UUGACCCAGGAGACCGU | U*U*G*ACCCAGGAG | |
| 2_G1 spacer | GUA (SEQ ID NO: 101) | ACCGUGUA (SEQ ID | |
| | | NO: 102) | |
| CD83_exon | AGGUUCCCUACACGGUC | A*G*G*UUCCCUACA | AGGTTCCCTACA |
| 2_ G2 | UCCGUUUUAGAGCUAGA | CGGUCUCCGUUUUA | CGGTCTCC (TGG) |
| sgRNA (+) | AAUAGCAAGUUAAAAUA | GAGCUAGAAAUAGC | (SEQ ID NO: 73) |
| | AGGCUAGUCCGUUAUCA | AAGUUAAAAUAAG | AGGTTCCCTACA |
| | ACUUGAAAAAGUGGCAC | GCUAGUCCGUUAUC | CGGTCTCC |
| | CGAGUCGGUGCU (SEQ ID | AACUUGAAAAAGUG | (SEQ ID NO: 74) |
| | NO: 103) | GCACCGAGUCGGU* | |
| | | G*C*U (SEQ ID NO: | |
| | | 104) | |
| CD83_exon | AGGUUCCCUACACGGUC | A*G*G*UUCCCUACA | |
| 2_G2 spacer | UCC | CGGUCUCC (SEQ ID | |
| | (SEQ ID NO: 105) | NO: 106) | |
| CD83_exon | GGAUCCGCAGGUUCCCU | G*G*A*UCCGCAGGU | GGATCCGCAGGT |
| 2_G3 | ACAGUUUUAGAGCUAGA | UCCCUACAGUUUUA | TCCCTACA (CGG) |
| sgRNA (+) | AAUAGCAAGUUAAAAUA | GAGCUAGAAAUAGC | (SEQ ID NO: 75) |
| | AGGCUAGUCCGUUAUCA | AAGUUAAAAUAAG | GGATCCGCAGGT |
| | ACUUGAAAAAGUGGCAC | GCUAGUCCGUUAUC | TCCCTACA |
| | CGAGUCGGUGCU (SEQ ID | AACUUGAAAAAGUG | (SEQ ID NO: 76) |
| | NO: 107) | GCACCGAGUCGGU* | |
| | | G*C*U (SEQ ID NO: | |
| | | 108) | |
| CD83_exon | GGAUCCGCAGGUUCCCU | G*G*A*UCCGCAGGU | |
| 2_G3 spacer | ACA | UCCCUACA (SEQ ID | |
| | (SEQ ID NO: 109) | NO: 110) | |
| CD83_exon | GAGACCGUGUAGGGAAC | G*A*G*ACCGUGUAG | GAGACCGTGTAG |
| 2_G4 | CUGGUUUUAGAGCUAGA | GGAACCUGGUUUUA | GGAACCTG (CGG) |
| sgRNA (-) | AAUAGCAAGUUAAAAUA | GAGCUAGAAAUAGC | (SEQ ID NO: 77) |
| | AGGCUAGUCCGUUAUCA | AAGUUAAAAUAAG | GAGACCGTGTAG |
| | ACUUGAAAAAGUGGCAC | GCUAGUCCGUUAUC | GGAACCTG |
| | CGAGUCGGUGCU (SEQ ID | AACUUGAAAAAGUG | (SEQ ID NO: 78) |
| | NO: 111) | GCACCGAGUCGGU* | |
| | | G*C*U (SEQ ID NO: | |
| | | 112) | |
| CD83_exon | GAGACCGUGUAGGGAAC | G*A*G*ACCGUGUAG | |
| 2_G4 spacer | CUG | GGAACCUG (SEQ ID | |
| | (SEQ ID NO: 113) | NO: 114) | |
| CD83_exon | CUUGACCCAGGAGACCG | C*U*U*GACCCAGGA | CTTGACCCAGGA |
| 2_G5 | UGUGUUUUAGAGCUAGA | GACCGUGUGUUUUA | GACCGTGT (AGG) |
| sgRNA (-) | AAUAGCAAGUUAAAAUA | GAGCUAGAAAUAGC | (SEQ ID NO: 79) |
| | AGGCUAGUCCGUUAUCA | AAGUUAAAAUAAG | CTTGACCCAGGA |
| | ACUUGAAAAAGUGGCAC | GCUAGUCCGUUAUC | GACCGTGT |
| | CGAGUCGGUGCU (SEQ ID | AACUUGAAAAAGUG | (SEQ ID NO: 80) |
| | NO: 115) | GCACCGAGUCGGU* | |
| | | G*C*U (SEQ ID NO: | |
| | | 116) | |
| CD83_exon | CUUGACCCAGGAGACCG | C*U*U*GACCCAGGA | |
| 2_G5 spacer | UGU | GACCGUGU (SEQ ID | |
| | (SEQ ID NO: 117) | NO: 118) | |
| CD83_exon | GGUGGCUUGCUCCGAAG | G*G*U*GGCUUGCUC | GGTGGCTTGCTCC |
| 2_G6 | AUGGUUUUAGAGCUAGA | CGAAGAUGGUUUUA | GAAGATG (TGG) |
| sgRNA (+) | AAUAGCAAGUUAAAAUA | GAGCUAGAAAUAGC | (SEQ ID NO: 81) |
| | AGGCUAGUCCGUUAUCA | AAGUUAAAAUAAG | GGTGGCTTGCTCC |
| | ACUUGAAAAAGUGGCAC | GCUAGUCCGUUAUC | GAAGATG |
| | CGAGUCGGUGCU (SEQ ID | AACUUGAAAAAGUG | (SEQ ID NO: 82) |
| | NO: 119) | GCACCGAGUCGGU* | |
| | | G*C*U (SEQ ID NO: | |
| | | 120) | |
| CD83_exon | GGUGGCUUGCUCCGAAG | G*G*U*GGCUUGCUC | |
| 2_G6 spacer | AUG | CGAAGAUG (SEQ ID | |
| | (SEQIDNO: 121) | NO: 122) | |
| CD83_exon | GCACUCUGCAGGACCCG | G*C*A*CUCUGCAGG | GCACTCTGCAGG |
| 3_G7 | GAUGUUUUAGAGCUAGA | ACCCGGAUGUUUUA | ACCCGGAT (GGG) |
| sgRNA (+) | AAUAGCAAGUUAAAAUA | GAGCUAGAAAUAGC | (SEQ ID NO: 83) |
| | AGGCUAGUCCGUUAUCA | AAGUUAAAAUAAG | GCACTCTGCAGG |
| | ACUUGAAAAAGUGGCAC | GCUAGUCCGUUAUC | ACCCGGAT |
| | CGAGUCGGUGCU (SEQ ID | AACUUGAAAAAGUG | (SEQ ID NO: 84) |
| | NO: 123) | GCACCGAGUCGGU* | |
| | | G*C*U (SEQ ID NO: | |
| | | 124) | |
| CD83_exon | GCACUCUGCAGGACCCG | G*C*A*CUCUGCAGG | |
| 3_G7 spacer | GAU (SEQ ID NO: 125) | ACCCGGAU (SEQ ID | |
| | | NO: 126) | |
| CD83_exon | AAACACUACCAGCUGCA | A*A*A*CACUACCAG | AAACACTACCAG |
| 3_G8 | ACUGUUUUAGAGCUAGA | CUGCAACUGUUUUA | CTGCAACT (CGG) |
| sgRNA (+) | AAUAGCAAGUUAAAAUA | GAGCUAGAAAUAGC | (SEQ ID NO: 85) |
| | AGGCUAGUCCGUUAUCA | AAGUUAAAAUAAG | AAACACTACCAG |
| | ACUUGAAAAAGUGGCAC | GCUAGUCCGUUAUC | CTGCAACT |
| | CGAGUCGGUGCU (SEQ ID | AACUUGAAAAAGUG | (SEQ ID NO: 86) |
| | NO: 127) | GCACCGAGUCGGU* | |
| | | G*C*U (SEQ ID NO: | |
| | | 128) | |
| CD83_exon | AAACACUACCAGCUGCA | A*A*A*CACUACCAG | |
| 3_G8 spacer | ACU | CUGCAACU (SEQ ID | |
| | (SEQ ID NO: 129) | NO: 130) | |
| CD83_exon | UAGGGAACCUGCGGAUC | U*A*G*GGAACCUGC | TAGGGAACCTGC |
| 2_G9 | CCAGUUUUAGAGCUAGA | GGAUCCCAGUUUUA | GGATCCCA (GGG) |
| sgRNA (-) | AAUAGCAAGUUAAAAUA | GAGCUAGAAAUAGC | (SEQ ID NO: 87) |
| | AGGCUAGUCCGUUAUCA | AAGUUAAAAUAAG | TAGGGAACCTGC |
| | ACUUGAAAAAGUGGCAC | GCUAGUCCGUUAUC | GGATCCCA |
| | CGAGUCGGUGCU (SEQ ID | AACUUGAAAAAGUG | (SEQ ID NO: 88) |
| | NO: 131) | GCACCGAGUCGGU* | |
| | | G*C*U (SEQ ID NO: | |
| | | 132) | |
| CD83_exon | UAGGGAACCUGCGGAUC | U*A*G*GGAACCUGC | |
| 2_G9 spacer | CCA | GGAUCCCA (SEQ ID | |
| | (SEQ ID NO: 133) | NO: 134) | |
| CD83_exon | UGCACUCUGCAGGACCC | U*G*C*ACUCUGCAG | TGCACTCTGCAG |
| 3_G10 | GGAGUUUUAGAGCUAGA | GACCCGGAGUUUUA | GACCCGGA (TGG) |
| sgRNA (+) | AAUAGCAAGUUAAAAUA | GAGCUAGAAAUAGC | (SEQ ID NO: 89) |
| | AGGCUAGUCCGUUAUCA | AAGUUAAAAUAAG | TGCACTCTGCAG |
| | ACUUGAAAAAGUGGCAC | GCUAGUCCGUUAUC | GACCCGGA |
| | CGAGUCGGUGCU (SEQ ID | AACUUGAAAAAGUG | (SEQ ID NO: 90) |
| | NO: 135) | GCACCGAGUCGGU* | |
| | | G*C*U (SEQ ID NO: | |
| | | 136) | |
| CD83_exon | UGCACUCUGCAGGACCC | U*G*C*ACUCUGCAG | |
| 3_G10 | GGA | GACCCGGA (SEQ ID | |
| spacer | (SEQ ID NO: 137) | NO: 138) | |
| CD83_exon | ACUUGCCCUGCACCGCC | A*C*U*UGCCCUGCA | ACTTGCCCTGCAC |
| 2_G11 | CCCGUUUUAGAGCUAGA | CCGCCCCCGUUUUA | CGCCCCC (TGG) |
| sgRNA (+) | AAUAGCAAGUUAAAAUA | GAGCUAGAAAUAGC | (SEQ ID NO: 91) |
| | AGGCUAGUCCGUUAUCA | AAGUUAAAAUAAG | ACTTGCCCTGCAC |
| | ACUUGAAAAAGUGGCAC | GCUAGUCCGUUAUC | CGCCCCC |
| | CGAGUCGGUGCU (SEQ ID | AACUUGAAAAAGUG | (SEQ ID NO: 92) |
| | NO: 139) | GCACCGAGUCGGU* | |
| | | G*C*U (SEQ ID NO: | |
| | | 140) | |
| CD83_exon | ACUUGCCCUGCACCGCC | A*C*U*UGCCCUGCA | |
| 2_G11 | CCC | CCGCCCCC (SEQ ID | |
| spacer | (SEQ ID NO: 141) | NO: 142) | |
| CD83_exon | AACACUACCAGCUGCAA | A*A*C*ACUACCAGC | AACACTACCAGC |
| 3_G12 | CUCGUUUUAGAGCUAGA | UGCAACUCGUUUUA | TGCAACTC (GGG) |
| sgRNA (+) | AAUAGCAAGUUAAAAUA | GAGCUAGAAAUAGC | (SEQ ID NO: 93) |
| | AGGCUAGUCCGUUAUCA | AAGUUAAAAUAAG | AACACTACCAGC |
| | ACUUGAAAAAGUGGCAC | GCUAGUCCGUUAUC | TGCAACTC |
| | CGAGUCGGUGCU (SEQ ID | AACUUGAAAAAGUG | (SEQ ID NO: 94) |
| | NO: 143) | GCACCGAGUCGGU* | |
| | | G*C*U (SEQ ID NO: | |
| | | 144) | |
| CD83_exon | AACACUACCAGCUGCAA | A*A*C*ACUACCAGC | |
| 3_G12 | CUC | UGCAACUC (SEQ ID | |
| spacer | (SEQ ID NO: 145) | NO: 146) | |
| CD83_exon | CACCGCCCCCUGGGAUC | C*A*C*CGCCCCCUG | CACCGCCCCCTG |
| 2_G13 | CGCGUUUUAGAGCUAGA | GGAUCCGCGUUUUA | GGATCCGC (AGG) |
| sgRNA (+) | AAUAGCAAGUUAAAAUA | GAGCUAGAAAUAGC | (SEQ ID NO: 95) |
| | AGGCUAGUCCGUUAUCA | AAGUUAAAAUAAG | CACCGCCCCCTG |
| | ACUUGAAAAAGUGGCAC | GCUAGUCCGUUAUC | GGATCCGC |
| | CGAGUCGGUGCU (SEQ ID | AACUUGAAAAAGUG | (SEQ ID NO: 96) |
| | NO: 147) | GCACCGAGUCGGU* | |
| | | G*C*U (SEQ ID NO: | |
| | | 148) | |
| CD83_exon | CACCGCCCCCUGGGAUC | C*A*C*CGCCCCCUG | |
| 2_G13 | CGC | GGAUCCGC (SEQ ID | |
| spacer | (SEQ ID NO: 149) | NO: 150) | |
| CD83_exon | GGAUCCCAGGGGGCGGU | G*G*A*UCCCAGGGG | GGATCCCAGGGG |
| 2_G14 | GCAGUUUUAGAGCUAGA | GCGGUGCAGUUUUA | GCGGTGCA (GGG) |
| sgRNA (-) | AAUAGCAAGUUAAAAUA | GAGCUAGAAAUAGC | (SEQ ID NO: 97) |
| | AGGCUAGUCCGUUAUCA | AAGUUAAAAUAAG | GGATCCCAGGGG |
| | ACUUGAAAAAGUGGCAC | GCUAGUCCGUUAUC | GCGGTGCA |
| | CGAGUCGGUGCU (SEQ ID | AACUUGAAAAAGUG | (SEQ ID NO: 98) |
| | NO: 151) | GCACCGAGUCGGU* | |
| | | G*C*U (SEQ ID NO: | |
| | | 152) | |
| CD83_exon | GGAUCCCAGGGGGCGGU | G*G*A*UCCCAGGGG | |
| 2_G14 | GCA | GCGGUGCA (SEQ ID | |
| spacer | (SEQ ID NO: 153) | NO: 154) | |
| CD83_exon | UCCCGCGACGCCGGAGG | U*C*C*CGCGACGCC | TCCCGCGACGCC |
| 2_G15 | UGAGUUUUAGAGCUAGA | GGAGGUGAGUUUU | GGAGGTGA |
| sgRNA (+) | AAUAGCAAGUUAAAAUA | AGAGCUAGAAAUAG | (AGG) |
| | AGGCUAGUCCGUUAUCA | CAAGUUAAAAUAAG | (SEQ ID NO: 160) |
| | ACUUGAAAAAGUGGCAC | GCUAGUCCGUUAUC | TCCCGCGACGCC |
| | CGAGUCGGUGCU (SEQ ID | AACUUGAAAAAGUG | GGAGGTGA |
| | NO: 156) | GCACCGAGUCGGUG | (SEQ ID NO: 161) |
| | | *C*U* (SEQ ID NO: | |
| | | 157) | |
| CD83_exon | UCCCGCGACGCCGGAGG | U*C*C*CGCGACGCC | |
| 2_G15 | UGA | GGAGGUGA (SEQ ID | |
| spacer | (SEQ ID NO: 158) | NO: 159) | |
| CD83_exon | GUAGGGAACCUGCGGAU | G*U*A*GGGAACCUG | GTAGGGAACCTG |
| 2_G16 | CCCGUUUUAGAGCUAGA | CGGAUCCCGUUUUA | CGGATCCC (AGG) |
| sgRNA (-) | AAUAGCAAGUUAAAAUA | GAGCUAGAAAUAGC | (SEQ ID NO: 166) |
| | AGGCUAGUCCGUUAUCA | AAGUUAAAAUAAG | GTAGGGAACCTG |
| | ACUUGAAAAAGUGGCAC | GCUAGUCCGUUAUC | CGGATCCC |
| | CGAGUCGGUGCU (SEQ ID | AACUUGAAAAAGUG | (SEQ ID NO: 167) |
| | NO: 162) | GCACCGAGUCGGUG | |
| | | *C*U* (SEQ ID NO: | |
| | | 163) | |
| CD83_exon | GUAGGGAACCUGCGGAU | G*U*A*GGGAACCUG | |
| 2_G16 | CCC (SEQ ID NO: 164) | CGGAUCCC (SEQ ID | |
| spacer | | NO: 165) | |
| CD83-17 | AAGCUGGAGGCCGCGCG | A*A*G*CUGGAGGCC | AAGCTGGAGGCC |
| sgRNA | ACAGUUUUAGAGCUAGA | GCGCGACAGUUUUA | AAGCTGGAGGCC |
| | AAUAGCAAGUUAAAAUA | GAGCUAGAAAUAGC | GCGCGACA (TGG) |
| | AGGCUAGUCCGUUAUCA | AAGUUAAAAUAAG | (SEQ ID NO: 168) |
| | ACUUGAAAAAGUGGCAC | GCUAGUCCGUUAUC | GCGCGACA |
| | CGAGUCGGUGCU | AACUUGAAAAAGUG | (SEQ ID NO: 169) |
| | (SEQ ID NO: 174) | GCACCGAGUCGGU* | |
| | | G*C*U | |
| | | (SEQ ID NO: 175) | |
| CD83-17 | AAGCUGGAGGCCGCGCG | A*A*G*CUGGAGGCC | |
| spacer | ACA | GCGCGACA (SEQ ID | |
| | (SEQ ID NO: 176) | NO: 177) | |
| CD83-18 | GCUGCAACUCGGGGACA | G*C*U*GCAACUCGG | GCTGCAACTCGG |
| sgRNA | UACGUUUUAGAGCUAGA | GGACAUACGUUUUA | GGACATAC (AGG) |
| | AAUAGCAAGUUAAAAUA | GAGCUAGAAAUAGC | (SEQ ID NO: 170) |
| | AGGCUAGUCCGUUAUCA | AAGUUAAAAUAAG | GCTGCAACTCGG |
| | ACUUGAAAAAGUGGCAC | GCUAGUCCGUUAUC | GGACATAC |
| | CGAGUCGGUGCU (SEQ ID | AACUUGAAAAAGUG | (SEQ ID NO: 171) |
| | NO: 178) | GCACCGAGUCGGU* | |
| | | G*C*U (SEQ ID NO: | |
| | | 179) | |
| CD83-18 | GCUGCAACUCGGGGACA | G*C*U*GCAACUCGG | |
| spacer | UAC (SEQ ID NO: 180) | GGACAUAC (SEQ ID | |
| | | NO: 181) | |
| CD83-21 | GGUUCCCUACACGGUCU | G*G*U*UCCCUACAC | GGTTCCCTACAC |
| sgRNA | CCUGUUUUAGAGCUAGA | GGUCUCCUGUUUUA | GGTCTCCT (GGG) |
| | AAUAGCAAGUUAAAAUA | GAGCUAGAAAUAGC | (SEQ ID NO: 172) |
| | AGGCUAGUCCGUUAUCA | AAGUUAAAAUAAG | GGTTCCCTACAC |
| | ACUUGAAAAAGUGGCAC | GCUAGUCCGUUAUC | GGTCTCCT |
| | CGAGUCGGUGCU (SEQ ID | AACUUGAAAAAGUG | (SEQ ID NO: 173) |
| | NO: 182) | GCACCGAGUCGGU* | |
| | | G*C*U (SEQ ID NO: | |
| | | 183) | |
| CD83-21 | GGUUCCCUACACGGUCU | G*G*U*UCCCUACAC | |
| spacer | CCU (SEQ ID NO: 184) | GGUCUCCU (SEQ ID | |
| | | NO: 185) | |
| CD83-19 | CGCGAGCCCUACCGCAG | C*G*C*GAGCCCUAC | CGCGAGCCCTAC |
| sgRNA | CUCGUUUUAGAGCUAGA | CGCAGCUCGUUUUA | CGCAGCTC (AGG) |
| | AAUAGCAAGUUAAAAUA | GAGCUAGAAAUAGC | SEQ ID NO: 186) |
| | AGGCUAGUCCGUUAUCA | AAGUUAAAAUAAG | CGCGAGCCCTAC |
| | ACUUGAAAAAGUGGCAC | GCUAGUCCGUUAUC | CGCAGCTC |
| | CGAGUCGGUGCU | AACUUGAAAAAGUG | (SEQ ID NO: 187) |
| | SEQ ID NO: 198) | GCACCGAGUCGGU* | |
| | | G*C*U SEQ ID NO: | |
| | | 199) | |
| CD83-19 | CGCGAGCCCUACCGCAG | C*G*C*GAGCCCUAC | |
| spacer | CUC | CGCAGCUC (SEQ ID | |
| | SEQ ID NO: 200) | NO: 201) | |
| CD83-20 | GUAUGUCCCCGAGUUGC | G*U*A*UGUCCCCGA | GTATGTCCCCGA |
| sgRNA | AGCGUUUUAGAGCUAGA | GUUGCAGCGUUUUA | GTTGCAGC (TGG) |
| | AAUAGCAAGUUAAAAUA | GAGCUAGAAAUAGC | (SEQ ID NO: 188) |
| | AGGCUAGUCCGUUAUCA | AAGUUAAAAUAAG | GTATGTCCCCGA |
| | ACUUGAAAAAGUGGCAC | GCUAGUCCGUUAUC | GTTGCAGC |
| | CGAGUCGGUGCU | AACUUGAAAAAGUG | (SEQ ID NO: 189) |
| | (SEQ ID NO: 202) | GCACCGAGUCGGU* | |
| | | G*C*U (SEQ ID NO: | |
| | | 203) | |
| CD83-20 | GUAUGUCCCCGAGUUGC | G*U*A*UGUCCCCGA | |
| spacer | AGC (SEQ ID NO: 204) | GUUGCAGC (SEQ ID | |
| | | NO: 205) | |
| CD83-22 | CCUCCGGCGUCGCGGGA | C*C*U*CCGGCGUCG | CCTCCGGCGTCG |
| sgRNA | GCCGUUUUAGAGCUAGA | CGGGAGCCGUUUUA | CGGGAGCC (AGG) |
| | AAUAGCAAGUUAAAAUA | GAGCUAGAAAUAGC | (SEQ ID NO: 190) |
| | AGGCUAGUCCGUUAUCA | AAGUUAAAAUAAG | CCTCCGGCGTCG |
| | ACUUGAAAAAGUGGCAC | GCUAGUCCGUUAUC | CGGGAGCC |
| | CGAGUCGGUGCU (SEQ ID | AACUUGAAAAAGUG | (SEQ ID NO: 191) |
| | NO: 206) | GCACCGAGUCGGU* | |
| | | G*C*U (SEQ ID NO: | |
| | | 207) | |
| CD83-22 | CCUCCGGCGUCGCGGGA | C*C*U*CCGGCGUCG | |
| spacer | GCC (SEQ ID NO: 208) | CGGGAGCC (SEQ ID | |
| | | NO: 209) | |
| CD83-23 | UCUGCUCCUGAGCUGCG | U*C*U*GCUCCUGAG | TCTGCTCCTGAGC |
| sgRNA | GUAGUUUUAGAGCUAGA | CUGCGGUAGUUUUA | TGCGGTA (GGG) |
| | AAUAGCAAGUUAAAAUA | GAGCUAGAAAUAGC | (SEQ ID NO: 192) |
| | AGGCUAGUCCGUUAUCA | AAGUUAAAAUAAG | TCTGCTCCTGAGC |
| | ACUUGAAAAAGUGGCAC | GCUAGUCCGUUAUC | TGCGGTA |
| | CGAGUCGGUGCU (SEQ ID | AACUUGAAAAAGUG | (SEQ ID NO: 193) |
| | NO: 210) | GCACCGAGUCGGU* | |
| | | G*C*U (SEQ ID NO: | |
| | | 211) | |
| CD83-23 | UCUGCUCCUGAGCUGCG | U*C*U*GCUCCUGAG | |
| spacer | GUA (SEQ ID NO: 212) | CUGCGGUA (SEQ ID | |
| | | NO: 213) | |
| CD83-24 | CGUCGCGGGAGCCAGGC | C*G*U*CGCGGGAGC | CGTCGCGGGAGC |
| sgRNA | UGUGUUUUAGAGCUAGA | CAGGCUGUGUUUUA | CAGGCTGT (AGG) |
| | AAUAGCAAGUUAAAAUA | GAGCUAGAAAUAGC | (SEQ ID NO: 194) |
| | AGGCUAGUCCGUUAUCA | AAGUUAAAAUAAG | CGTCGCGGGAGC |
| | ACUUGAAAAAGUGGCAC | GCUAGUCCGUUAUC | CAGGCTGT |
| | CGAGUCGGUGCU (SEQ ID | AACUUGAAAAAGUG | (SEQ ID NO: 195) |
| | NO: 214) | GCACCGAGUCGGU* | |
| | | G*C*U (SEQ ID NO: | |
| | | 215) | |
| CD83-24 | CGUCGCGGGAGCCAGGC | C*G*U*CGCGGGAGC | |
| spacer | UGU (SEQ ID NO: 216) | CAGGCUGU (SEQ ID | |
| | | NO: 217) | |
| CD83-25 | AGCGGAGAUUGUCCUGC | A*G*C*GGAGAUUG | AGCGGAGATTGT |
| sgRNA | UGCGUUUUAGAGCUAGA | UCCUGCUGCGUUUU | CCTGCTGC (TGG) |
| | AAUAGCAAGUUAAAAUA | AGAGCUAGAAAUAG | (SEQ ID NO: 196) |
| | AGGCUAGUCCGUUAUCA | CAAGUUAAAAUAAG | AGCGGAGATTGT |
| | ACUUGAAAAAGUGGCAC | GCUAGUCCGUUAUC | CCTGCTGC |
| | CGAGUCGGUGCU (SEQ ID | AACUUGAAAAAGUG | (SEQ ID NO: 197) |
| | NO: 218) | GCACCGAGUCGGU* | |
| | | G*C*U (SEQ ID NO: | |
| | | 219) | |
| CD83-25 | AGCGGAGAUUGUCCUGC | A*G*C*GGAGAUUG | |
| spacer | UGC (SEQ ID NO: 220) | UCCUGCUGC (SEQ ID | |
| | | NO: 221) | |

| | |
|---|---|
| Scaffold | GUUUUAGAGCUAGAAAUAGCAAGUUAAAAUAAGGCUAGUCCGUUA |
| Sequence 1 | UCAACUUGAAAAAGUGGCACCGAGUCGGU*G*C*U (SEQ ID NO: 155) |
| Scaffold | GUUUUAGAGCUAGAAAUAGCAAGUUAAAAUAAGGCUAGUCCGUUA |
| Sequence 2 | UCAACUUGAAAAAGUGGCACCGAGUCGGUGCU*U*U*U (SEQ ID NO: 234) |

*: 2′-O-methyl phosphorothioate residue

TABLE 3
Exemplary Nucleotide Sequences in Disrupted TRAC Gene

| | Sequence (Deletions indicated by dashes | |
|---|---|---|
| Description | (-); insertions indicated by bold) | SEQ ID NO: |
| TRAC gene | AA--------------------- | 29 |
| edit | GAGCAACAAATCTGACT | |
| TRAC gene | AAGAGCAACAGTGCTGT- | 30 |
| edit | GCCTGGAGCAACAAATCTGACT | |
| TRAC gene | AAGAGCAACAGTG------- | 31 |
| edit | CTGGAGCAACAAATCTGACT | |
| TRAC gene | AAGAGCAACAGT------ | 32 |
| edit | GCCTGGAGCAACAAATCTGACT | |
| TRAC gene | AAGAGCAACAGTG--------------------- | 33 |
| edit | CTGACT | |
| TRAC gene | AAGAGCAACAGTGCTGTGGGCCTGGAGCAACAAATCT | 34 |
| edit | GACT | |
| TRAC gene | AAGAGCAACAGTGC | 35 |
| edit | TGGCCTGGAGCAACAAATCTGACT | |
| TRAC gene | AAGAGCAACAGTGCTGTGTGCCTGGAGCAACAAATCT | 36 |
| edit | GACT | |

TABLE 4
Exemplary Nucleotide Sequences in Disrupted β2M Gene

| | Sequence (Deletions indicated by dashes (-); | SEQ ID |
|---|---|---|
| Description | insertions indicated by bold) | NO: |
| β2M gene-edit | CGTGGCCTTAGCTGTGCTCGCGCTACTCTCTCTTTCT- | 37 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 38 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 39 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAG | 40 |
| | CCTGGAGGCTATCCAGCGTGAGTCTCTCCTACCCTCCCGCT | |
| β2M gene-edit | CGTGGCCTTAGCTGTGCTCGC---------------------- | 41 |
| | ---GCTATCCAGCGTGAGTCTCTCCTACCCTCCCGCT | |
| β2M gene-edit | CGTGGCCTTAGCTGTGCTCGCGCTACTCTCTCTTTCTGTGGCC | 42 |
| | TGGAGGCTATCCAGCGTGAGTCTCTCCTACCCTCCCGCT | |

TABLE 5
Exemplary Sequences of Anti-CD83 CAR Components

| | | SEQ ID |
|---|---|---|
| Name | Sequence | NO: |
| anti-CD83 | TGGYWWT | 43 |
| antibody | | |
| VH | | |
| CDR1 | | |
| anti-CD83 | YIFSSGNTNYNPSIKS | 44 |
| antibody | | |
| VH | | |
| CDR2 | | |
| anti-CD83 | AYGKLGFDY | 45 |
| antibody | | |
| VH | | |
| CDR3 | | |
| anti-CD83 | TLSSQHSTYTIG | 46 |
| antibody | | |
| VL CDR1 | | |
| anti-CD83 | VNSDGSHSKGD | 47 |
| antibody | | |
| VL CDR2 | | |
| anti-CD83 | GSSDSSGYV | 48 |
| antibody | | |
| VL CDR3 | | |
| Anti- | QVQLKESGPGLVKPSQSLSLTCSVTGFSITTGGYWWTWIRQFPGQKLEWMGYI | 49 |
| CD83 VH | FSSGNTNYNPSIKSRISITRDTSKNQFFLQLNSVTTEGDTARYYCARAYGKLG | |
| | FDYWGQGTLVTVS | |
| Anti- | QPVLTQSPSASASLGNSVKITCTLSSQHSTYTIGWYQQHPDKAPKYVMYVNSD | 50 |
| CD83 VL | GSHSKGDGIPDRFSGSSSGAHRYLSISNIQPEDEADYFCGSSDSSGYVFGSGT | |
| | QLTVL | |
| Anti- | QVQLKESGPGLVKPSQSLSLTCSVTGFSITTGGYWWTWIRQFPGQKLEWMGYI | 51 |
| CD83 | FSSGNTNYNPSIKSRISITRDTSKNQFFLQLNSVTTEGDTARYYCARAYGKLG | |
| scFv | FDYWGQGTLVTVSSGGGGSGGGGSGGGGSQPVLTQSPSASASLGNSVKITCTL | |
| | SSQHSTYTIGWYQQHPDKAPKYVMYVNSDGSHSKGDGIPDRFSGSSSGAHRYL | |
| | SISNIQPEDEADYFCGSSDSSGYVFGSGTQLTVL | |
| Anti- | MALPVTALLLPLALLLHAARPQVQLKESGPGLVKPSQSLSLTCSVTGFSITTG | 52 |
| CD83 | GYWWTWIRQFPGQKLEWMGYIFSSGNTNYNPSIKSRISITRDTSKNQFFLQLN | 235 |
| CAR | SVTTEGDTARYYCARAYGKLGFDYWGQGTLVTVSSGGGGSGGGGSGGGGSQPV | (without |
| | LTQSPSASASLGNSVKITCTLSSQHSTYTIGWYQQHPDKAPKYVMYVNSDGSH | signal |
| | SKGDGIPDRFSGSSSGAHRYLSISNIQPEDEADYFCGSSDSSGYVFGSGTQLT | peptide) |
| | VLRAAATTTPAPRPPTPAPTIASQPLSLRPEACRPAAGGAVHTRGLDFACDIY | |
| | IWAPLAGTCGVLLLSLVITLYCKRGRKKLLYIFKQPFMRPVQTTQEEDGCSCR | |
| | FPEEEEGGCELRVKFSRSADAPAYKQGQNQLYNELNLGRREEYDVLDKRRGRD | |
| | PEMGGKPRRKNPQEGLYNELQKDKMAEAYSEIGMKGERRRGKGHDGLYQGLST | |
| | ATKDTYDALHMQALPPR | |
| Signal | MALPVTALLLPLALLLHAARP | 53 |
| Peptide 1 | | |
| Signal | MLLLVTSLLLCELPHPAFLLIP | 54 |
| Peptide 2 | | |
| 4-1BB | KRGRKKLLYIFKQPFMRPVQTTQEEDGCSCRFPEEEEGGCEL | 55 |
| CoS | | |
| CD28 | SKRSRLLHSDYMNMTPRRPGPTRKHYQPYAPPRDFAAYRS | 56 |
| CoS | | |
| CD3ζ | RVKFSRSADAPAYQQGQNQLYNELNLGRREEYDVLDKRRGRDPEMGGKPRRKN | 57 |
| signaling | PQEGLYNELQKDKMAEAYSEIGMKGERRRGKGHDGLYQGLSTATKDTYDALHM | |
| | QALPPR | |
| CD8a | TTTPAPRPPTPAPTIASQPLSLRPEACRPAAGGAVHTRGLDFACDIYIWAPLA | 58 |
| TM/Hinge | GTCGVLLLSLVITLYC | |

TABLE 6
Exemplary AAV Donor Template Sequences

| | | SEQ ID |
|---|---|---|
| Name | Sequence | NO: |
| Left ITR | TTGGCCACTCCCTCTCTGCGCGCTCGCTCGCTCACTGAGGCCGGGCGACCAAA | 59 |
| (5’ ITR) | GGTCGCCCGACGCCCGGGCTTTGCCCGGGCGGCCTCAGTGAGCGAGCGAGCGC | |
| | GCAGAGAGGGAGTGGCCAACTCCATCACTAGGGGTTCCT | |
| Left ITR | CCTGCAGGCAGCTGCGCGCTCGCTCGCTCACTGAGGCCGCCCGGGCGTCGGGC | 60 |
| (5’ ITR) | GACCTTTGGTCGCCCGGCCTCAGTGAGCGAGCGAGCGCGCAGAGAGGGAGTGG | |
| (alternate) | CCAACTCCATCACTAGGGGTTCCT | |
| Right ITR | AGGAACCCCTAGTGATGGAGTTGGCCACTCCCTCTCTGCGCGCTCGCTCGCTC | 61 |
| (3’ ITR) | ACTGAGGCCGCCCGGGCAAAGCCCGGGCGTCGGGCGACCTTTGGTCGCCCGGC | |
| | CTCAGTGAGCGAGCGAGCGCGCAGAGAGGGAGTGGCCAA | |
| Right ITR | AGGAACCCCTAGTGATGGAGTTGGCCACTCCCTCTCTGCGCGCTCGCTCGCTC | 62 |
| (3’ ITR) | ACTGAGGCCGGGCGACCAAAGGTCGCCCGACGCCCGGGCTTTGCCCGGGCGGC | |
| (alternate) | CTCAGTGAGCGAGCGAGCGCGCAGCTGCCTGCAGG | |
| TRAC- | GAGATGTAAGGAGCTGCTGTGACTTGCTCAAGGCCTTATATCGAGTAAACGGT | 63 |
| LHA | AGTGCTGGGGCTTAGACGCAGGTGTTCTGATTTATAGTTCAAAACCTCTATCA | |
| (800 bp) | ATGAGAGAGCAATCTCCTGGTAATGTGATAGATTTCCCAACTTAATGCCAACA | |
| | TACCATAAACCTCCCATTCTGCTAATGCCCAGCCTAAGTTGGGGAGACCACTC | |
| | CAGATTCCAAGATGTACAGTTTGCTTTGCTGGGCCTTTTTCCCATGCCTGCCT | |
| | TTACTCTGCCAGAGTTATATTGCTGGGGTTTTGAAGAAGATCCTATTAAATAA | |
| | AAGAATAAGCAGTATTATTAAGTAGCCCTGCATTTCAGGTTTCCTTGAGTGGC | |
| | AGGCCAGGCCTGGCCGTGAACGTTCACTGAAATCATGGCCTCTTGGCCAAGAT | |
| | TGATAGCTTGTGCCTGTCCCTGAGTCCCAGTCCATCACGAGCAGCTGGTTTCT | |
| | AAGATGCTATTTCCCGTATAAAGCATGAGACCGTGACTTGCCAGCCCCACAGA | |
| | GCCCCGCCCTTGTCCATCACTGGCATCTGGACTCCAGCCTGGGTTGGGGCAAA | |
| | GAGGGAAATGAGATCATGTCCTAACCCTGATCCTCTTGTCCCACAGATATCCA | |
| | GAACCCTGACCCTGCCGTGTACCAGCTGAGAGACTCTAAATCCAGTGACAAGT | |
| | CTGTCTGCCTATTCACCGATTTTGATTCTCAAACAAATGTGTCACAAAGTAAG | |
| | GATTCTGATGTGTATATCACAGACAAAACTGTGCTAGACATGAGGTCTATGGA | |
| | CTTCA | |
| TRAC- | TGGAGCAACAAATCTGACTTTGCATGTGCAAACGCCTTCAACAACAGCATTAT | 64 |
| RHA | TCCAGAAGACACCTTCTTCCCCAGCCCAGGTAAGGGCAGCTTTGGTGCCTTCG | |
| (800 bp) | CAGGCTGTTTCCTTGCTTCAGGAATGGCCAGGTTCTGCCCAGAGCTCTGGTCA | |
| | ATGATGTCTAAAACTCCTCTGATTGGTGGTCTCGGCCTTATCCATTGCCACCA | |
| | AAACCCTCTTTTTACTAAGAAACAGTGAGCCTTGTTCTGGCAGTCCAGAGAAT | |
| | GACACGGGAAAAAAGCAGATGAAGAGAAGGTGGCAGGAGAGGGCACGTGGCCC | |
| | AGCCTCAGTCTCTCCAACTGAGTTCCTGCCTGCCTGCCTTTGCTCAGACTGTT | |
| | TGCCCCTTACTGCTCTTCTAGGCCTCATTCTAAGCCCCTTCTCCAAGTTGCCT | |
| | CTCCTTATTTCTCCCTGTCTGCCAAAAAATCTTTCCCAGCTCACTAAGTCAGT | |
| | CTCACGCAGTCACTCATTAACCCACCAATCACTGATTGTGCCGGCACATGAAT | |
| | GCACCAGGTGTTGAAGTGGAGGAATTAAAAAGTCAGATGAGGGGTGTGCCCAG | |
| | AGGAAGCACCATTCTAGTTGGGGGAGCCCATCTGTCAGCTGGGAAAAGTCCAA | |
| | ATAACTTCAGATTGGAATGTGTTTTAACTCAGGGTTGAGAAAACAGCTACCTT | |
| | CAGGACAAAAGTCAGGGAAGGGCTCTCTGAAGAAATGCTACTTGAAGATACCA | |
| | GCCCTACCAAGGGCAGGGAGAGGACCCTATAGAGGCCTGGGACAGGAGCTCAA | |
| | TGAGAAAGG | |
| EF1a | GGCTCCGGTGCCCGTCAGTGGGCAGAGCGCACATCGCCCACAGTCCCCGAGAA | 65 |
| | GTTGGGGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGTGGG | |
| | GGAGAACCGTATATAAGTGCAGTAGTCGCCGTGAACGTTCTTTTTCGCAACGG | |
| | GTTTGCCGCCAGAACACAGGTAAGTGCCGTGTGTGGTTCCCGCGGGCCTGGCC | |
| | TCTTTACGGGTTATGGCCCTTGCGTGCCTTGAATTACTTCCACTGGCTGCAGT | |
| | ACGTGATTCTTGATCCCGAGCTTCGGGTTGGAAGTGGGTGGGAGAGTTCGAGG | |
| | CCTTGCGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTCTCGCTG | |
| | CTTTCGATAAGTCTCTAGCCATTTAAAATTTTTGATGACCTGCTGCGACGCTT | |
| | TTTTTCTGGCAAGATAGTCTTGTAAATGCGGGCCAAGATCTGCACACTGGTAT | |
| | TTCGGTTTTTGGGGCCGCGGGCGGCGACGGGGCCCGTGCGTCCCAGCGCACAT | |
| | GTTCGGCGAGGCGGGGCCTGCGAGCGCGGCCACCGAGAATCGGACGGGGGTAG | |
| | TCTCAAGCTGGCCGGCCTGCTCTGGTGCCTGGCCTCGCGCCGCCGTGTATCGC | |
| | CCCGCCCTGGGCGGCAAGGCTGGCCCGGTCGGCACCAGTTGCGTGAGCGGAAA | |
| | GATGGCCGCTTCCCGGCCCTGCTGCAGGGAGCTCAAAATGGAGGACGCGGCGC | |
| | TCGGGAGAGCGGGCGGGTGAGTCACCCACACAAAGGAAAAGGGCCTTTCCGTC | |
| | CTCAGCCGTCGCTTCATGTGACTCCACGGAGTACCGGGCGCCGTCCAGGCACC | |
| | TCGATTAGTTCTCGAGCTTTTGGAGTACGTCGTCTTTAGGTTGGGGGGAGGGG | |
| | TTTTATGCGATGGAGTTTCCCCACACTGAGTGGGTGGAGACTGAAGTTAGGCC | |
| | AGCTTGGCACTTGATGTAATTCTCCTTGGAATTTGCCCTTTTTGAGTTTGGAT | |
| | CTTGGTTCATTCTCAAGCCTCAGACAGTGGTTCAAAGTTTTTTTCTTCCATTT | |
| | CAGGTGTCGTGA | |
| LHA-CD83 | GAGATGTAAGGAGCTGCTGTGACTTGCTCAAGGCCTTATATCGAGTAAACGGT | 66 |
| CAR-RHA | AAGAATAAGCAGTATTATTAAGTAGCCCTGCATTTCAGGTTTCCTTGAGTGGC | |
| | AGGCCAGGCCTGGCCGTGAACGTTCACTGAAATCATGGCCTCTTGGCCAAGAT | |
| | TGATAGCTTGTGCCTGTCCCTGAGTCCCAGTCCATCACGAGCAGCTGGTTTCT | |
| | AAGATGCTATTTCCCGTATAAAGCATGAGACCGTGACTTGCCAGCCCCACAGA | |
| | GCCCCGCCCTTGTCCATCACTGGCATCTGGACTCCAGCCTGGGTTGGGGCAAA | |
| | GAGGGAAATGAGATCATGTCCTAACCCTGATCCTCTTGTCCCACAGATATCCA | |
| | GAACCCTGACCCTGCCGTGTACCAGCTGAGAGACTCTAAATCCAGTGACAAGT | |
| | CTGTCTGCCTATTCACCGATTTTGATTCTCAAACAAATGTGTCACAAAGTAAG | |
| | GATTCTGATGTGTATATCACAGACAAAACTGTGCTAGACATGAGGTCTATGGA | |
| | CTTCAGGCTCCGGTGCCCGTCAGTGGGCAGAGCGCACATCGCCCACAGTCCCC | |
| | GAGAAGTTGGGGGGAGGGGTCGGCAATTGAACCGGTGCCTAGAGAAGGTGGCG | |
| | CGGGGTAAACTGGGAAAGTGATGTCGTGTACTGGCTCCGCCTTTTTCCCGAGG | |
| | GTGGGGGAGAACCGTATATAAGTGCAGTAGTCGCCGTGAACGTTCTTTTTCGC | |
| | AACGGGTTTGCCGCCAGAACACAGGTAAGTGCCGTGTGTGGTTCCCGCGGGCC | |
| | TGGCCTCTTTACGGGTTATGGCCCTTGCGTGCCTTGAATTACTTCCACTGGCT | |
| | GCAGTACGTGATTCTTGATCCCGAGCTTCGGGTTGGAAGTGGGTGGGAGAGTT | |
| | CGAGGCCTTGCGCTTAAGGAGCCCCTTCGCCTCGTGCTTGAGTTGAGGCCTGG | |
| | CCTGGGCGCTGGGGCCGCCGCGTGCGAATCTGGTGGCACCTTCGCGCCTGTCT | |
| | AGTGCTGGGGCTTAGACGCAGGTGTTCTGATTTATAGTTCAAAACCTCTATCA | |
| | ATGAGAGAGCAATCTCCTGGTAATGTGATAGATTTCCCAACTTAATGCCAACA | |
| | TACCATAAACCTCCCATTCTGCTAATGCCCAGCCTAAGTTGGGGAGACCACTC | |
| | CAGATTCCAAGATGTACAGTTTGCTTTGCTGGGCCTTTTTCCCATGCCTGCCT | |
| | TTACTCTGCCAGAGTTATATTGCTGGGGTTTTGAAGAAGATCCTATTAAATAA | |
| | CGCTGCTTTCGATAAGTCTCTAGCCATTTAAAATTTTTGATGACCTGCTGCGA | |
| | CGCTTTTTTTCTGGCAAGATAGTCTTGTAAATGCGGGCCAAGATCTGCACACT | |
| | GGTATTTCGGTTTTTGGGGCCGCGGGCGGCGACGGGGCCCGTGCGTCCCAGCG | |
| | CACATGTTCGGCGAGGCGGGGCCTGCGAGCGCGGCCACCGAGAATCGGACGGG | |
| | GGTAGTCTCAAGCTGGCCGGCCTGCTCTGGTGCCTGGCCTCGCGCCGCCGTGT | |
| | ATCGCCCCGCCCTGGGCGGCAAGGCTGGCCCGGTCGGCACCAGTTGCGTGAGC | |
| | GGAAAGATGGCCGCTTCCCGGCCCTGCTGCAGGGAGCTCAAAATGGAGGACGC | |
| | GGCGCTCGGGAGAGCGGGCGGGTGAGTCACCCACACAAAGGAAAAGGGCCTTT | |
| | CCGTCCTCAGCCGTCGCTTCATGTGACTCCACGGAGTACCGGGCGCCGTCCAG | |
| | GCACCTCGATTAGTTCTCGAGCTTTTGGAGTACGTCGTCTTTAGGTTGGGGGG | |
| | AGGGGTTTTATGCGATGGAGTTTCCCCACACTGAGTGGGTGGAGACTGAAGTT | |
| | AGGCCAGCTTGGCACTTGATGTAATTCTCCTTGGAATTTGCCCTTTTTGAGTT | |
| | TGGATCTTGGTTCATTCTCAAGCCTCAGACAGTGGTTCAAAGTTTTTTTCTTC | |
| | CATTTCAGGTGTCGTGACCACCATGGCGCTTCCGGTGACAGCACTGCTCCTCC | |
| | CCTTGGCGCTGTTGCTCCACGCAGCAAGGCCGCAGGTTCAGCTGAAAGAATCT | |
| | GGCCCCGGCCTGGTTAAACCGAGTCAGAGTTTGAGCTTGACTTGTTCTGTGAC | |
| | TGGATTCTCAATAACAACCGGTGGATACTGGTGGACCTGGATACGACAATTTC | |
| | CGGGCCAAAAGCTCGAGTGGATGGGCTATATCTTTAGTAGCGGCAACACTAAC | |
| | TATAATCCTTCAATCAAGAGCAGGATAAGTATCACCCGAGACACCAGTAAGAA | |
| | CCAATTCTTTTTGCAGCTTAACAGCGTTACGACTGAAGGTGACACCGCAAGGT | |
| | ATTACTGCGCGAGGGCCTACGGAAAACTTGGTTTTGATTATTGGGGTCAAGGG | |
| | ACACTGGTAACGGTTTCTAGTGGGGGAGGTGGTAGTGGCGGTGGAGGTTCCGG | |
| | GGGTGGAGGATCCCAACCCGTCCTTACCCAGAGTCCTTCTGCGTCCGCTAGTC | |
| | TCGGAAATAGTGTTAAGATTACGTGCACTTTGAGTAGCCAACATTCCACTTAT | |
| | ACCATCGGCTGGTATCAGCAACACCCAGATAAAGCCCCGAAATATGTGATGTA | |
| | TGTCAATTCCGACGGTTCTCATTCAAAAGGCGACGGGATCCCCGACAGATTCA | |
| | GCGGTAGTTCATCTGGAGCTCACCGCTATCTGTCAATATCCAATATTCAGCCG | |
| | GAAGATGAAGCTGACTATTTTTGCGGGAGCAGTGATAGTTCCGGTTATGTCTT | |
| | CGGCAGTGGTACACAGCTTACCGTACTCAGAGCAGCCGCTACAACAACTCCTG | |
| | CACCGCGACCTCCAACGCCTGCCCCTACCATTGCGTCACAACCACTTAGCCTT | |
| | AGGCCAGAAGCGTGCCGACCTGCAGCAGGCGGGGCCGTTCATACTAGGGGTCT | |
| | TGACTTCGCGTGCGATATCTACATATGGGCACCTCTGGCGGGAACCTGTGGTG | |
| | TGTTGCTTCTTAGTCTGGTAATTACACTCTACTGTAAACGGGGCAGAAAGAAA | |
| | CTCCTGTATATATTCAAACAACCATTTATGAGACCAGTACAAACTACTCAAGA | |
| | GGAAGATGGCTGTAGCTGCCGATTTCCAGAAGAAGAAGAAGGAGGATGTGAAC | |
| | TGCGCGTGAAGTTTTCACGATCTGCAGATGCCCCGGCATATAAACAAGGTCAG | |
| | AACCAGTTGTACAATGAATTGAACCTTGGTAGAAGGGAGGAGTATGACGTCCT | |
| | CGACAAGAGAAGAGGGCGCGACCCGGAGATGGGCGGTAAGCCAAGGAGAAAAA | |
| | ATCCGCAAGAGGGGCTTTATAATGAACTCCAGAAGGATAAGATGGCTGAGGCT | |
| | TACAGTGAGATTGGTATGAAAGGAGAAAGAAGACGCGGAAAGGGACATGATGG | |
| | GTTGTACCAAGGGCTCAGCACTGCTACCAAGGACACTTATGACGCGCTCCATA | |
| | TGCAAGCCCTTCCCCCTAGATAATAATAAAATCGCTATCCATCGAAGATGGAT | |
| | GTGTGTTGGTTTTTTGTGTGTGGAGCAACAAATCTGACTTTGCATGTGCAAAC | |
| | GCCTTCAACAACAGCATTATTCCAGAAGACACCTTCTTCCCCAGCCCAGGTAA | |
| | GGGCAGCTTTGGTGCCTTCGCAGGCTGTTTCCTTGCTTCAGGAATGGCCAGGT | |
| | TCTGCCCAGAGCTCTGGTCAATGATGTCTAAAACTCCTCTGATTGGTGGTCTC | |
| | GGCCTTATCCATTGCCACCAAAACCCTCTTTTTACTAAGAAACAGTGAGCCTT | |
| | GTTCTGGCAGTCCAGAGAATGACACGGGAAAAAAGCAGATGAAGAGAAGGTGG | |
| | CAGGAGAGGGCACGTGGCCCAGCCTCAGTCTCTCCAACTGAGTTCCTGCCTGC | |
| | CTGCCTTTGCTCAGACTGTTTGCCCCTTACTGCTCTTCTAGGCCTCATTCTAA | |
| | GCCCCTTCTCCAAGTTGCCTCTCCTTATTTCTCCCTGTCTGCCAAAAAATCTT | |
| | TCCCAGCTCACTAAGTCAGTCTCACGCAGTCACTCATTAACCCACCAATCACT | |
| | GATTGTGCCGGCACATGAATGCACCAGGTGTTGAAGTGGAGGAATTAAAAAGT | |
| | CAGATGAGGGGTGTGCCCAGAGGAAGCACCATTCTAGTTGGGGGAGCCCATCT | |
| | GTCAGCTGGGAAAAGTCCAAATAACTTCAGATTGGAATGTGTTTTAACTCAGG | |
| | GTTGAGAAAACAGCTACCTTCAGGACAAAAGTCAGGGAAGGGCTCTCTGAAGA | |
| | AATGCTACTTGAAGATACCAGCCCTACCAAGGGCAGGGAGAGGACCCTATAGA | |
| | GGCCTGGGACAGGAGCTCAATGAGAAAGG | |

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985_{»}; Transcription and Translation (B. D. Hames & S. J. Higgins, eds. (1984_{»}; Animal Cell Culture (R. I. Freshney, ed. (1986_{»}; Immobilized Cells and Enzymes (IRL Press, (1986_{»}; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. Generation of Allogeneic T Cells Expressing an Anti-CD83 Chimeric Antigen Receptor (CAR)

This example describes the production of exemplary allogeneic human T cells that lack expression of the TRAC gene and β2M gene and express a chimeric antigen receptor (CAR) targeting CD83 (anti-CD83 CAR).

Activated primary human T cells were electroporated with Cas9:sgRNA RNP complexes containing TRAC sgRNA (SEQ ID NO: 3) and β2M sgRNA (B2M1 (SEQ ID NO: 7) or B2M4 (SEQ ID NO: 223)) followed by transduction with a recombinant adeno-associated adenoviral vector, serotype 6 (AAV6) (MOI 50, 000) comprising the nucleotide sequence of SEQ ID NO: 66 (encoding anti-CD83 CAR comprising the amino acid sequence of SEQ ID NO: 52 and the LHA/RHA arms) and are referred to as “CD83 CAR+WT”. The sequences of the sgRNAs and donor template are provided in Tables 1, 5, and 6 above. A non-transduced control was generated by electroporating activated T cells with RNP complexes containing the TRAC sgRNA and B2M sgRNA as described above and are referred to as “NO CAR Control”. RNP electroporation conditions are similar to what is described in Hendel et al., Nat Biotechnol. 2015; 33(9):985-989, PMID: 26121415.

### Cell Viability Assay

The number of viable cells and % viability of “CD83 CAR+WT” and the “NO CAR Control” were assessed at Day 2, 4 and 7 and are enumerated in Table 7 (with the B2M4 guide for β2M disruption).

TABLE 7
Cell Viability

| Culture | CD83 CAR + WT | | NO CAR Control | |
|---|---|---|---|---|
| Day | Viable Cell | % | Viable Cell | % |
| post EP | Count | Viability | Count | Viability |
| 2 | 8.4E+04 | 60 | 1.8E+06 | 83 |
| 4 | 1.6E+05 | 75 | 5.7E+06 | 93 |
| 7 | 1.7E+06 | 90 | 1.7E+07 | 96 |

### Cytotoxicity Assay

Cells were cultured until Day 10 and assessed for CAR dependent cytotoxicity. “CD83 CAR+WT” and “NO CAR Control” cells were co-cultured with eFluor670 labeled CD83+K562 cells at effector to target (E:T) ratios of 0.5, 0.25, 0.125, and 0.0625 for 24 hours. After incubation, wells were washed, media was replaced with 200 μL of buffer containing 3 μM DAPI (Thermo, Catalog S34860) and incubated at RT for 20 min. Samples were run on the Attune NxT (Thermo, BYRV6 optical configuration). Data was analyzed using FlowJo (TreeStar). The number of viable K562 cells was determined by counting the number of eFluor670+, DAPI− cells in 150 μL of each sample. The percent cell lysis of the target cells was then determined using the following formula:

(1−((total number of target cells in a test sample)+(total number of target cells in a control sample))×100

The percent cell lysis at each E:T ratio is summarized in Table 8 (with the B2M4 guide for β2M disruption).

TABLE 8
Percentage of Cell Lysis

| | Percentage of Cell Lysis (%) | |
|---|---|---|
| E:T Ratio | CD83 CAR + WT | NO CAR Control |
| 0.5:1 | 41.0 | 11.9 |
| 0.25:1 | 27.2 | 2.6 |
| 0.125:1 | 15.9 | 0.2 |
| 0.0625:1 | 7.1 | −5.4 |

### T Cell Fractions

Cells were cultured until Day 14 and the frequency of CD4 and CD8 cells was determined by flow cytometry.

Taken together, the results from these studies showed a reduced growth rate, viability, and CD8+ frequency in anti-CD83 CAR T cells suggesting the presence of CAR mediated elimination of T cells, a phenomenon referred to as fratricide.

### Example 2: Design and Screening of CD83 Guides

To solve the fratricide problem observed in Example 1 above and improve the rate of CAR T cell growth, and viability, gRNAs were evaluated for their ability disrupt the CD83 gene, protein expression, and improve CAR T cell function. This example describes efficient editing of the CD83 gene in primary human T cells ex vivo using CRISPR/Cas9 gene editing.

Genomic segments of the CD83 gene containing the protein coding exons 2 and 3 were used as input in gRNA design software. The genomic segments also included flanking splice site acceptor/donor sequences. Desired gRNAs were those that would lead to insertions or deletions in the coding sequence, disrupting the amino acid sequence of CD83 leading to out of frame/loss of function allele(s) (referred to as “CD83 knockout alleles” or “CD83 disrupted alleles”). Six (6) in silico-identified gRNA spacer sequences targeting the CD83 gene were synthesized, and the gRNAs were specifically modified, as indicated in Table 2. While the modified gRNAs in Table 2 (with 2′-O-methyl phosphorothioate modifications) were used in the examples, unmodified gRNAs, or gRNAs with other modifications, can be used.

Activated primary human T cells were electroporated with Cas9:sgRNA RNP complexes containing each CD83 sgRNA described in Table 2 using conditions similar to those described in Example 1. A mock electroporated control (no Cas9, no gRNA) was also included. Four days post transfection, cells were stained with PE conjugated anti-human CD83 antibody, Clone HB15e (BioLegend, Catalog 305308) and evaluated by flowcytometry. Percent knock-out values are summarized in Table 9 (% CD83 knock-out by flow). Two guides (exon 2_1 and exon 2_3) showed >95% CD83 disruption by flow. Edited cells using these guides were selected for evaluation by TIDE.

TABLE 9
Gene Editing Efficiency by Various CD83 Guides

| | % CD83 | % CD83 knock-out |
|---|---|---|
| Culture | knock-out by flow | efficiency by TIDE |
| CD83 exon 2-1 | 95.6 | 47.4% |
| CD83 exon 2-3 | ~100.0 | 94.5% |
| Mock | 0.0 | — |

gDNA was isolated from edited cells exhibiting the highest % CD83 editing, “CD83 exon2-1” and CD83 exon 2-3”, as well as the “Mock” cultures. DNA was isolated using the DNeasy Blood & Tissue Kit (Quiagen, Catalog 369506) followed by an initial PCR using the Platinum Supermix HiFI kit (Invitrogen 12532-016). Both gDNA isolation and PCR was performed according to the manufacturer's protocol. The PCR primers used to amplify the CD83 locus are described in Table 10 below. PCR products were sequences and analyzed for insertion and deletion frequencies using a 50 bp window on either side of the predicted CRISPR/Cas9 cleavage site.

TABLE 10
Primers for CD83 amplicon library preparation (TIDE primers)

| | | Used for |
|---|---|---|
| Primer Name | Primer Sequence | sequencing |
| CD83_Exon2_F | acagctctgcagctcgtggc | Yes |
| | (SEQ ID NO: 67) | |
| CD83_Exon2_R | ggatgaaatggcctaagtcacttctgt | No |
| | (SEQ ID NO: 68) | |
| CD83_Exon3_F | tgcttgcagtggaccgtgatg | Yes |
| | (SEQ ID NO: 69) | |
| CD83_Exon3_R | acgcagaggttcttgttctcttggg | No |
| | (SEQ ID NO: 70) | |

The knock-out efficiencies are summarized in Table 9 above (% CD83 knock-out efficiency by TIDE). Based on the knockout data in TABLE 7, gRNA CD83 exon 2-3 was selected to further evaluate the effects of CD83 KO in CAR T cells

### On-Target and Off-Target Editing of CD83 Guide RNAs

Additional CD83 gRNA target sites are selected for further evaluation of on-target and off-target editing efficiencies (Table 11). The on-target and off-target editing efficiencies of the CD83-targeting gRNAs listed in Table 11 are determined as described herein. Briefly, activated T cells are transfected (electroporated) with a ribonucleoprotein particle (RNP) containing Cas9 nuclease and a synthetic modified sgRNA targeting the CD83 gene (sequences in Table 11 below) or controls (no Cas9, no gRNA).

For genomic on- and off-target assessment, these electroporation methods are used to generate cell populations of edited cells from 2-3 different donor T cells. Cells are gene edited with each of the 16 guides noted in Table 11 and then collected ten (10) days post transfection. Sequences of these additional guides and their target sequences are provided in Table 2 above. The samples with the strongest protein knock-down are analyzed with hybrid capture, a method of enrichment of DNA from pre-specified genomic sites, combined with next-generation sequencing. Briefly, on- and off-target sites with homology to each gRNA target site are identified computationally, single-stranded RNA probes are used to enrich these sites from bulk genomic DNA, these enriched sites are sequenced with next-generation sequencing, and the data are analyzed for insertions and deletions (indels) indicating repair following CRISPR editing.

TABLE 11
Additional CD83 Gene Edit Targets

| Guide name | Target Exon |
|---|---|
| CD83_exon2_G1 (a.k.a., CD83-1) | 2 |
| CD83_exon2_G2 (a.k.a., CD83-2) | 2 |
| CD83_exon2_G3 (a.k.a., CD83-3) | 2 |
| CD83_exon2_G4 (a.k.a., CD83-4) | 2 |
| CD83_exon2_G5 (a.k.a., CD83-5) | 2 |
| CD83_exon2_G6 (a.k.a., CD83-6) | 2 |
| CD83_exon3_G7 (a.k.a., CD83-7) | 3 |
| CD83_exon3_G8 (a.k.a., CD83-8) | 3 |
| CD83_exon2_G9 (a.k.a., CD83-9) | 2 |
| CD83_exon3_G10 (a.k.a., CD83-10) | 3 |
| CD83_exon2_G11 (a.k.a., CD83-11) | 2 |
| CD83_exon3_G12 (a.k.a., CD83-12) | 3 |
| CD83_exon2_G13 (a.k.a., CD83-13) | 2 |
| CD83_exon2_G14 (a.k.a., CD83-14) | 2 |
| CD83_exon2_G15 (a.k.a., CD83-15) | 2 |
| CD83_exon2_G16 (a.k.a., CD83-16) | 2 |
| CD83_exon2_1 | 2 |
| CD83_exon2_3 | 2 |
| CD83-17 | 1 |
| CD83-18 | 3 |
| CD83-19 | 1 |
| CD83-20 | 3 |
| CD83-21 | 2 |
| CD83-22 | 2 |
| CD83-23 | 1 |
| CD83-24 | 1 |
| CD83-25 | 4 |

Table 12 below summarizes cut site locations of exemplary CD83 guides.

TABLE 12
Cute Site Locations of Exemplary CD83 Guides

| | | Cutsite Location ^{A} | |
|---|---|---|---|
| Guide Name | Exon | Amino Acid # | Coordinate |
| CD83-17 | 1 | 2 | chr6: 14117815 (−) |
| CD83-19 | | 11 | chr6: 14117843 (−) |
| CD83-23 | | 12 | chr6: 14117848 (+) |
| CD83-24 | | 15 | chr6: 14117955 (−) |
| CD83-22 | 2 | 17 | chr6: 14117962 (−) |
| CD83-6 | | 30 | chr6: 14118000 (+) |
| CD83-14 | | 35 | chr6: 14118017 (−) |
| CD83-9 | | 40 | chr6: 14118029 (−) |
| CD83-13 | | 41 | chr6: 14118033 (+) |
| CD83-4 | | 43 | chr6: 14118038 (−) |
| CD83-1 | | 46 | chr6: 14118047 (−) |
| CD83-2 | | 47 | chr6: 14118053 (+) |
| CD83-21 | | 48 | chr6: 14118054 (+) |
| CD83-20 | 3 | 100 | chr6: 14131664 (−) |
| CD83-8 | | 101 | chr6: 14131667 (+) |
| CD83-12 | | 102 | chr6: 14131668 (+) |
| CD83-18 | | 105 | chr6: 14131678 (+) |
| CD83-10 | | 112 | chr6: 14131701 (+) |
| CD83-7 | | 113 | chr6: 14131702 (+) |
| CD83-25 | 4 | 148 | chr6: 14133708 (+) |

^{A} Use CD83 canonical transcript (ENST00000379153.4); Uniprot: Q01151

### Example 3. Generation and Characterization of Allogeneic Anti-CD83 CAR T Cells Having a CD83 Gene Disruption

This example describes the production of allogeneic human T cells that lack expression of the CD83 gene, TCR gene and β2M gene and express an anti-CD83 CAR.

### Experiment 1

Activated primary human T cells were electroporated with Cas9:sgRNA RNP complexes containing TRAC sgRNA (SEQ ID NO: 3), 32M sgRNA (SEQ ID NO:223), and/or CD83 sgRNA (CD83 exon 2_3, SEQ ID NO: 24) and then transduced with a recombinant adeno-associated adenoviral vector, serotype 6 (AAV6) (MOI 50,000) comprising the nucleotide sequence of SEQ ID NO: 66 (encoding anti-CD83 CAR comprising the amino acid sequence of SEQ ID NO: 52). The sequences of the sgRNAs and donor template are provided in Tables 1, 2 and 6. The resulting engineered cells expressed an anti-CD83 CAR and were deficient in two or more genes, TRAC−, B2M−, and/or CD83−, the resulting populations are listed below:
TRAC−/B2M−/anti-CD83 CAR+ (CD83 CAR T+WT)
TRAC−/B2M−/CD83−/anti-CD83 CAR+(CD83 CAR T−KO)

A non-transduced control was generated by electroporating activated T cells with RNP complexes as described above or with no gene editing elements (referred to as “Mock”). RNP electroporation conditions are similar to what is described in Hendel et al., Nat Biotechnol. 2015; 33(9):985−989, PMID: 26121415.

Impact of CD83 Disruption on Phenotype and Function of Anti-CD83 CAR T Cells In Vitro

At Day 7, the frequency of CD4 and CD8 cells in the population was determined by flow cytometry and is summarized in Table 13.

TABLE 13
Frequency of CD4+ and CD8+ cells at Day 7

| Culture | % CD4+ | % CD8+ |
|---|---|---|
| CD83 CAR + WT | 81.1 | 6.04 |
| CD83 CAR + 2-3 KO | 69.8 | 22.5 |
| Unedited Control | 75.5 | 15.8 |

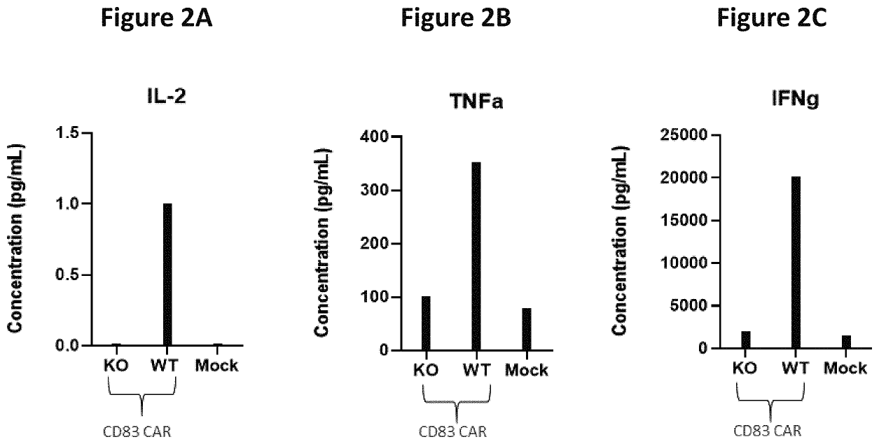

Culture supernatants were collected at Day 7 and levels of IL-2, TNF-α and IFN-7 were quantified using a Luminex based MILLIPLEX assay according to the manufacturer's protocol. The results are provided in FIGS. 2A-2C. These data demonstrate that knocking out CD83 from anti-CD83 CAR T cells reduced levels of pro-inflammatory cytokines (FIGS. 2A-2C), increased CD8+ frequency (Table 12) as compared to CD83 CAR T cells with a functional CD83 gene.

In addition, fold expansion was assessed at Days 3, 5 and 9. As shown in Table 14, disruption of the CD83 gene increased the rate of CAR T cell growth in in CD83 CAR T−KO cells compared to CD83 CAR T+WT cells.

TABLE 14
Fold Expansion of Anti-CD83 CAR T Cells

| | CD83 | CD83 | |
|---|---|---|---|
| Day | CAR | CAR | Unedited |
| post EP | T + WT | T KO | Control |
| 0 | 1.0 | 1.0 | 1.0 |
| 3 | 1.7 | 1.4 | 4.3 |
| 5 | 2.3 | 11.2 | 16.6 |
| 9 | 4.7 | 17.4 | Not |
| | | | determined |

### Cytotoxicity Assay

A cell killing assay was used to assess the ability of the TRAC−/β2M−/CD70−/CD83−/anti-CD83 CAR+ cells to kill a CD83+ cell lines (e.g., K562). TRAC−/β2M−/CD70−/CD83−/anti-CD83 CAR+ cells (KO), TRAC−/β2M−/CD70−/anti-CD83 CAR+ cells (WT) and unedited cells (mock) were co-cultured with eFluor670 labeled CD83+K562 cells at effector to target (E:T) ratios of 2:1, 1:1, 0.5:1, 0.25:1, 0.125:1 or 0.0625:1 and assayed as described above.

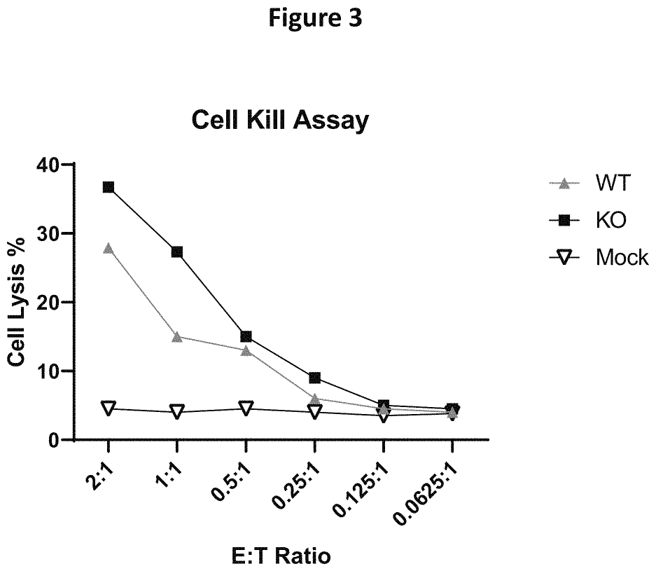

Cells with CD83 disruption exhibited a more potent cell killing of the CD83+ cell line (following 24-hour co-incubation. Knocking out CD83 did not impair CAR mediated killing of CD83+ target cells (FIG. 3).

### Experiment 2

Primary human T cells were thawed and activated with TransAct®. The TransAct® was removed after 3 days and the T cells were electroporated with Cas9:sgRNA RNP complexes containing TRAC sgRNA (SEQ ID NO: 3), 32M sgRNA (SEQ ID NO:223), and/or CD83 sgRNAs listed in Tables 1, 2 and 11 above and then transduced with a recombinant adeno-associated adenoviral vector, serotype 6 (AAV6) (MOI 50,000) comprising the nucleotide sequence of SEQ ID NO: 66 (encoding anti-CD83 CAR comprising the amino acid sequence of SEQ ID NO: 52). The sequences of the sgRNAs and donor template are provided in Tables 1, 2 and 6. The resulting engineered cells expressed an anti-CD83 CAR and were deficient in two or more genes, TRAC−, B2M−, and/or CD83−, the resulting populations are listed in Table 15 below. ‘Y’ indicates the presence of the CAR or knockout of the gene.

TABLE 15
Genetic Editing Events in Various T Cell Populations

| | TRAC | B2M4 | | CD83 |
|---|---|---|---|---|
| Cells | KO | KO | CD83 Guide | CAR |
| NO CAR Control | Y | Y | — | — |
| CD83 CAR + WT | Y | Y | — | Y |
| CD83 CAR + Guide3 | Y | Y | CD83_exon2_G3 | Y |
| CD83 CAR + Guide5 | Y | Y | CD83_exon2_G5 | Y |
| CD83 CAR + Guide6 | Y | Y | CD83_exon2_G6 | Y |
| CD83 CAR + Guide9 | Y | Y | CD83_exon2_G9 | Y |
| CD83 CAR + Guide11 | Y | Y | CD83_exon2_G11 | Y |
| CD83 CAR + Guide15 | Y | Y | CD83_exon2_G15 | Y |
| CD83 CAR + Guide16 | Y | Y | CD83_exon2_G16 | Y |

A non-transduced control was generated by electroporating activated T cells with RNP complexes as described above or with no gene editing elements (referred to as “Mock”). RNP electroporation conditions are similar to what is described in Hendel et al., Nat Biotechnol. 2015; 33(9):985−989, PMID: 26121415.

Impact of CD83 Disruption on Phenotype of Anti-CD83 CAR T Cells In Vitro

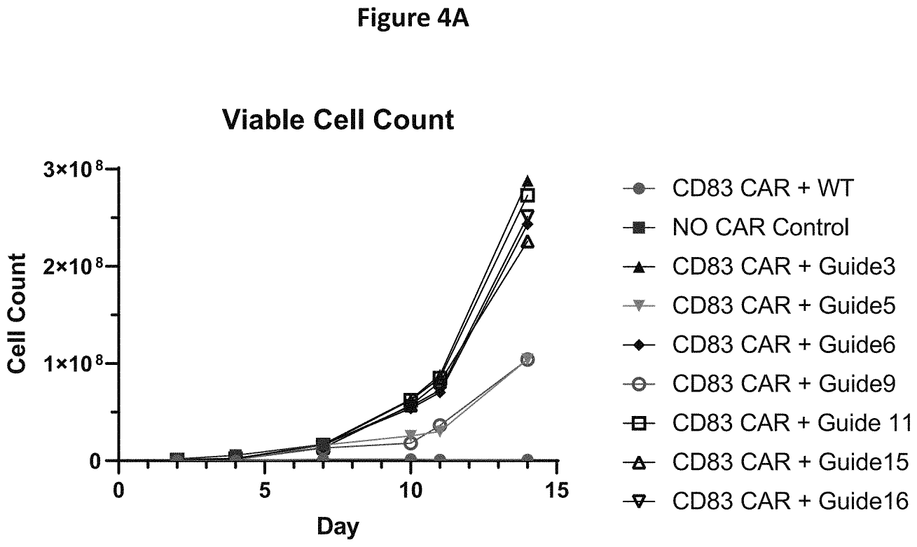

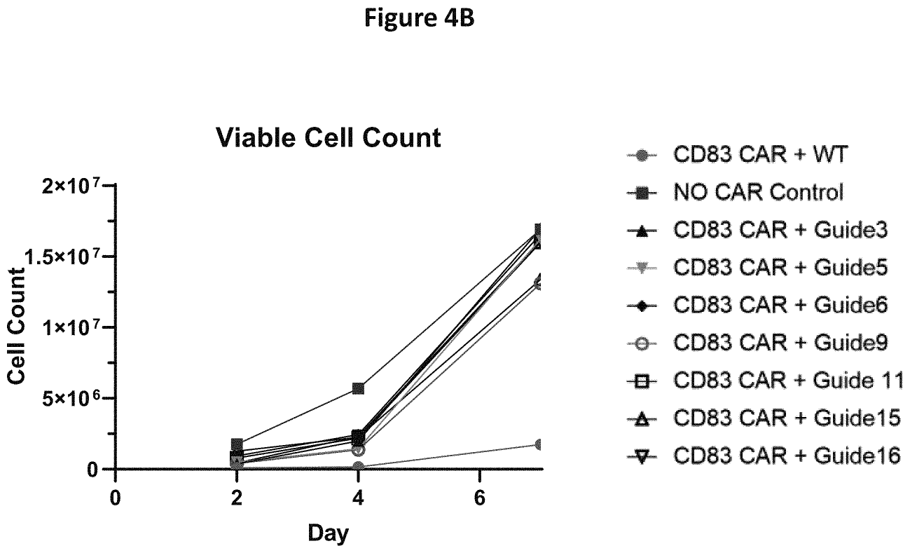

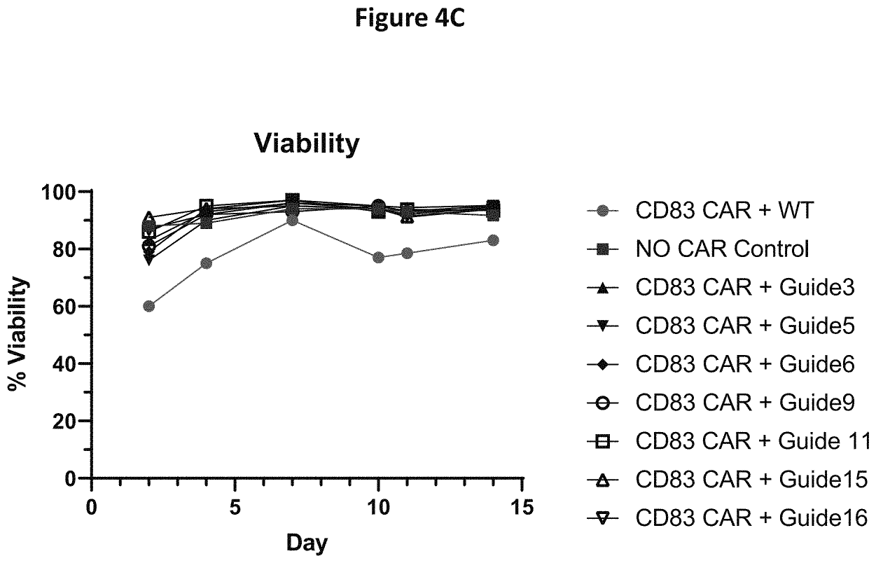

The viable cell count of the different cell cohorts were taken at regular time points. As shown in FIGS. 4A-4C, all the CD83 CAR+ KO cohorts had growth curves similar to the no CAR control, whereas CD83 CAR+WT cells showed lower viability and growth in culture.

Cytotoxicity Assay

A cell killing assay was used to assess the ability of the TRAC−/β2M−/CD70−/CD83-/anti-CD83 CAR+ cells to kill a CD83+ cell lines (e.g., K562). TRAC−/β2M−/CD70−/CD83-/anti-CD83 CAR+ cells (KO), TRAC−/β2M−/CD70−/anti-CD83 CAR+ cells (WT) and unedited cells (No CAR control) were co-cultured with eFluor670 labeled CD83+K562 cells at effector to target (E:T) ratios of 0.5:1, 0.25:1, 0.125:1 or 0.0625:1 and assayed as described above.

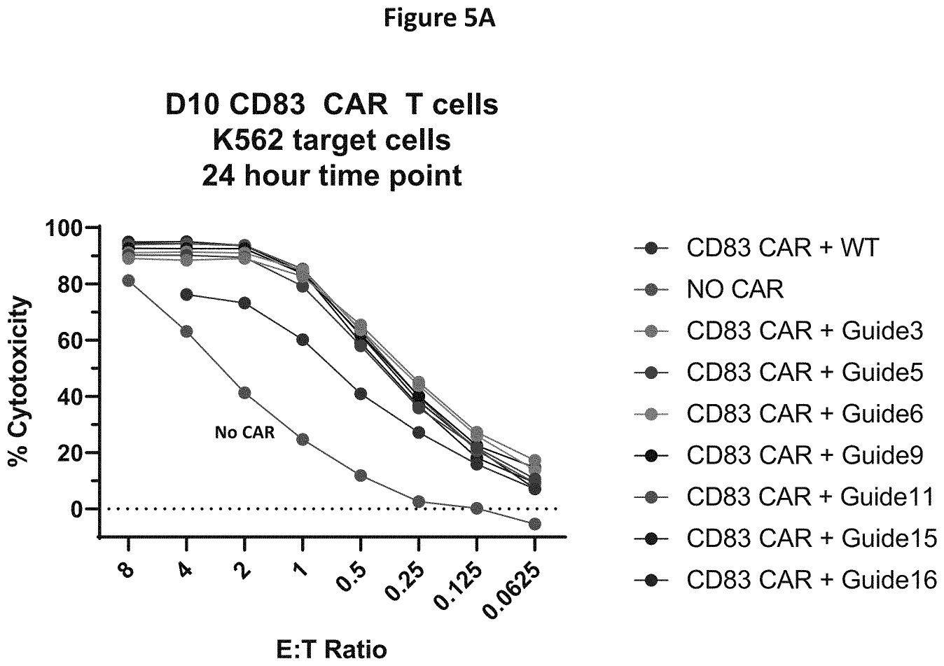

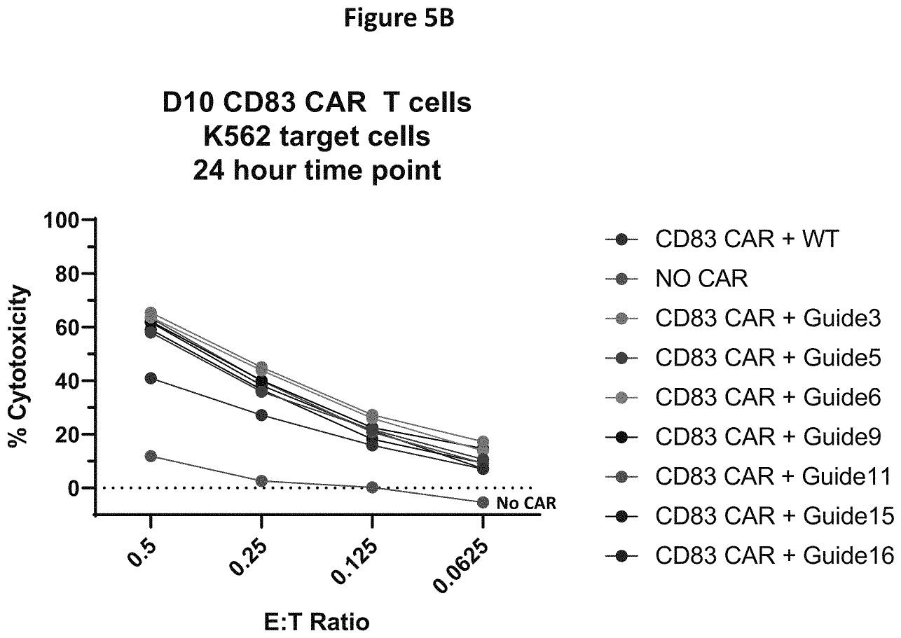

All cell cohorts with CD83 disruption exhibited more potent cell killing of the CD83+ cell line (following 24-hour co-incubation), which showed more potency than CD83 CAR+WT cells and substantially more cytotoxicity than the no CAR control. FIGS. 5A and 5B. Lower TRAC editing efficiency was observed in the no CAR control cells. The TCR⁺ cells may mediate alloreactive killing of the K562 target cells at high E:T ratios.

Taken together, these data suggest that knockout of CD83 in the CAR T cells eliminated CD83-mediated fratricide, specifically of CD8+ T cells, and did not impair CAR T cell function.

### Experiment 3

Primary human T cells were thawed and activated with TransAct®. The TransAct® was removed after 4 days and the T cells electroporated with Cas9:sgRNA RNP complexes containing TRAC sgRNA (SEQ ID NO: 3), 32M sgRNA (B2M1 sgRNA, SEQ ID NO: 7), and/or CD83 sgRNAs listed in Tables 1, 2 and 11 above and then transduced with a recombinant adeno-associated adenoviral vector, serotype 6 (AAV6) (MOI 50,000) comprising the nucleotide sequence of SEQ ID NO: 66 (encoding anti-CD83 CAR comprising the amino acid sequence of SEQ ID NO: 52). The sequences of the sgRNAs and donor template are provided in Tables 1, 2 and 6. The resulting engineered cells expressed an anti-CD83 CAR and were deficient in two or more genes, TRAC−, B2M−, and/or CD83.

Growth and Cell Viability Assays Growth and number of viable cells were assessed and shown in Tables 16−17.

TABLE 16
Total cell count (×10⁷) from 2 donors on Day 10 post-transduction

| Cells | Donor 1 | Donor 2 |
|---|---|---|
| CAR T cell + CD83_exon2_G2 | 6.3 | 4.5 |
| CAR T cell + CD83_exon2_G6 | 6.9 | 3.9 |
| CAR T cell + CD83_exon2_G9 | 6.1 | 4.3 |
| CAR T cell + CD83_17 | 5.5 | 3.8 |
| CAR T cell WT | 3.3 | 1.5 |
| No treatment | 6.2 | 4.9 |

TABLE 17
Percentages of viable cells from 2 donors on Day 3 post-transduction

| Cells | Donor 1 | Donor 2 |
|---|---|---|
| CAR T cell + CD83_exon2_G2 | 92 | 79 |
| CAR T cell + CD83_exon2_G6 | 90 | 77 |
| CAR T cell + CD83_exon2_G9 | 86 | 76 |
| CAR T cell + CD83_17 | 86 | 76 |
| CAR T cell WT | 74 | 69 |
| No treatment | 94 | 94 |

In sum, the results from this experiment showed that disruption of the CD83 gene enhanced viability of CAR T cells.

### Example 4: Impact of CD83 Knockout on Activity of CD83 CAR T Cells in an In Vitro Tumor Rechallenge

This example assesses the effect of disrupting the CD83 gene in CD83 CAR T cells in vitro using a CD83+ tumor cell line.

TRAC−/β2M−/CD70−/CD83−/anti-CD83 CAR+ cells (KO) and TRAC−/β2M−/CD70-/anti-CD83 CAR+ cells (WT) were co-cultured with eFluor670 labeled CD83′ A498 cells at concentrations of 50,000 CAR T cells and 30,000 target cells. The CAR T cells were rechallenged with 60,000 tumor cells on Day 2, 120,000 tumor cells on day 5, and 150,000 tumor cells on day 7. The number of viable target cells and number of viable T cells were counted at regular intervals.

Cells with CD83 disruption showed improved fold-expansion of anti-CD83 CAR T cells while having similar impact on target cell killing in vitro. Table 18 shows data representative of studies done with multiple donors.

TABLE 18
Viable Cell Numbers

| | Target cells | | T cells | |
|---|---|---|---|---|
| Days | KO | WT | KO | WT |
| 2 | 4694 | 479 | 916 | 1641 |
| 5 | 26145 | 20254 | 4270 | 3084 |
| 7 | 55766 | 38603 | 3475 | 1412 |
| 10 | 61725 | 67553 | 2263 | 1145 |

### Example 5: Impact of CD83 Knockout on Activity of CD83 CAR T Cells In Vivo

This example assesses the effect of disrupting the CD83 gene in CD83 CAR T cells in vivo using a xenogeneic graft versus host disease (GvHD) model in mice as well as an established THP-1 xenograft tumor in mice.

### Preventative GvHD Model Study

NSG mice were individually housed in ventilated microisolator cages, maintained under pathogen-free conditions, 5−7 days prior to the start of the study. The mice each received a subcutaneous inoculation of 20×10⁶ PBMCs/mouse to induce GvHD. The mice were further divided into 4 treatment groups and treatment groups 2 to 5 were co-administered a single intravenous dose of T cells according to Table 19. The CAR T cells were made from a donor that was allogeneic to the PBMC donor used to humanize the NSG mice. The CD83_exon2_3 gRNA (see Table 2 above) was used in this study to disrupt the CD83 gene.

TABLE 19
Treatment groups

| | | | T cell | |
|---|---|---|---|---|
| Group | CAR-T | PBMCs | treatment (i.v.) | N |
| 1 | None | 20 × 10⁶ cells/mouse | None | 9 |
| 2 | CD83 CAR | 20 × 10⁶ cells/mouse | 3 × 10⁶ cells/mouse | 9 |
| | WT T cells | | | |
| 3 | CD83 CAR | 20 × 10⁶ cells/mouse | 3 × 10⁶ cells/mouse | 9 |
| | KO T cells | | | |
| 4 | CD83 CAR | 20 × 10⁶ cells/mouse | 1 × 10⁶ cells/mouse | 9 |
| | WT T cells | | | |
| 5 | CD83 CAR | 20 × 10⁶ cells/mouse | 1 × 10⁶ cells/mouse | 9 |
| | KO T cells | | | |

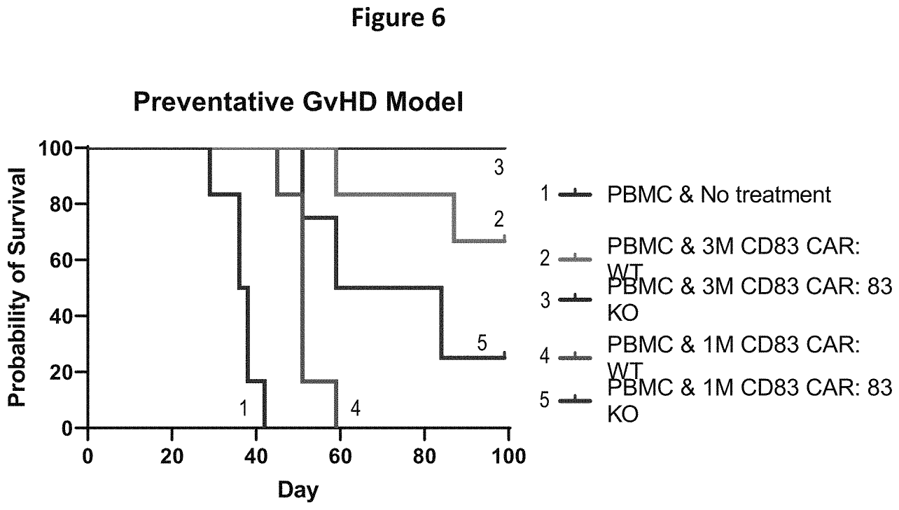

Body weights of the mice were measured at regular intervals and survival of the mice was monitored. Table 20 shows the body weight changes of the mice. FIG. 6 and Table 21 show the survival of the mice groups. It was observed that at 1 million CAR T cell dose level, WT cells increased the median survival of the mice 1.4-fold while CAR T cells with CD83 KO increased median survival 1.9-fold compared to the control group. At the 3 million CAR T cell dose level, CAR T cells with CD83 KO prevented GvHD altogether while WT cells delayed the onset of GvHD. This is consistent with data showing that CD83 CAR T cells subjected to fratricide during expansion exhibit an exhausted phenotype that reduces their potency in vitro and in vivo.

TABLE 20
% Body weight change of mice (compared to Day 0)

| | | PBMC | 3M CAR T | 3M CAR T | 1M CAR T | 1M CAR T |
|---|---|---|---|---|---|---|
| Day | Control | only | WT | KO | WT | KO |
| 9 | 7 | 8 | 5 | 6 | 5 | 4 |
| 15 | 9 | 4 | 7 | 6 | 9 | 7 |
| 17 | 9 | 2 | 8 | 8 | 9 | 7 |
| 22 | 9 | −1 | 8 | 9 | 12 | 10 |
| 24 | 11 | −2 | 10 | 10 | 14 | 12 |
| 29 | 13 | −8 | 10 | 12 | 15 | 12 |
| 31 | 13 | −8 | 11 | 14 | 13 | 12 |
| 36 | 15 | −18 | 15 | 15 | 6 | 13 |
| 38 | 15 | −19 | 15 | 14 | 2 | 10 |
| 42 | 15 | −16 | 16 | 15 | −4 | 6 |
| 45 | 17 | N/A* | 17 | 16 | −7 | 6 |
| 49 | 17 | N/A* | 17 | 16 | −12 | 3 |
| 51 | 17 | N/A* | 17 | 15 | −16 | 0 |
| 56 | 18 | N/A* | 13 | 16 | −11 | 3 |
| 59 | 18 | N/A* | 11 | 17 | −16 | 0 |
| 63 | 18 | N/A* | 21 | 17 | N/A* | 22 |
| 66 | 19 | N/A* | 21 | 18 | N/A* | 22 |
| 70 | 20 | N/A* | 20 | 18 | N/A* | 22 |
| 73 | 22 | N/A* | 23 | 22 | N/A* | 21 |
| 77 | 21 | N/A* | 21 | 20 | N/A* | 19 |
| 80 | 22 | N/A* | 22 | 22 | N/A* | 18 |
| 84 | 24 | N/A* | 22 | 23 | N/A* | 17 |
| 87 | 24 | N/A* | 20 | 24 | N/A* | 14 |
| 91 | 24 | N/A* | 25 | 25 | N/A* | 13 |
| 99 | 24 | N/A* | 23 | 25 | N/A* | 11 |

*No surviving mice

TABLE 21
Survival of the mice

| | Median Survival | GvHD Delay |
|---|---|---|
| Group | (days) | or Prevention? |
| No Treatment | 37 | — |
| 3M CD83 CAR + WT | Not reached | Delay |
| 3M CD83 CAR + 83 KO | Not reached | Prevention |
| 1M CD83 CAR + WT | 51 | Delay |
| 1M CD83 CAR + 83 KO | 71.5 | Delay |

### Established Tumor Xenograft Model Study

The CAR T cells were evaluated in an established THP-1 human acute monocytic leukemia xenograft model in NSG mice. Female NSG mice were subcutaneously implanted on the right flank with 5×10⁶ tumor cells in 50% Matrigel/50% media. Ten days later, when tumor volume was −50 mm³ (range 25−75 mm³), the mice were randomized into 5 groups and injected intravenously with CAR T cells at 10⁷ CAR+ cells per mouse.

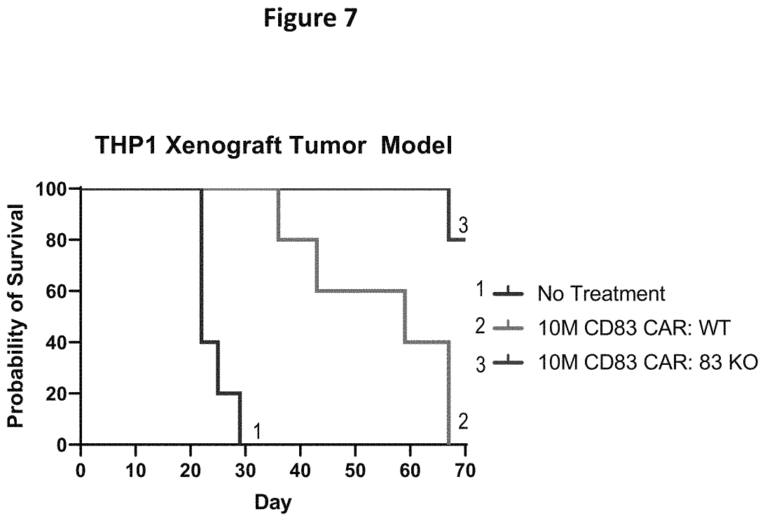

Tumor volumes were evaluated every few days. Tumor volumes are presented in Table 22. Survival of the mice was also evaluated and is presented in Table 23 and FIG. 7. The data demonstrates that knockout of CD83 significantly enhanced the anti-tumor activity of anti-CD83 CAR T cells against THP-1 xenograft tumors.

TABLE 22
Tumor volume (mm³)

| Study | No | 10M CAR | 10M CAR |
|---|---|---|---|
| Day | treatment | T WT | T KO |
| 1 | 53 | 53 | 53 |
| 4 | 135 | 124 | 114 |
| 8 | 297 | 66 | 67 |
| 11 | 574 | 64 | 37 |
| 15 | 937 | 67 | 13 |
| 18 | 1437 | 63 | 9 |
| 22 | 2147 | 93 | 6 |
| 25 | 1965 | 135 | 7 |
| 29 | 2081 | 216 | 18 |
| 32 | N/A* | 346 | 21 |
| 36 | N/A* | 635 | 39 |
| 39 | N/A* | 388 | 38 |
| 43 | N/A* | 702 | 60 |
| 45 | N/A* | 334 | 77 |
| 50 | N/A* | 617 | 125 |
| 53 | N/A* | 865 | 164 |
| 57 | N/A* | 1247 | 229 |
| 61 | N/A* | 1271 | 285 |
| 64 | N/A* | 1647 | 389 |
| 67 | N/A* | 2580 | 611 |
| 71 | N/A* | N/A* | 10 |
| 73 | N/A* | N/A* | 14 |
| 78 | N/A* | N/A* | 17 |
| 81 | N/A* | N/A* | 29 |
| 85 | N/A* | N/A* | 0 |
| 88 | N/A* | N/A* | 0 |
| 92 | N/A* | N/A* | 0 |
| 95 | N/A* | N/A* | 0 |

*No surviving mice

TABLE 23
Survival of the mice

| | Median | # of durable |
|---|---|---|
| | Survival | complete |
| Group | (days) | responses (%) |
| No Treatment | 22 | 0/5 (0%) |
| 10M CD83 CAR + WT | 59 | 0/5 (0%) |
| 10M CD83 CAR + 83 KO | Not reached | 3/5 (60%) |

### Example 6: Bioinformatics Analysis of gRNA Sequences

This example describes the selection of guide sequences targeting the CD83 gene based on on-target editing efficiency analysis and off-target analysis.

The on-target efficiency and average MMEJ rate of 20 guide sequences disclosed herein was reviewed and 10 guides were selected for further off-target analyses via Hybrid Capture and translocation assays. Microhomology-mediated end joining (MMEJ) rate, which calculates the frequency of deletion edits likely resulting from MMEJ repair, was assessed. High indel rate was also an important criterion for selection of a guide RNA. Guides that showed good indel rates and lower MMEJ rates were selected.

TABLE 24
On-Target Efficiency (%) of Guide Sequences

| | Protospacer | Mean | Average |
|---|---|---|---|
| | Sequence | indel | MMEJ |
| Guide | (SEQ ID NO) | rates | rate |
| CD83_exon2_G6 | 82 | 86 | 66 |
| CD83_exon2_G9 | 88 | 83 | 52 |
| CD83_exon2_G14 | 98 | 90 | 25 |
| CD83_exon2_G13 | 96 | 89 | 48 |
| CD83_exon2_G2 | 74 | 92 | 77 |
| CD83_exon2_G1 | 72 | 95 | 28 |
| CD83_exon3_G12 | 94 | 93 | 55 |
| CD83-17 | 169 | 91 | 69 |
| CD83-21 | 173 | 87 | 55 |
| CD83-18 | 171 | 94 | 49 |
| CD83_exon2_G4 | 78 | 89 | 50 |
| CD83_exon3_G8 | 86 | 90 | 46 |
| CD83_exon3_G10 | 90 | 96 | 43 |
| CD83_exon3_G7 | 84 | 93 | 61 |
| CD83-23 | 193 | 92 | 47 |
| CD83-19 | 187 | 90 | 67 |
| CD83-24 | 195 | 95 | 65 |
| CD83-25 | 197 | 53 | 42 |
| CD83-20 | 189 | 30 | 21 |
| CD83-22 | 191 | 20 | 7 |

See Table 2 above for the spacer sequences of the listed gRNAs.

The selected 10 guides were further assessed for off-target editing, as assayed by Hybrid Capture. The results are presented in Tables 25 and 26 below.

TABLE 25
Off-Target Analysis of Guide
Sequences by Hybrid Capture

| | Sites with | |
|---|---|---|
| | >0.2% indel | |
| | rate in least | |
| | 1 donor | True |
| | (edited- | CRISPR |
| Guides | untreated) | off-targets |
| CD83-17 | 0 | 0 |
| CD83_exon2_G2 | 0 | 0 |
| CD83_exon2_G9 | 1 | 1 |
| CD83_exon2_G6 | 1 | 1 |
| CD83_exon2_G1 | 3 | 1 |
| CD83-21 | 1 | 1 |
| CD83-18 | 3 | 2 |
| CD83_exon3_G12 | 1 | 1 |
| CD83_exon2_G13 | 6 | 2 |
| CD83_exon2_G14 | 11 | 8 |

Translocation assessment of selected guides were also done. T cells from 2 donors were thawed and activated. Regnase-1 (Reg-1) and CD70 gRNAs have previously been assessed with a measurable translocation rate and were used in this study as controls. The pairing the CD83 gRNA with either the Regnase-1 gRNA or CD70 gRNA resulted in measurable translocation rates of both and yielded the rank ordering of CD83 guide translocation propensity.

On day 5, cells were electroporated with CD83 guide RNA and a guide RNA targeting Regnase-1 or CD83 guide RNA and a guide targeting CD70. On day 12, the cells were harvested, DNA extracted, and translocation profiling conducted. The results in Table 26 show that CD83−17 and CD83_exon2_G2 (CD83−2) have minimal to no off-target translocation events.

TABLE 26
Translocation Analysis of Guide Sequences

| | CD83 gRNA + Regnase-1 | | | CD83 gRNA + CD70 | | | |
|---|---|---|---|---|---|---|---|
| | gRNA | | | gRNA | | | |
| | CD83 | Reg-1 | CD83/Reg-l | CD83 | CD70 | CD83/CD70 | |
| gRNA | % indels | % indels | translocation | % indels | % indels | translocation | Notes |
| CD83-17 | 100 | 99 | 1.1 | 99 | 99 | 2.5 | Moderate- |
| | | | | | | | low |
| CD83_exon2_G2 | 98 | 98 | 1.4 | 97 | 99 | 1.9 | Low |
| CD83_exon2_G9 | 97 | 98 | 1.2 | 97 | 99 | 1.8 | Low |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A population of genetically engineered immune cells, comprising a disrupted CD83 gene.

2. The population of genetically engineered immune cells of claim 1, wherein the immune cells are further engineered to express a chimeric antigen receptor (CAR).

3. The population of genetically engineered immune cells of claim 1 or claim 2, which comprise T cells.

4. The population of genetically engineered immune cells of claim 1, wherein the disrupted CD83 gene is genetically edited in one or more of exon 1, exon 2, exon 3, an exon 4; optionally wherein the disrupted CD83 gene is genetically edited in exon 2.

5. The population of genetically engineered immune cells of claim 1, wherein the disrupted CD83 gene is genetically edited by a CRISPR/Cas-mediated gene editing system.

6. The population of genetically engineered immune cells of claim 5, wherein the CRISPR/Cas-mediated gene editing system comprises a guide RNA (gRNA) targeting a site in the CD83 gene.

7. The population of genetically engineered immune cells of claim 6, wherein the site in the CD83 gene comprises a nucleotide sequence selected from the group consisting of SEQ ID NOs: 22, 28, 72, 74, 76, 78, 80, 82, 84, 86, 88, 90, 92, 94, 96, 98, 161, 167, 169, 171, 173, 187, 189, 191, 193, 195, and 197; optionally wherein the site in the CD83 gene comprises SEQ ID NO: 28, 74, 88, or 169.

8. The population of genetically engineered immune cells of claim 6, wherein the gRNA comprises a spacer having a nucleotide sequence selected from the group consisting of SEQ ID NOs: 19, 25, 101, 105, 109, 113, 117, 121, 125, 129, 133, 137, 141, 145, 149, 153, 158, 164, 176, 180, 184, 200, 204, 208, 212, 216, and 220; optionally the spacer has the nucleotide sequence of SEQ ID NO: 25, 105, 133, or 176.

9. The population of genetically engineered immune cells of claim 6, wherein the gRNA further comprises a scaffold sequence, which optionally comprises the nucleotide sequence of SEQ ID NO: 155 or SEQ ID NO: 234.

10. The population of genetically engineered immune cells of claim 6, wherein the gRNA comprises a nucleotide sequence selected from the group consisting of SEQ ID NOs: 17, 18, 23, 24, 99, 100, 103, 104, 107, 108, 111, 112, 115, 116, 119, 120, 123, 124, 127, 128, 131, 132, 135, 136, 139, 140, 143, 144, 147, 148, 151, 152, 156, 157, 162, 163, 174, 175, 178, 179, 182, 183, 198, 199, 202, 203, 206, 207, 210, 211, 214, 215, 218, and 219; optionally wherein the gRNA comprises the nucleotide sequence of SEQ ID NO: 23, 24, 103, 104, 131, 132, 174, or 175.

11. The population of genetically engineered immune cells of claim 3, wherein the immune cells are T cells further comprising a disrupted T cell receptor alpha chain constant region (TRAC) gene, a disrupted beta-2-microglobulin (β2M) gene, or a combination thereof.

12. The population of genetically engineered immune cells of claim 11, wherein:
the disrupted TRAC gene is genetically edited by a CRISPR/Cas-mediated gene editing system, which comprises a gRNA comprising the nucleotide sequence of SEQ ID NO: 4, and/or
the disrupted β2M gene is genetically edited by a CRISPR/Cas-mediated gene editing system, which comprises a gRNA comprising the nucleotide sequence of SEQ ID NO: 8 or SEQ ID NO: 224.

13. The population of genetically engineered immune cells of claim 2, wherein the T cells comprise a nucleic acid encoding the CAR.

14. The population of genetically engineered immune cells of claim 13, wherein the nucleic acid encoding the CAR is inserted in a genomic site of interest in the T cells.

15. The population of genetically engineered immune cells of claim 14, wherein the genomic site of interest is the TRAC gene.

16. The population of genetically engineered immune cells of claim 15, wherein the disrupted TRAC gene comprises the nucleic acid encoding the CAR.

17-19. (canceled)

20. The population of genetically engineered immune cells of claim 2, wherein the CAR comprises an extracellular antigen binding domain specific to an antigen, a co-stimulatory signaling domain of 4−1BB or CD28, and a cytoplasmic signaling domain of CD3ζ.

21. The population of genetically engineered immune cells of claim 20, wherein the antigen is CD83.

22. The population of genetically engineered immune cells of claim 21, wherein the extracellular antigen binding domain is a single chain variable fragment (scFv) that binds CD83 (anti-CD83 scFv), wherein the anti-CD83 scFv comprises a heavy chain variable region (V_{H}) and a light chain variable region (V_{L}), and optionally wherein the V_{H} and V_{L} are connected via a peptide linker.

23. The population of genetically engineered immune cells of claim 22, wherein the V_{H} comprises heavy chain complementary determining region (CDR) 1, CDR2, and CDR3 set forth as SEQ ID NOs: 43, 44, and 45, respectively; and/or wherein the V_{L} comprises light chain complementary determining region (CDR) 1, CDR2, and CDR3 set forth as SEQ ID NOs: 46, 47, and 48, respectively.

24. The population of genetically engineered immune cells of claim 23, wherein the V_{H} comprises the amino acid sequence of SEQ ID NO: 49, and/or wherein the V_{L} comprises the amino acid sequence of SEQ ID NO: 50.

25. The population of genetically engineered immune cells of claim 24, wherein the anti-CD83 scFv comprises the amino acid sequence of SEQ ID NO: 51.

26. The population of genetically engineered immune cells of claim 25, wherein the CAR that binds CD83 comprises the amino acid sequence of SEQ ID NO: 52 or SEQ ID NO: 235.

27. The population of genetically engineered immune cells of claim 1, wherein the genetically engineered immune cells are derived from one or more human donors.

28. A population of genetically engineered immune cells, comprising genetically engineered T cells that comprise:
(a) a disrupted TRAC gene, which is genetically edited at a TRAC target site of SEQ ID NO: 11;
(b) a disrupted b2M gene, which is genetically edited at a β2M target site of SEQ ID NO: 13;
(c) a disrupted CD83 gene, which is genetically edited at a CD83 target site of SEQ ID NO: 74; and
(d) a nucleic acid encoding an anti-CD83 CAR, which comprises an anti-CD83 scFv that comprises a V_{H} fragment set forth as SEQ ID NO: 49 and a V_{L} fragment set forth as SEQ ID NO: 50;
wherein the nucleic acid encoding the anti-CD83 CAR is inserted into the disrupted TRAC gene.

29. The population of genetically engineered immune cells of claim 28, wherein:
(a) the disrupted TRAC gene is produced by CRISPR/Cas9-mediated gene editing comprising a gRNA targeting SEQ ID NO: 11;
(b) the disrupted b2M gene is produced by CRISPR/Cas9-mediated gene editing comprising a gRNA targeting SEQ ID NO: 13; and/or
(c) the disrupted CD83 gene is produced by CRISPR/Cas9-mediated gene editing comprising a gRNA targeting SEQ ID NO: 74.

30. The population of genetically engineered immune cells of claim 28, wherein the anti-CD83 scFv comprises the amino acid sequence of SEQ ID NO: 51.

31. The population of genetically engineered immune cells of claim 28, wherein the anti-CD83 CAR comprises the amino acid sequence of SEQ ID NO: 235 or SEQ ID NO: 52.

32. The population of genetically engineered immune cells of claim 28, wherein the disrupted TRAC gene comprises the nucleotide sequence of SEQ ID NO: 66.

33. A method for preparing the population of genetically engineered immune cells of claim 1, the method comprising:
(a) providing a plurality of immune cells, which optionally comprise T cells or precursor cells thereof;
(b) genetically editing a CD83 gene of the immune cells; and
(c) producing the population of genetically engineered immune cells having a disrupted CD83 gene.

34-52. (canceled)

53. A method for eliminating undesired cells in a subject, the method comprising administering to a subject in need thereof a first population of genetically engineered immune cells comprising a disrupted CD83 gene and expressing a first chimeric antigen receptor (CAR) targeting the undesired cells.

54-59. (canceled)

60. A guide RNA (gRNA) targeting a CD83 gene, comprising a nucleotide sequence specific to a fragment in exon 1, exon 2, exon 3, or exon 4 of the CD83 gene, optionally wherein the gRNA comprises a nucleotide sequence specific to exon 2 of the CD83 gene.

61-65. (canceled)


SEQUENCE LISTING (235 sequences)

SEQ ID NO: 1 - protein, 1368 aa, synthetic construct [sequence omitted]

SEQ ID NO: 2 - RNA, 100 nt, synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 3 - RNA, 100 nt, synthetic construct; identical to SEQ ID NO: 2
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 4 - RNA, 20 nt, synthetic construct
  source 1..20: mol_type = other RNA
agagcaacagtgctgtggcc

SEQ ID NO: 5 - RNA, 20 nt, synthetic construct
  source 1..20: mol_type = other RNA
  misc_feature 1..4: note = modified with 2'-O-methyl phosphorothioate
agagcaacagtgctgtggcc

SEQ ID NO: 6 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 7 - RNA, 100 nt, synthetic construct; identical to SEQ ID NO: 6
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 8 - RNA, 20 nt, synthetic construct
  source 1..20: mol_type = other RNA
gctactctctctttctggcc

SEQ ID NO: 9 - RNA, 20 nt, synthetic construct
  source 1..20: mol_type = other RNA
  misc_feature 1..4: note = modified with 2'-O-methyl phosphorothioate
gctactctctctttctggcc

SEQ ID NO: 10 - DNA, 23 bp, synthetic construct
  source 1..23: mol_type = other DNA
agagcaacagtgctgtggcctgg

SEQ ID NO: 11 - DNA, 20 bp, synthetic construct
  source 1..20: mol_type = other DNA
agagcaacagtgctgtggcc

SEQ ID NO: 12 - DNA, 23 bp, synthetic construct
  source 1..23: mol_type = other DNA
gctactctctctttctggcctgg

SEQ ID NO: 13 - DNA, 20 bp, synthetic construct
  source 1..20: mol_type = other DNA
gctactctctctttctggcc

SEQ ID NO: 14 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 15 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 16 - RNA, 114 nt, synthetic construct
  source 1..114: mol_type = other RNA
  misc_feature 18..30: note = a, g, c, t, or absent
  misc_feature 108..114: note = t or absent
nnnnnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 17 - RNA, 103 nt, synthetic construct
  source 1..103: mol_type = other RNA
tcccgcgacgccggaggtgaagggttttagagctagaaatagcaagttaaaataaggctagtccgttatcaacttgaaaaagtggcaccgagtcggtgctttt

SEQ ID NO: 18 - RNA, 103 nt, synthetic construct; identical to SEQ ID NO: 17
  source 1..103: mol_type = other RNA
  misc_feature 1..4: note = modified with 2'-O-methyl phosphorothioate
  misc_feature 100..103: note = modified with 2'-O-methyl phosphorothioate

SEQ ID NO: 19 - RNA, 23 nt, synthetic construct
  source 1..23: mol_type = other RNA
tcccgcgacgccggaggtgaagg

SEQ ID NO: 20 - RNA, 23 nt, synthetic construct
  source 1..23: mol_type = other RNA
  misc_feature 1..4: note = modified with 2'-O-methyl phosphorothioate
tcccgcgacgccggaggtgaagg

SEQ ID NO: 21 - DNA, 26 bp, synthetic construct
  source 1..26: mol_type = other DNA
tcccgcgacgccggaggtgaaggtgg

SEQ ID NO: 22 - DNA, 23 bp, synthetic construct
  source 1..23: mol_type = other DNA
tcccgcgacgccggaggtgaagg

SEQ ID NO: 23 - RNA, 103 nt, synthetic construct
  source 1..103: mol_type = other RNA
gtagggaacctgcggatcccagggttttagagctagaaatagcaagttaaaataaggctagtccgttatcaacttgaaaaagtggcaccgagtcggtgctttt

SEQ ID NO: 24 - RNA, 103 nt, synthetic construct; identical to SEQ ID NO: 23
  source 1..103: mol_type = other RNA
  misc_feature 1..4: note = modified with 2'-O-methyl phosphorothioate
  misc_feature 100..103: note = modified with 2'-O-methyl phosphorothioate

SEQ ID NO: 25 - RNA, 23 nt, synthetic construct
  source 1..23: mol_type = other RNA
gtagggaacctgcggatcccagg

SEQ ID NO: 26 - RNA, 23 nt, synthetic construct
  source 1..23: mol_type = other RNA
  misc_feature 1..4: note = modified with 2'-O-methyl phosphorothioate
gtagggaacctgcggatcccagg

SEQ ID NO: 27 - DNA, 26 bp, synthetic construct
  source 1..26: mol_type = other DNA
gtagggaacctgcggatcccaggggg

SEQ ID NO: 28 - DNA, 23 bp, synthetic construct
  source 1..23: mol_type = other DNA
gtagggaacctgcggatcccagg

SEQ ID NO: 29 - DNA, 19 bp, synthetic construct
  source 1..19: mol_type = other DNA
aagagcaacaaatctgact

SEQ ID NO: 30 - DNA, 39 bp, synthetic construct
  source 1..39: mol_type = other DNA
aagagcaacagtgctgtgcctggagcaacaaatctgact

SEQ ID NO: 31 - DNA, 33 bp, synthetic construct
  source 1..33: mol_type = other DNA
aagagcaacagtgctggagcaacaaatctgact

SEQ ID NO: 32 - DNA, 34 bp, synthetic construct
  source 1..34: mol_type = other DNA
aagagcaacagtgcctggagcaacaaatctgact

SEQ ID NO: 33 - DNA, 19 bp, synthetic construct
  source 1..19: mol_type = other DNA
aagagcaacagtgctgact

SEQ ID NO: 34 - DNA, 41 bp, synthetic construct
  source 1..41: mol_type = other DNA
aagagcaacagtgctgtgggcctggagcaacaaatctgact

SEQ ID NO: 35 - DNA, 38 bp, synthetic construct
  source 1..38: mol_type = other DNA
aagagcaacagtgctggcctggagcaacaaatctgact

SEQ ID NO: 36 - DNA, 41 bp, synthetic construct
  source 1..41: mol_type = other DNA
aagagcaacagtgctgtgtgcctggagcaacaaatctgact

SEQ ID NO: 37 - DNA, 79 bp, synthetic construct
  source 1..79: mol_type = other DNA
cgtggccttagctgtgctcgcgctactctctctttctgcctggaggctatccagcgtgagtctctcctaccctcccgct

SEQ ID NO: 38 - DNA, 78 bp, synthetic construct
  source 1..78: mol_type = other DNA
cgtggccttagctgtgctcgcgctactctctctttcgcctggaggctatccagcgtgagtctctcctaccctcccgct

SEQ ID NO: 39 - DNA, 75 bp, synthetic construct
  source 1..75: mol_type = other DNA
cgtggccttagctgtgctcgcgctactctctctttctggaggctatccagcgtgagtctctcctaccctcccgct

SEQ ID NO: 40 - DNA, 84 bp, synthetic construct
  source 1..84: mol_type = other DNA
cgtggccttagctgtgctcgcgctactctctctttctggatagcctggaggctatccagcgtgagtctctcctaccctcccgct

SEQ ID NO: 41 - DNA, 55 bp, synthetic construct
  source 1..55: mol_type = other DNA
cgtggccttagctgtgctcgcgctatccagcgtgagtctctcctaccctcccgct

SEQ ID NO: 42 - DNA, 82 bp, synthetic construct
  source 1..82: mol_type = other DNA
cgtggccttagctgtgctcgcgctactctctctttctgtggcctggaggctatccagcgtgagtctctcctaccctcccgct

SEQ ID NO: 43 - protein, 7 aa, synthetic construct
TGGYWWT

SEQ ID NO: 44 - protein, 16 aa, synthetic construct
YIFSSGNTNYNPSIKS

SEQ ID NO: 45 - protein, 9 aa, synthetic construct
AYGKLGFDY

SEQ ID NO: 46 - protein, 12 aa, synthetic construct
TLSSQHSTYTIG

SEQ ID NO: 47 - protein, 11 aa, synthetic construct
VNSDGSHSKGD

SEQ ID NO: 48 - protein, 9 aa, synthetic construct
GSSDSSGYV

SEQ ID NO: 49 - protein, 119 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVS

SEQ ID NO: 50 - protein, 111 aa, synthetic construct
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 51 - protein, 246 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 52 - protein, 494 aa, synthetic construct
MALPVTALLLPLALLLHAARPQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 21 aa, synthetic construct
MALPVTALLLPLALLLHAARP

SEQ ID NO: 54 - protein, 22 aa, synthetic construct
MLLLVTSLLLCELPHPAFLLIP

SEQ ID NO: 55 - protein, 42 aa, synthetic construct
KRGRKKLLYIFKQPFMRPVQTTQEEDGCSCRFPEEEEGGCEL

SEQ ID NO: 56 - protein, 40 aa, synthetic construct
SKRSRLLHSDYMNMTPRRPGPTRKHYQPYAPPRDFAAYRS

SEQ ID NO: 57 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 59 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 60 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 61 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 62 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 63 - DNA, 800 bp, synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 64 - DNA, 804 bp, synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 65 - DNA, 1178 bp, synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 66 - DNA, 4322 bp, synthetic construct
  source 1..4322: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggttcagctgaaagaatctggccccggcctggttaaaccgagtcagagtttgagcttgacttgttctgtgactggattctcaataacaaccggtggatactggtggacctggatacgacaatttccgggccaaaagctcgagtggatgggctatatctttagtagcggcaacactaactataatccttcaatcaagagcaggataagtatcacccgagacaccagtaagaaccaattctttttgcagcttaacagcgttacgactgaaggtgacaccgcaaggtattactgcgcgagggcctacggaaaacttggttttgattattggggtcaagggacactggtaacggtttctagtgggggaggtggtagtggcggtggaggttccgggggtggaggatcccaacccgtccttacccagagtccttctgcgtccgctagtctcggaaatagtgttaagattacgtgcactttgagtagccaacattccacttataccatcggctggtatcagcaacacccagataaagccccgaaatatgtgatgtatgtcaattccgacggttctcattcaaaaggcgacgggatccccgacagattcagcggtagttcatctggagctcaccgctatctgtcaatatccaatattcagccggaagatgaagctgactatttttgcgggagcagtgatagttccggttatgtcttcggcagtggtacacagcttaccgtactcagagcagccgctacaacaactcctgcaccgcgacctccaacgcctgcccctaccattgcgtcacaaccacttagccttaggccagaagcgtgccgacctgcagcaggcggggccgttcatactaggggtcttgacttcgcgtgcgatatctacatatgggcacctctggcgggaacctgtggtgtgttgcttcttagtctggtaattacactctactgtaaacggggcagaaagaaactcctgtatatattcaaacaaccatttatgagaccagtacaaactactcaagaggaagatggctgtagctgccgatttccagaagaagaagaaggaggatgtgaactgcgcgtgaagttttcacgatctgcagatgccccggcatataaacaaggtcagaaccagttgtacaatgaattgaaccttggtagaagggaggagtatgacgtcctcgacaagagaagagggcgcgacccggagatgggcggtaagccaaggagaaaaaatccgcaagaggggctttataatgaactccagaaggataagatggctgaggcttacagtgagattggtatgaaaggagaaagaagacgcggaaagggacatgatgggttgtaccaagggctcagcactgctaccaaggacacttatgacgcgctccatatgcaagcccttccccctagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 67 - DNA, 20 bp, synthetic construct
  source 1..20: mol_type = other DNA
acagctctgcagctcgtggc

SEQ ID NO: 68 - DNA, 27 bp, synthetic construct
  source 1..27: mol_type = other DNA
ggatgaaatggcctaagtcacttctgt

SEQ ID NO: 69 - DNA, 21 bp, synthetic construct
  source 1..21: mol_type = other DNA
tgcttgcagtggaccgtgatg

SEQ ID NO: 70 - DNA, 24 bp, synthetic construct
  source 1..24: mol_type = other DNA
acgcagaggttcttgttctcttgg

SEQ ID NO: 71 - DNA, 23 bp, synthetic construct
  source 1..23: mol_type = other DNA
ttgacccaggagaccgtgtaggg

SEQ ID NO: 72 - DNA, 20 bp, synthetic construct
  source 1..20: mol_type = other DNA
ttgacccaggagaccgtgta

SEQ ID NO: 73 - DNA, 23 bp, synthetic construct
  source 1..23: mol_type = other DNA
aggttccctacacggtctcctgg

SEQ ID NO: 74 - DNA, 20 bp, synthetic construct
  source 1..20: mol_type = other DNA
aggttccctacacggtctcc

SEQ ID NO: 75 - DNA, 23 bp, synthetic construct
  source 1..23: mol_type = other DNA
ggatccgcaggttccctacacgg

SEQ ID NO: 76 - DNA, 20 bp, synthetic construct
  source 1..20: mol_type = other DNA
ggatccgcaggttccctaca

SEQ ID NO: 77 - DNA, 23 bp, synthetic construct
  source 1..23: mol_type = other DNA
gagaccgtgtagggaacctgcgg

SEQ ID NO: 78 - DNA, 20 bp, synthetic construct
  source 1..20: mol_type = other DNA
gagaccgtgtagggaacctg

SEQ ID NO: 79 - DNA, 23 bp, synthetic construct
  source 1..23: mol_type = other DNA
cttgacccaggagaccgtgtagg

SEQ ID NO: 80 - DNA, 20 bp, synthetic construct
  source 1..20: mol_type = other DNA
cttgacccaggagaccgtgt

SEQ ID NO: 81 - DNA, 23 bp, synthetic construct
  source 1..23: mol_type = other DNA
ggtggcttgctccgaagatgtgg

SEQ ID NO: 82 - DNA, 20 bp, synthetic construct
  source 1..20: mol_type = other DNA
ggtggcttgctccgaagatg

SEQ ID NO: 83 - DNA, 23 bp, synthetic construct
  source 1..23: mol_type = other DNA
gcactctgcaggacccggatggg

SEQ ID NO: 84 - DNA, 20 bp, synthetic construct
  source 1..20: mol_type = other DNA
gcactctgcaggacccggat

SEQ ID NO: 85 - DNA, 23 bp, synthetic construct
  source 1..23: mol_type = other DNA
aaacactaccagctgcaactcgg

SEQ ID NO: 86 - DNA, 20 bp, synthetic construct
  source 1..20: mol_type = other DNA
aaacactaccagctgcaact

SEQ ID NO: 87 - DNA, 23 bp, synthetic construct
  source 1..23: mol_type = other DNA
tagggaacctgcggatcccaggg

SEQ ID NO: 88 - DNA, 20 bp, synthetic construct
  source 1..20: mol_type = other DNA
tagggaacctgcggatccca

SEQ ID NO: 89 - DNA, 23 bp, synthetic construct
  source 1..23: mol_type = other DNA
tgcactctgcaggacccggatgg

SEQ ID NO: 90 - DNA, 20 bp, synthetic construct
  source 1..20: mol_type = other DNA
tgcactctgcaggacccgga

SEQ ID NO: 91 - DNA, 23 bp, synthetic construct
  source 1..23: mol_type = other DNA
acttgccctgcaccgccccctgg

SEQ ID NO: 92 - DNA, 20 bp, synthetic construct
  source 1..20: mol_type = other DNA
acttgccctgcaccgccccc

SEQ ID NO: 93 - DNA, 23 bp, synthetic construct
  source 1..23: mol_type = other DNA
aacactaccagctgcaactcggg

SEQ ID NO: 94 - DNA, 20 bp, synthetic construct
  source 1..20: mol_type = other DNA
aacactaccagctgcaactc

SEQ ID NO: 95 - DNA, 23 bp, synthetic construct
  source 1..23: mol_type = other DNA
caccgccccctgggatccgcagg

SEQ ID NO: 96 - DNA, 20 bp, synthetic construct
  source 1..20: mol_type = other DNA
caccgccccctgggatccgc

SEQ ID NO: 97 - DNA, 23 bp, synthetic construct
  source 1..23: mol_type = other DNA
ggatcccagggggcggtgcaggg

SEQ ID NO: 98 - DNA, 20 bp, synthetic construct
  source 1..20: mol_type = other DNA
ggatcccagggggcggtgca

SEQ ID NO: 99 - RNA, 97 nt, synthetic construct
  source 1..97: mol_type = other RNA
ttgacccaggagaccgtgtagttttagagctagaaatagcaagttaaaataaggctagtccgttatcaacttgaaaaagtggcaccgagtcggtgct

SEQ ID NO: 100 - RNA, 97 nt, synthetic construct; identical to SEQ ID NO: 99
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 101 - RNA, 20 nt, synthetic construct
  source 1..20: mol_type = other RNA
ttgacccaggagaccgtgta

SEQ ID NO: 102 - RNA, 20 nt, synthetic construct
  source 1..20: mol_type = other RNA
  misc_feature 1..4: note = modified with 2'-O-methyl phosphorothioate
ttgacccaggagaccgtgta

SEQ ID NO: 103 - RNA, 97 nt, synthetic construct
  source 1..97: mol_type = other RNA
aggttccctacacggtctccgttttagagctagaaatagcaagttaaaataaggctagtccgttatcaacttgaaaaagtggcaccgagtcggtgct

SEQ ID NO: 104 - RNA, 97 nt, synthetic construct; identical to SEQ ID NO: 103
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 105 - RNA, 20 nt, synthetic construct
  source 1..20: mol_type = other RNA
aggttccctacacggtctcc

SEQ ID NO: 106 - RNA, 20 nt, synthetic construct
  source 1..20: mol_type = other RNA
  misc_feature 1..4: note = modified with 2'-O-methyl phosphorothioate
aggttccctacacggtctcc

SEQ ID NO: 107 - RNA, 97 nt, synthetic construct
  source 1..97: mol_type = other RNA
ggatccgcaggttccctacagttttagagctagaaatagcaagttaaaataaggctagtccgttatcaacttgaaaaagtggcaccgagtcggtgct

SEQ ID NO: 108 - RNA, 97 nt, synthetic construct; identical to SEQ ID NO: 107
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 109 - RNA, 20 nt, synthetic construct
  source 1..20: mol_type = other RNA
ggatccgcaggttccctaca

SEQ ID NO: 110 - RNA, 20 nt, synthetic construct
  source 1..20: mol_type = other RNA
  misc_feature 1..4: note = modified with 2'-O-methyl phosphorothioate
ggatccgcaggttccctaca

SEQ ID NO: 111 - RNA, 97 nt, synthetic construct
  source 1..97: mol_type = other RNA
gagaccgtgtagggaacctggttttagagctagaaatagcaagttaaaataaggctagtccgttatcaacttgaaaaagtggcaccgagtcggtgct

SEQ ID NO: 112 - RNA, 97 nt, synthetic construct; identical to SEQ ID NO: 111
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 113 - RNA, 20 nt, synthetic construct
  source 1..20: mol_type = other RNA
gagaccgtgtagggaacctg

SEQ ID NO: 114 - RNA, 20 nt, synthetic construct
  source 1..20: mol_type = other RNA
  misc_feature 1..4: note = modified with 2'-O-methyl phosphorothioate
gagaccgtgtagggaacctg

SEQ ID NO: 115 - RNA, 97 nt, synthetic construct
  source 1..97: mol_type = other RNA
cttgacccaggagaccgtgtgttttagagctagaaatagcaagttaaaataaggctagtccgttatcaacttgaaaaagtggcaccgagtcggtgct

SEQ ID NO: 116 - RNA, 97 nt, synthetic construct; identical to SEQ ID NO: 115
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 117 - RNA, 20 nt, synthetic construct
  source 1..20: mol_type = other RNA
cttgacccaggagaccgtgt

SEQ ID NO: 118 - RNA, 20 nt, synthetic construct
  source 1..20: mol_type = other RNA
  misc_feature 1..4: note = modified with 2'-O-methyl phosphorothioate
cttgacccaggagaccgtgt

SEQ ID NO: 119 - RNA, 97 nt, synthetic construct
  source 1..97: mol_type = other RNA
ggtggcttgctccgaagatggttttagagctagaaatagcaagttaaaataaggctagtccgttatcaacttgaaaaagtggcaccgagtcggtgct

SEQ ID NO: 120 - RNA, 97 nt, synthetic construct; identical to SEQ ID NO: 119
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 121 - RNA, 20 nt, synthetic construct
  source 1..20: mol_type = other RNA
ggtggcttgctccgaagatg

SEQ ID NO: 122 - RNA, 20 nt, synthetic construct
  source 1..20: mol_type = other RNA
  misc_feature 1..4: note = modified with 2'-O-methyl phosphorothioate
ggtggcttgctccgaagatg

SEQ ID NO: 123 - RNA, 97 nt, synthetic construct
  source 1..97: mol_type = other RNA
gcactctgcaggacccggatgttttagagctagaaatagcaagttaaaataaggctagtccgttatcaacttgaaaaagtggcaccgagtcggtgct

SEQ ID NO: 124 - RNA, 97 nt, synthetic construct; identical to SEQ ID NO: 123
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 125 - RNA, 20 nt, synthetic construct
  source 1..20: mol_type = other RNA
gcactctgcaggacccggat

SEQ ID NO: 126 - RNA, 20 nt, synthetic construct
  source 1..20: mol_type = other RNA
  misc_feature 1..4: note = modified with 2'-O-methyl phosphorothioate
gcactctgcaggacccggat

SEQ ID NO: 127 - RNA, 97 nt, synthetic construct
  source 1..97: mol_type = other RNA
aaacactaccagctgcaactgttttagagctagaaatagcaagttaaaataaggctagtccgttatcaacttgaaaaagtggcaccgagtcggtgct

SEQ ID NO: 128 - RNA, 97 nt, synthetic construct; identical to SEQ ID NO: 127
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 129 - RNA, 20 nt, synthetic construct
  source 1..20: mol_type = other RNA
aaacactaccagctgcaact

SEQ ID NO: 130 - RNA, 20 nt, synthetic construct
  source 1..20: mol_type = other RNA
  misc_feature 1..4: note = modified with 2'-O-methyl phosphorothioate
aaacactaccagctgcaact

SEQ ID NO: 131 - RNA, 97 nt, synthetic construct
  source 1..97: mol_type = other RNA
tagggaacctgcggatcccagttttagagctagaaatagcaagttaaaataaggctagtccgttatcaacttgaaaaagtggcaccgagtcggtgct

SEQ ID NO: 132 - RNA, 97 nt, synthetic construct; identical to SEQ ID NO: 131
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 133 - RNA, 20 nt, synthetic construct
  source 1..20: mol_type = other RNA
tagggaacctgcggatccca

SEQ ID NO: 134 - RNA, 20 nt, synthetic construct
  source 1..20: mol_type = other RNA
  misc_feature 1..4: note = modified with 2'-O-methyl phosphorothioate
tagggaacctgcggatccca

SEQ ID NO: 135 - RNA, 97 nt, synthetic construct
  source 1..97: mol_type = other RNA
tgcactctgcaggacccggagttttagagctagaaatagcaagttaaaataaggctagtccgttatcaacttgaaaaagtggcaccgagtcggtgct

SEQ ID NO: 136 - RNA, 97 nt, synthetic construct; identical to SEQ ID NO: 135
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 137 - RNA, 20 nt, synthetic construct
  source 1..20: mol_type = other RNA
tgcactctgcaggacccgga

SEQ ID NO: 138 - RNA, 20 nt, synthetic construct
  source 1..20: mol_type = other RNA
  misc_feature 1..4: note = modified with 2'-O-methyl phosphorothioate
tgcactctgcaggacccgga

SEQ ID NO: 139 - RNA, 97 nt, synthetic construct
  source 1..97: mol_type = other RNA
acttgccctgcaccgcccccgttttagagctagaaatagcaagttaaaataaggctagtccgttatcaacttgaaaaagtggcaccgagtcggtgct

SEQ ID NO: 140 - RNA, 97 nt, synthetic construct; identical to SEQ ID NO: 139
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 141 - RNA, 20 nt, synthetic construct
  source 1..20: mol_type = other RNA
acttgccctgcaccgccccc

SEQ ID NO: 142 - RNA, 20 nt, synthetic construct
  source 1..20: mol_type = other RNA
  misc_feature 1..4: note = modified with 2'-O-methyl phosphorothioate
acttgccctgcaccgccccc

SEQ ID NO: 143 - RNA, 97 nt, synthetic construct
  source 1..97: mol_type = other RNA
aacactaccagctgcaactcgttttagagctagaaatagcaagttaaaataaggctagtccgttatcaacttgaaaaagtggcaccgagtcggtgct

SEQ ID NO: 144 - RNA, 97 nt, synthetic construct; identical to SEQ ID NO: 143
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 145 - RNA, 20 nt, synthetic construct
  source 1..20: mol_type = other RNA
aacactaccagctgcaactc

SEQ ID NO: 146 - RNA, 20 nt, synthetic construct
  source 1..20: mol_type = other RNA
  misc_feature 1..4: note = modified with 2'-O-methyl phosphorothioate
aacactaccagctgcaactc

SEQ ID NO: 147 - RNA, 97 nt, synthetic construct
  source 1..97: mol_type = other RNA
caccgccccctgggatccgcgttttagagctagaaatagcaagttaaaataaggctagtccgttatcaacttgaaaaagtggcaccgagtcggtgct

SEQ ID NO: 148 - RNA, 97 nt, synthetic construct; identical to SEQ ID NO: 147
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 149 - RNA, 20 nt, synthetic construct
  source 1..20: mol_type = other RNA
caccgccccctgggatccgc

SEQ ID NO: 150 - RNA, 20 nt, synthetic construct
  source 1..20: mol_type = other RNA
  misc_feature 1..4: note = modified with 2'-O-methyl phosphorothioate
caccgccccctgggatccgc

SEQ ID NO: 151 - RNA, 97 nt, synthetic construct
  source 1..97: mol_type = other RNA
ggatcccagggggcggtgcagttttagagctagaaatagcaagttaaaataaggctagtccgttatcaacttgaaaaagtggcaccgagtcggtgct

SEQ ID NO: 152 - RNA, 97 nt, synthetic construct; identical to SEQ ID NO: 151
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 153 - RNA, 20 nt, synthetic construct
  source 1..20: mol_type = other RNA
ggatcccagggggcggtgca

SEQ ID NO: 154 - RNA, 20 nt, synthetic construct
  source 1..20: mol_type = other RNA
  misc_feature 1..4: note = modified with 2'-O-methyl phosphorothioate
ggatcccagggggcggtgca

SEQ ID NO: 155 - RNA, 77 nt, synthetic construct
  source 1..77: mol_type = other RNA
  misc_feature 1..4: note = modified with 2'-O-methyl phosphorothioate
  misc_feature 74..77: note = modified with 2'-O-methyl phosphorothioate
gttttagagctagaaatagcaagttaaaataaggctagtccgttatcaacttgaaaaagtggcaccgagtcggtgct

SEQ ID NO: 156 - RNA, 97 nt, synthetic construct
  source 1..97: mol_type = other RNA
tcccgcgacgccggaggtgagttttagagctagaaatagcaagttaaaataaggctagtccgttatcaacttgaaaaagtggcaccgagtcggtgct

SEQ ID NO: 157 - RNA, 97 nt, synthetic construct; identical to SEQ ID NO: 156
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 158 - RNA, 20 nt, synthetic construct
  source 1..20: mol_type = other RNA
tcccgcgacgccggaggtga

SEQ ID NO: 159 - RNA, 20 nt, synthetic construct
  source 1..20: mol_type = other RNA
  misc_feature 1..4: note = modified with 2'-O-methyl phosphorothioate
tcccgcgacgccggaggtga

SEQ ID NO: 160 - DNA, 23 bp, synthetic construct
  source 1..23: mol_type = other DNA
tcccgcgacgccggaggtgaagg

SEQ ID NO: 161 - DNA, 20 bp, synthetic construct
  source 1..20: mol_type = other DNA
tcccgcgacgccggaggtga

SEQ ID NO: 162 - RNA, 97 nt, synthetic construct
  source 1..97: mol_type = other RNA
gtagggaacctgcggatcccgttttagagctagaaatagcaagttaaaataaggctagtccgttatcaacttgaaaaagtggcaccgagtcggtgct

SEQ ID NO: 163 - RNA, 97 nt, synthetic construct; identical to SEQ ID NO: 162
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 164 - RNA, 20 nt, synthetic construct
  source 1..20: mol_type = other RNA
gtagggaacctgcggatccc

SEQ ID NO: 165 - RNA, 20 nt, synthetic construct
  source 1..20: mol_type = other RNA
  misc_feature 1..4: note = modified with 2'-O-methyl phosphorothioate
gtagggaacctgcggatccc

SEQ ID NO: 166 - DNA, 23 bp, synthetic construct
  source 1..23: mol_type = other DNA
gtagggaacctgcggatcccagg

SEQ ID NO: 167 - DNA, 20 bp, synthetic construct
  source 1..20: mol_type = other DNA
gtagggaacctgcggatccc

SEQ ID NO: 168 - DNA, 23 bp, synthetic construct
  source 1..23: mol_type = other DNA
aagctggaggccgcgcgacatgg

SEQ ID NO: 169 - DNA, 20 bp, synthetic construct
  source 1..20: mol_type = other DNA
aagctggaggccgcgcgaca

SEQ ID NO: 170 - DNA, 23 bp, synthetic construct
  source 1..23: mol_type = other DNA
gctgcaactcggggacatacagg

SEQ ID NO: 171 - DNA, 20 bp, synthetic construct
  source 1..20: mol_type = other DNA
gctgcaactcggggacatac

SEQ ID NO: 172 - DNA, 23 bp, synthetic construct
  source 1..23: mol_type = other DNA
ggttccctacacggtctcctggg

SEQ ID NO: 173 - DNA, 20 bp, synthetic construct
  source 1..20: mol_type = other DNA
ggttccctacacggtctcct

SEQ ID NO: 174 - RNA, 97 nt, synthetic construct
  source 1..97: mol_type = other RNA
aagctggaggccgcgcgacagttttagagctagaaatagcaagttaaaataaggctagtccgttatcaacttgaaaaagtggcaccgagtcggtgct

SEQ ID NO: 175 - RNA, 97 nt, synthetic construct; identical to SEQ ID NO: 174
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 176 - RNA, 20 nt, synthetic construct
  source 1..20: mol_type = other RNA
aagctggaggccgcgcgaca

SEQ ID NO: 177 - RNA, 20 nt, synthetic construct
  source 1..20: mol_type = other RNA
  misc_feature 1..4: note = modified with 2'-O-methyl phosphorothioate
aagctggaggccgcgcgaca

SEQ ID NO: 178 - RNA, 97 nt, synthetic construct
  source 1..97: mol_type = other RNA
gctgcaactcggggacatacgttttagagctagaaatagcaagttaaaataaggctagtccgttatcaacttgaaaaagtggcaccgagtcggtgct

SEQ ID NO: 179 - RNA, 97 nt, synthetic construct; identical to SEQ ID NO: 178
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 180 - RNA, 20 nt, synthetic construct
  source 1..20: mol_type = other RNA
gctgcaactcggggacatac

SEQ ID NO: 181 - RNA, 20 nt, synthetic construct
  source 1..20: mol_type = other RNA
  misc_feature 1..4: note = modified with 2'-O-methyl phosphorothioate
gctgcaactcggggacatac

SEQ ID NO: 182 - RNA, 97 nt, synthetic construct
  source 1..97: mol_type = other RNA
ggttccctacacggtctcctgttttagagctagaaatagcaagttaaaataaggctagtccgttatcaacttgaaaaagtggcaccgagtcggtgct

SEQ ID NO: 183 - RNA, 97 nt, synthetic construct; identical to SEQ ID NO: 182
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 184 - RNA, 20 nt, synthetic construct
  source 1..20: mol_type = other RNA
ggttccctacacggtctcct

SEQ ID NO: 185 - RNA, 20 nt, synthetic construct
  source 1..20: mol_type = other RNA
  misc_feature 1..4: note = modified with 2'-O-methyl phosphorothioate
ggttccctacacggtctcct

SEQ ID NO: 186 - DNA, 23 bp, synthetic construct
  source 1..23: mol_type = other DNA
cgcgagccctaccgcagctcagg

SEQ ID NO: 187 - DNA, 20 bp, synthetic construct
  source 1..20: mol_type = other DNA
cgcgagccctaccgcagctc

SEQ ID NO: 188 - DNA, 23 bp, synthetic construct
  source 1..23: mol_type = other DNA
gtatgtccccgagttgcagctgg

SEQ ID NO: 189 - DNA, 20 bp, synthetic construct
  source 1..20: mol_type = other DNA
gtatgtccccgagttgcagc

SEQ ID NO: 190 - DNA, 23 bp, synthetic construct
  source 1..23: mol_type = other DNA
cctccggcgtcgcgggagccacg

SEQ ID NO: 191 - DNA, 20 bp, synthetic construct
  source 1..20: mol_type = other DNA
cctccggcgtcgcgggagcc

SEQ ID NO: 192 - DNA, 23 bp, synthetic construct
  source 1..23: mol_type = other DNA
tctgctcctgagctgcggtaggg

SEQ ID NO: 193 - DNA, 20 bp, synthetic construct
  source 1..20: mol_type = other DNA
tctgctcctgagctgcggta

SEQ ID NO: 194 - DNA, 23 bp, synthetic construct
  source 1..23: mol_type = other DNA
cgtcgcgggagccaggctgtagg

SEQ ID NO: 195 - DNA, 20 bp, synthetic construct
  source 1..20: mol_type = other DNA
cgtcgcgggagccaggctgt

SEQ ID NO: 196 - DNA, 23 bp, synthetic construct
  source 1..23: mol_type = other DNA
agcggagattgtcctgctgctgg

SEQ ID NO: 197 - DNA, 20 bp, synthetic construct
  source 1..20: mol_type = other DNA
agcggagattgtcctgctgc

SEQ ID NO: 198 - RNA, 97 nt, synthetic construct
  source 1..97: mol_type = other RNA
cgcgagccctaccgcagctcgttttagagctagaaatagcaagttaaaataaggctagtccgttatcaacttgaaaaagtggcaccgagtcggtgct

SEQ ID NO: 199 - RNA, 97 nt, synthetic construct; identical to SEQ ID NO: 198
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 200 - RNA, 20 nt, synthetic construct
  source 1..20: mol_type = other RNA
cgcgagccctaccgcagctc

SEQ ID NO: 201 - RNA, 20 nt, synthetic construct
  source 1..20: mol_type = other RNA
  misc_feature 1..4: note = modified with 2'-O-methyl phosphorothioate
cgcgagccctaccgcagctc

SEQ ID NO: 202 - RNA, 97 nt, synthetic construct
  source 1..97: mol_type = other RNA
gtatgtccccgagttgcagcgttttagagctagaaatagcaagttaaaataaggctagtccgttatcaacttgaaaaagtggcaccgagtcggtgct

SEQ ID NO: 203 - RNA, 97 nt, synthetic construct; identical to SEQ ID NO: 202
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 204 - RNA, 20 nt, synthetic construct
  source 1..20: mol_type = other RNA
gtatgtccccgagttgcagc

SEQ ID NO: 205 - RNA, 20 nt, synthetic construct
  source 1..20: mol_type = other RNA
  misc_feature 1..4: note = modified with 2'-O-methyl phosphorothioate
gtatgtccccgagttgcagc

SEQ ID NO: 206 - RNA, 97 nt, synthetic construct
  source 1..97: mol_type = other RNA
cctccggcgtcgcgggagccgttttagagctagaaatagcaagttaaaataaggctagtccgttatcaacttgaaaaagtggcaccgagtcggtgct

SEQ ID NO: 207 - RNA, 97 nt, synthetic construct; identical to SEQ ID NO: 206
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 208 - RNA, 20 nt, synthetic construct
  source 1..20: mol_type = other RNA
cctccggcgtcgcgggagcc

SEQ ID NO: 209 - RNA, 20 nt, synthetic construct
  source 1..20: mol_type = other RNA
  misc_feature 1..4: note = modified with 2'-O-methyl phosphorothioate
cctccggcgtcgcgggagcc

SEQ ID NO: 210 - RNA, 97 nt, synthetic construct
  source 1..97: mol_type = other RNA
tctgctcctgagctgcggtagttttagagctagaaatagcaagttaaaataaggctagtccgttatcaacttgaaaaagtggcaccgagtcggtgct

SEQ ID NO: 211 - RNA, 97 nt, synthetic construct; identical to SEQ ID NO: 210
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 212 - RNA, 20 nt, synthetic construct
  source 1..20: mol_type = other RNA
tctgctcctgagctgcggta

SEQ ID NO: 213 - RNA, 20 nt, synthetic construct
  source 1..20: mol_type = other RNA
  misc_feature 1..4: note = modified with 2'-O-methyl phosphorothioate
tctgctcctgagctgcggta

SEQ ID NO: 214 - RNA, 97 nt, synthetic construct
  source 1..97: mol_type = other RNA
cgtcgcgggagccaggctgtgttttagagctagaaatagcaagttaaaataaggctagtccgttatcaacttgaaaaagtggcaccgagtcggtgct

SEQ ID NO: 215 - RNA, 97 nt, synthetic construct; identical to SEQ ID NO: 214
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 216 - RNA, 20 nt, synthetic construct
  source 1..20: mol_type = other RNA
cgtcgcgggagccaggctgt

SEQ ID NO: 217 - RNA, 20 nt, synthetic construct
  source 1..20: mol_type = other RNA
  misc_feature 1..4: note = modified with 2'-O-methyl phosphorothioate
cgtcgcgggagccaggctgt

SEQ ID NO: 218 - RNA, 97 nt, synthetic construct
  source 1..97: mol_type = other RNA
agcggagattgtcctgctgcgttttagagctagaaatagcaagttaaaataaggctagtccgttatcaacttgaaaaagtggcaccgagtcggtgct

SEQ ID NO: 219 - RNA, 97 nt, synthetic construct; identical to SEQ ID NO: 218
  source 1..97: mol_type = other RNA
  misc_feature 1..4: note = modified with 2'-O-methyl phosphorothioate
  misc_feature 94..97: note = modified with 2'-O-methyl phosphorothioate

SEQ ID NO: 220 - RNA, 20 nt, synthetic construct
  source 1..20: mol_type = other RNA
agcggagattgtcctgctgc

SEQ ID NO: 221 - RNA, 20 nt, synthetic construct
  source 1..20: mol_type = other RNA
  misc_feature 1..4: note = modified with 2'-O-methyl phosphorothioate
agcggagattgtcctgctgc

SEQ ID NO: 222 - RNA, 100 nt, synthetic construct
  source 1..100: mol_type = other RNA
cagtaagtcaacttcaatgtgttttagagctagaaatagcaagttaaaataaggctagtccgttatcaacttgaaaaagtggcaccgagtcggtgctttt

SEQ ID NO: 223 - RNA, 100 nt, synthetic construct; identical to SEQ ID NO: 222
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 224 - RNA, 20 nt, synthetic construct
  source 1..20: mol_type = other RNA
cagtaagtcaacttcaatgt

SEQ ID NO: 225 - RNA, 20 nt, synthetic construct
  source 1..20: mol_type = other RNA
  misc_feature 1..4: note = modified with 2'-O-methyl phosphorothioate
cagtaagtcaacttcaatgt

SEQ ID NO: 226 - RNA, 100 nt, synthetic construct
  source 1..100: mol_type = other RNA
gctttggtcccattggtcgcgttttagagctagaaatagcaagttaaaataaggctagtccgttatcaacttgaaaaagtggcaccgagtcggtgctttt

SEQ ID NO: 227 - RNA, 100 nt, synthetic construct; identical to SEQ ID NO: 226
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 228 - RNA, 20 nt, synthetic construct
  source 1..20: mol_type = other RNA
gctttggtcccattggtcgc

SEQ ID NO: 229 - RNA, 20 nt, synthetic construct
  source 1..20: mol_type = other RNA
  misc_feature 1..4: note = modified with 2'-O-methyl phosphorothioate
gctttggtcccattggtcgc

SEQ ID NO: 230 - DNA, 23 bp, synthetic construct
  source 1..23: mol_type = other DNA
cagtaagtcaacttcaatgttgg

SEQ ID NO: 231 - DNA, 20 bp, synthetic construct
  source 1..20: mol_type = other DNA
cagtaagtcaacttcaatgt

SEQ ID NO: 232 - DNA, 23 bp, synthetic construct
  source 1..23: mol_type = other DNA
gctttggtcccattggtcgcggg

SEQ ID NO: 233 - DNA, 20 bp, synthetic construct
  source 1..20: mol_type = other DNA
gctttggtcccattggtcgc

SEQ ID NO: 234 - RNA, 80 nt, synthetic construct
  source 1..80: mol_type = other RNA
  misc_feature 77..80: note = modified with 2'-O-methyl phosphorothioate
gttttagagctagaaatagcaagttaaaataaggctagtccgttatcaacttgaaaaagtggcaccgagtcggtgctttt

SEQ ID NO: 235 - protein, 473 aa, synthetic construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR